SEQUENCE LISTING (10 sequences)

SEQ ID NO: 1 - DNA, 4623 bp, Homo sapiens
  CDS (1)..(4623)
atgctgagccatggagccgggttggccttgtggatcacactgagcctgctgcagactggactggcggagccagagagatgtaacttcaccctggcggagtccaaggcctccagccattctgtgtctatccagtggagaattttgggctcaccctgtaactttagcctcatctatagcagtgacaccctgggggccgcgttgtgccctacctttcggatagacaacaccacatacggatgtaaccttcaagatttacaagcaggaaccatctataacttcaagattatttctctggatgaagagagaactgtggtcttgcaaacagatcctttacctcctgctaggtttggagtcagtaaagagaagacgacttcaaccggcttgcatgtttggtggactccttcttccggaaaagtcacctcatatgaggtgcaattatttgatgaaaataaccaaaagatacagggggttcaaattcaagaaagtacttcatggaatgaatacacttttttcaatctcactgctggtagtaaatacaatattgccatcacagctgtttctggaggaaaacgttctttttcagtttataccaatggatcaacagtgccatctccagtgaaagatattggtatttccacaaaagccaattctctcctgatttcctggtcccatggttctgggaatgtggaacgataccggctgatgctaatggataaagggatcctagttcatggcggtgttgtggacaaacatgctacttcctatgcttttcacgggctgacccctggctacctctacaacctcactgttatgactgaggctgcagggctgcaaaactacaggtggaaactagtcaggacagcccccatggaagtctcaaatctgaaggtgacaaatgatggcagtttgacctctctaaaagtcaaatggcaaagacctcctggaaatgtggattcttacaatatcaccctgtctcacaaagggaccatcaaggaatccagagtattagcaccttggattactgaaactcactttaaagagttagtccccggtcgactttatcaagttactgtcagctgtgtctctggtgaactgtctgctcagaagatggcagtgggcagaacattccccctggctgtcctccagcttcgtgtcaaacatgccaatgaaacctcactgagtatcatgtggcagacccctgtagcagaatgggagaaatacatcatttccctagctgacagagacctcttactgatccacaagtcactctccaaagatgccaaagaattcacttttactgacctggtgcctggacgaaaatacatggctacagtcaccagtattagtggagacttaaaaaattcctcttcagtaaaaggaagaacagtgcctgcccaagtgactgacttgcatgtggccaaccaaggaatgaccagtagtctgtttactaactggacccaggcacaaggagacgtagaattttaccaagtcttactgatccatgaaaatgtggtcattaaaaatgaaagcatctccagtgagaccagcagatacagcttccactctctcaagtccggcagcctgtactccgtggtggtaacaacagtgagtggagggatctcttcccgacaagtggttgtggagggaagaacagtcccttccagtgtgagtggagtaacggtgaacaattccggtcgtaatgactacctcagcgtttcctggctgctggcgcccggagatgtggataactatgaggtaacattgtctcatgacggcaaggtggttcagtcccttgtcattgccaagtctgtcagagaatgttccttcagctccctcaccccaggccgcctctacaccgtgaccataactacaaggagtggcaagtatgaaaatcactccttcagccaagagcggacagtgcctgacaaagtccagggagtcagtgttagcaactcagccaggagtgactatttaagggtatcctgggtgcatgccactggagactttgatcactatgaagtcaccattaaaaacaaaaacaacttcattcaaactaaaagcattcccaagtcagaaaacgaatgtgtatttgttcagctagtccctggacggttgtacagtgtcactgttactacaaaaagtggacaatatgaagccaatgaacaagggaatgggagaacaattccagagcctgttaaggatctaacattgcgcaacaggagcactgaggacttgcatgtgacttggtcaggagctaatggggatgtcgaccaatatgagatccagctgctcttcaatgacatgaaagtatttcctccttttcaccttgtaaataccgcaaccgagtatcgatttacttccctaacaccaggccgccaatacaaaattcttgtcttgacgattagcggggatgtacagcagtcagccttcattgagggcttcacagttcctagtgctgtcaaaaatattcacatttctcccaatggagcaacagatagcctgacggtgaactggactcctggtgggggagacgttgattcctacacggtgtcggcattcaggcacagtcaaaaggttgactctcagactattcccaagcacgtctttgagcacacgttccacagactggaggccggggagcagtaccagatcatgattgcctcagtcagcgggtccctgaagaatcagataaatgtggttgggcggacagttccagcatctgtccaaggagtaattgcagacaatgcatacagcagttattccttaatagtaagttggcaaaaagctgctggtgtggcagaaagatatgatatcctgcttctaactgaaaatggaatccttctgcgcaacacatcagagccagccaccactaagcaacacaaatttgaagatctaacaccaggcaagaaatacaagatacagatcctaactgtcagtggaggcctctttagcaaggaagcccagactgaaggccgaacagtcccagcagctgtcaccgacctgaggatcacagagaactccaccaggcacctgtccttccgctggaccgcctcagagggggagctcagctggtacaacatctttttgtacaacccagatgggaatctccaggagagagctcaagttgacccactagtccagagcttctctttccagaacttgctacaaggcagaatgtacaagatggtgattgtaactcacagtggggagctgtctaatgagtctttcatatttggtagaacagtcccagcctctgtgagtcatctcagggggtccaatcggaacacgacagacagcctttggttcaactggagtccagcctctggggactttgacttttatgagctgattctctataatcccaatggcacaaagaaggaaaactggaaagacaaggacctgacggagtggcggtttcaaggccttgttcctggaaggaagtacgtgctgtgggtggtaactcacagtggagatctcagcaataaagtcacagcggagagcagaacagctccaagtcctcccagtcttatgtcatttgctgacattgcaaacacatccttggccatcacgtggaaagggcccccagactggacagactacaacgactttgagctgcagtggttgcccagagatgcacttactgtcttcaacccctacaacaacagaaaatcagaaggacgcattgtgtatggtcttcgtccagggagatcctatcaattcaacgtcaagactgtcagtggtgattcctggaaaacttacagcaaaccaatttttggatctgtgaggacaaagcctgacaagatacaaaacctgcattgccggcctcagaactccacggccattgcctgttcttggatccctcctgattctgactttgatggttatagtattgaatgccggaaaatggacacccaagaagttgagttttccagaaagctggagaaagaaaaatctctgctcaacatcatgatgctagtgccccataagaggtacctggtgtccatcaaagtgcagtcggccggcatgaccagcgaggtggttgaagacagcactatcacaatgatagaccgcccccctcctccacccccacacattcgtgtgaatgaaaaggatgtgctaattagcaagtcttccatcaactttactgtcaactgcagctggttcagcgacaccaatggagctgtgaaatacttcacagtggtggtgagagaggctgatggcagtgatgagctgaagccagaacagcagcaccctctcccttcctacctggagtacaggcacaatgcctccattcgggtgtatcagactaattattttgccagcaaatgtgccgaaaatcctaacagcaactccaagagttttaacattaagcttggagcagagatggagagcctaggtggaaaatgcgatcccactcagcaaaaattctgtgatggaccactgaagccacacactgcctacagaatcagcattcgagcttttacacagctctttgatgaggacctgaaggaattcacaaagccactctattcagacacatttttttctttacccatcactactgaatcagagcccttgtttggagctattgaacgcggccgccatcatcatcatcatcacgga

SEQ ID NO: 2 - protein, 1541 aa, Homo sapiens
MLSHGAGLALWITLSLLQTGLAEPERCNFTLAESKASSHSVSIQWRILGSPCNFSLIYSSDTLGAALCPTFRIDNTTYGCNLQDLQAGTIYNFKIISLDEERTVVLQTDPLPPARFGVSKEKTTSTGLHVWWTPSSGKVTSYEVQLFDENNQKIQGVQIQESTSWNEYTFFNLTAGSKYNIAITAVSGGKRSFSVYTNGSTVPSPVKDIGISTKANSLLISWSHGSGNVERYRLMLMDKGILVHGGVVDKHATSYAFHGLTPGYLYNLTVMTEAAGLQNYRWKLVRTAPMEVSNLKVTNDGSLTSLKVKWQRPPGNVDSYNITLSHKGTIKESRVLAPWITETHFKELVPGRLYQVTVSCVSGELSAQKMAVGRTFPLAVLQLRVKHANETSLSIMWQTPVAEWEKYIISLADRDLLLIHKSLSKDAKEFTFTDLVPGRKYMATVTSISGDLKNSSSVKGRTVPAQVTDLHVANQGMTSSLFTNWTQAQGDVEFYQVLLIHENVVIKNESISSETSRYSFHSLKSGSLYSVVVTTVSGGISSRQVVVEGRTVPSSVSGVTVNNSGRNDYLSVSWLLAPGDVDNYEVTLSHDGKVVQSLVIAKSVRECSFSSLTPGRLYTVTITTRSGKYENHSFSQERTVPDKVQGVSVSNSARSDYLRVSWVHATGDFDHYEVTIKNKNNFIQTKSIPKSENECVFVQLVPGRLYSVTVTTKSGQYEANEQGNGRTIPEPVKDLTLRNRSTEDLHVTWSGANGDVDQYEIQLLFNDMKVFPPFHLVNTATEYRFTSLTPGRQYKILVLTISGDVQQSAFIEGFTVPSAVKNIHISPNGATDSLTVNWTPGGGDVDSYTVSAFRHSQKVDSQTIPKHVFEHTFHRLEAGEQYQIMIASVSGSLKNQINVVGRTVPASVQGVIADNAYSSYSLIVSWQKAAGVAERYDILLLTENGILLRNTSEPATTKQHKFEDLTPGKKYKIQILTVSGGLFSKEAQTEGRTVPAAVTDLRITENSTRHLSFRWTASEGELSWYNIFLYNPDGNLQERAQVDPLVQSFSFQNLLQGRMYKMVIVTHSGELSNESFIFGRTVPASVSHLRGSNRNTTDSLWFNWSPASGDFDFYELILYNPNGTKKENWKDKDLTEWRFQGLVPGRKYVLWVVTHSGDLSNKVTAESRTAPSPPSLMSFADIANTSLAITWKGPPDWTDYNDFELQWLPRDALTVFNPYNNRKSEGRIVYGLRPGRSYQFNVKTVSGDSWKTYSKPIFGSVRTKPDKIQNLHCRPQNSTAIACSWIPPDSDFDGYSIECRKMDTQEVEFSRKLEKEKSLLNIMMLVPHKRYLVSIKVQSAGMTSEVVEDSTITMIDRPPPPPPHIRVNEKDVLISKSSINFTVNCSWFSDTNGAVKYFTVVVREADGSDELKPEQQHPLPSYLEYRHNASIRVYQTNYFASKCAENPNSNSKSFNIKLGAEMESLGGKCDPTQQKFCDGPLKPHTAYRISIRAFTQLFDEDLKEFTKPLYSDTFFSLPITTESEPLFGAIERGRHHHHHHG

SEQ ID NO: 3 - protein, 1621 aa, Homo sapiens
MLSHGAGLALWITLSLLQTGLAEPERCNFTLAESKASSHSVSIQWRILGSPCNFSLIYSSDTLGAALCPTFRIDNTTYGCNLQDLQAGTIYNFKIISLDEERTVVLQTDPLPPARFGVSKEKTTSTGLHVWWTPSSGKVTSYEVQLFDENNQKIQGVQIQESTSWNEYTFFNLTAGSKYNIAITAVSGGKRSFSVYTNGSTVPSPVKDIGISTKANSLLISWSHGSGNVERYRLMLMDKGILVHGGVVDKHATSYAFHGLSPGYLYNLTVMTEAAGLQNYRWKLVRTAPMEVSNLKVTNDGSLTSLKVKWQRPPGNVDSYNITLSHKGTIKESRVLAPWITETHFKELVPGRLYQVTVSCVSGELSAQKMAVGRTFPDKVANLEANNNGRMRSLVVSWSPPAGDWEQYRILLFNDSVVLLNITVGKEETQYVMDDTGLVPGRQYEVEVIVESGNLKNSERCQGRTVPLAVLQLRVKHANETSLSIMWQTPVAEWEKYIISLADRDLLLIHKSLSKDAKEFTFTDLVPGRKYMATVTSISGDLKNSSSVKGRTVPAQVTDLHVANQGMTSSLFTNWTQAQGDVEFYQVLLIHENVVIKNESISSETSRYSFHSLKSGSLYSVVVTTVSGGISSRQVVVEGRTVPSSVSGVTVNNSGRNDYLSVSWLLAPGDVDNYEVTLSHDGKVVQSLVIAKSVRECSFSSLTPGRLYTVTITTRSGKYENHSFSQERTVPDKVQGVSVSNSARSDYLRVSWVHATGDFDHYEVTIKNKNNFIQTKSIPKSENECVFVQLVPGRLYSVTVTTKSGQYEANEQGNGRTIPEPVKDLTLRNRSTEDLHVTWSGANGDVDQYEIQLLFNDMKVFPPFHLVNTATEYRFTSLTPGRQYKILVLTISGDVQQSAFIEGFTVPSAVKNIHISPNGATDSLTVNWTPGGGDVDSYTVSAFRHSQKVDSQTIPKHVFEHTFHRLEAGEQYQIMIASVSGSLKNQINVVGRTVPASVQGVIADNAYSSYSLIVSWQKAAGVAERYDILLLTENGILLRNTSEPATTKQHKFEDLTPGKKYKIQILTVSGGLFSKEAQTEGRTVPAAVTDLRITENSTRHLSFRWTASEGELSWYNIFLYNPDGNLQERAQVDPLVQSFSFQNLLQGRMYKMVIVTHSGELSNESFIFGRTVPASVSHLRGSNRNTTDSLWFNWSPASGDFDFYELILYNPNGTKKENWKDKDLTEWRFQGLVPGRKYVLWVVTHSGDLSNKVTAESRTAPSPPSLMSFADIANTSLAITWKGPPDWTDYNDFELQWLPRDALTVFNPYNNRKSEGRIVYGLRPGRSYQFNVKTVSGDSWKTYSKPIFGSVRTKPDKIQNLHCRPQNSTAIACSWIPPDSDFDGYSIECRKMDTQEVEFSRKLEKEKSLLNIMMLVPHKRYLVSIKVQSAGMTSEVVEDSTITMIDRPPPPPPHIRVNEKDVLISKSSINFTVNCSWFSDTNGAVKYFTVVVREADGSDELKPEQQHPLPSYLEYRHNASIRVYQTNYFASKCAENPNSNSKSFNIKLGAEMESLGGKRDPTQQKFCDGPLKPHTAYRISIRAFTQLFDEDLKEFTKPLYSDTFFSLPITTESEPLFGAIE

SEQ ID NO: 4 - protein, 775 aa, Homo sapiens
MLSHGAGLALWITLSLLQTGLAEPERCNFTLAESKASSHSVSIQWRILGSPCNFSLIYSSDTLGAALCPTFRIDNTTYGCNLQDLQAGTIYNFKIISLDEERTVVLQTDPLPPARFGVSKEKTTSTGLHVWWTPSSGKVTSYEVQLFDENNQKIQGVQIQESTSWNEYTFFNLTAGSKYNIAITAVSGGKRSFSVYTNGSTVPSPVKDIGISTKANSLLISWSHGSGNVERYRLMLMDKGILVHGGVVDKHATSYAFHGLTPGYLYNLTVMTEAAGLQNYRWKLVRTAPMEVSNLKVTNDGSLTSLKVKWQRPPGNVDSYNITLSHKGTIKESRVLAPWITETHFKELVPGRLYQVTVSCVSGELSAQKMAVGRTFPLAVLQLRVKHANETSLSIMWQTPVAEWEKYIISLADRDLLLIHKSLSKDAKEFTFTDLVPGRKYMATVTSISGDLKNSSSVKGRTVPAQVTDLHVANQGMTSSLFTNWTQAQGDVEFYQVLLIHENVVIKNESISSETSRYSFHSLKSGSLYSVVVTTVSGGISSRQVVVEGRTVPSSVSGVTVNNSGRNDYLSVSWLLAPGDVDNYEVTLSHDGKVVQSLVIAKSVRECSFSSLTPGRLYTVTITTRSGKYENHSFSQERTVPDKVQGVSVSNSARSDYLRVSWVHATGDFDHYEVTIKNKNNFIQTKSIPKSENECVFVQLVPGRLYSVTVTTKSGQYEANEQGNGRTIPEKGNSADIQHSGGRSSLEGPRFEGKPIPNPLLGLDSTRTGHHHHHH

SEQ ID NO: 5 - protein, 421 aa, Homo sapiens
MLSHGAGLALWITLSLLQTGLAEPERCNFTLAESKASSHSVSIQWRILGSPCNFSLIYSSDTLGAALCPTFRIDNTTYGCNLQDLQAGTIYNFKIISLDEERTVVLQTDPLPPARFGVSKEKTTSTGLHVWWTPSSGKVTSYEVQLFDENNQKIQGVQIQESTSWNEYTFFNLTAGSKYNIAITAVSGGKRSFSVYTNGSTVPSPVKDIGISTKANSLLISWSHGSGNVERYRLMLMDKGILVHGGVVDKHATSYAFHGLTPGYLYNLTVMTEAAGLQNYRWKLVRTAPMEVSNLKVTNDGSLTSLKVKWQRPPGNVDSYNITLSHKGTIKESRVLAPWITETHFKELVPGRLYQVTVSCVSGELSAQKMAVGRTFKGNSADIQHSGGRSSLEGPRFEGKPIPNPLLGLDSTRTGHHHHHH

SEQ ID NO: 6 - protein, 247 aa, Homo sapiens
MLSHGAGLALWITLSLLQTGLAEPERCNFTLAESKASSHSVSIQWRILGSPCNFSLIYSSDTLGAALCPTFRIDNTTYGCNLQDLQAGTIYNFKIISLDEERTVVLQTDPLPPARFGVSKEKTTSTGLHVWWTPSSGKVTSYEVQLFDENNQKIQGVQIQESTSWNEYTFFNLTAGSKYNIAITAVSGGKRSFSVYTNGSTVKGNSADIQHSGGRSSLEGPRFEGKPIPNPLLGLDSTRTGHHHHHH

SEQ ID NO: 7 - protein, 1632 aa, Mus musculus
MLRHGALTALWITLSVVQTGVAEQVKCNFTLLESRVSSLSASIQWRTFASPCNFSLIYSSDTSGPMWCHPIRIDNFTYGCNPKDLQAGTVYNFRIVSLDGEESTLVLQTDPLPPARFEVNREKTASTTLQVRWTPSSGKVSWYEVQLFDHNNQKIQEVQVQESTTWSQYTFLNLTEGNSYKVAITAVSGEKRSFPVYINGSTVPSPVKDLGISPNPNSLLISWSRGSGNVEQYRLVLMDKGAIVQDTNVDRRDTSYAFHELTPGHLYNLTIVTMASGLQNSRWKLVRTAPMEVSNLKVTNDGRLTSLNVKWQKPPGDVDSYSITLSHQGTIKESKTLAPPVTETQFKDLVPGRLYQVTISCISGELSAEKSAAGRTVPEKVRNLVSYNEIWMKSFTVNWTPPAGDWEHYRIVLFNESLVLLNTTVGKEETHYALDGLELIPGRQYEIEVIVESGNLRNSERCQGRTVPLAVLQLRVKHANETSLGITWRAPLGEWEKYIISLMDRELLVIHKSLSKDAKEFTFTDLMPGRNYKATVTSMSGDLKQSSSIKGRTVPAQVTDLHVNNQGMTSSLFTNWTKALGDVEFYQVLLIHENVVVKNESVSSDTSRYSFRALKPGSLYSVVVTTVSGGISSRQVVAEGRTVPSSVSGVTVNNSGRNDYLSVSWLPAPGEVDHYVVSLSHEGKVDQFLIIAKSVSECSFSSLTPGRLYNVTVTTKSGNYASHSFTEERTVPDKVQGISVSNSARSDYLKVSWVHATGDFDHYEVTIKNRESFIQTKTIPKSENECEFIELVPGRLYSVTVSTKSGQYEASEQGTGRTIPEPVKDLTLLNRSTEDLHVTWSRANGDVDQYEVQLLFNDMKVFPHIHLVNTATEYKFTALTPGRHYKILVLTISGDVQQSAFIEGLTVPSTVKNIHISANGATDRLMVTWSPGGGDVDSYVVSAFRQDEKVDSQTIPKHASEHTFHRLEAGAKYRIAIVSVSGSLRNQIDALGQTVPASVQEVVAANAYSSNSLTVSWQKALGVAERYDILLLNENGLLLSNVSEPATARQHKFEDLTPGKKYKMQILTVSGGLFSKESQAEGRTVPAAVTNLRITENSSRYLSFGWTASEGELSWYNIFLYNPDRTLQERAQVDPLVQSFSFQNLLQGRMYKMVIVTHSGELSNESFIFGRTVPAAVNHLKGSHRNTTDSLWFSWSPASGDFDFYELILYNPNGTKKENWKEKDVTEWRFQGLVPGRKYTLYVVTHSGDLSNKVTGEGRTAPSPPSLLSFADVANTSLAITWKGPPDWTDYNDFELQWFPGDALTIFNPYSSRKSEGRIVYGLHPGRSYQFSVKTVSGDSWKTYSKPISGSVRTKPDKIQNLHCRPQNSTAIACSWIPPDSDFDGYSIECRKMDTQEIEFSRKLEKEKSLLNIMMLVPHKRYLVSIKVQSAGMTSEVVEDSTITMIDRPPQPPPHIRVNEKDVLISKSSINFTVNCSWFSDTNGAVKYFAVVVREADSMDELKPEQQHPLPSYLEYRHNASIRVYQTNYFASKCAESPDSSSKSFNIKLGAEMDSLGGKCDPSQQKFCDGPLKPHTAYRISIRAFTQLFDEDLKEFTKPLYSDTFFSMPITTESEPLFGVIERGRHHHHHHG

SEQ ID NO: 8 - protein, 774 aa, Artificial
  human-mouse chimeric molecule
MLRHGALTALWITLSVVQTGVAEPERCNFTLAESKASSHSVSIQWRILGSPCNFSLIYSSDTLGAALCPTFRIDNTTYGCNLQDLQAGTIYNFKIISLDEERTVVLQTDPLPPARFGVSKEKTTSTGLHVWWTPSSGKVTSYEVQLFDENNQKIQGVQIQESTSWNEYTFFNLTAGSKYNIAITAVSGGKRSFSVYTNGSTVPSPVKDLGISPNPNSLLISWSRGSGNVEQYRLVLMDKGAIVQDTNVDRRDTSYAFHELTPGHLYNLTIVTMASGLQNSRWKLVRTAPMEVSNLKVTNDGRLTSLNVKWQKPPGDVDSYSITLSHQGTIKESKTLAPPVTETQFKDLVPGRLYQVTISCISGELSAEKSAAGRTVPEKVRNLVSYNEIWMKSFTVNWTPPAGDWEHYRIVLFNESLVLLNTTVGKEETHYALDGLELIPGRQYEIEVIVESGNLRNSERCQGRTVPLAVLQLRVKHANETSLGITWRAPLGEWEKYIISLMDRELLVIHKSLSKDAKEFTFTDLMPGRNYKATVTSMSGDLKQSSSIKGRTVPAQVTDLHVNNQGMTSSLFTNWTKALGDVEFYQVLLIHENVVVKNESVSSDTSRYSFRALKPGSLYSVVVTTVSGGISSRQVVAEGRTVPSSVSGVTVNNSGRNDYLSVSWLPAPGEVDHYVVSLSHEGKVDQFLIIAKSVSECSFSSLTPGRLYNVTVTTKSGNYASHSFTEERTKGNSADIQHSGGRSSLEGPRFEGKPIPNPLLGLDSTRTGHHHHHH

SEQ ID NO: 9 - protein, 775 aa, Artificial
  human-mouse chimeric molecule
MLRHGALTALWITLSVVQTGVAEQVKCNFTLLESRVSSLSASIQWRTFASPCNFSLIYSSDTSGPMWCHPIRIDNFTYGCNPKDLQAGTVYNFRIVSLDGEESTLVLQTDPLPPARFGVSKEKTTSTGLHVWWTPSSGKVTSYEVQLFDENNQKIQGVQIQESTSWNEYTFFNLTAGSKYNIAITAVSGGKRSFSVYTNGSTVPSPVKDLGISPNPNSLLISWSRGSGNVEQYRLVLMDKGAIVQDTNVDRRDTSYAFHELTPGHLYNLTIVTMASGLQNSRWKLVRTAPMEVSNLKVTNDGRLTSLNVKWQKPPGDVDSYSITLSHQGTIKESKTLAPPVTETQFKDLVPGRLYQVTISCISGELSAEKSAAGRTVPEKVRNLVSYNEIWMKSFTVNWTPPAGDWEHYRIVLFNESLVLLNTTVGKEETHYALDGLELIPGRQYEIEVIVESGNLRNSERCQGRTVPLAVLQLRVKHANETSLGITWRAPLGEWEKYIISLMDRELLVIHKSLSKDAKEFTFTDLMPGRNYKATVTSMSGDLKQSSSIKGRTVPAQVTDLHVNNQGMTSSLFTNWTKALGDVEFYQVLLIHENVVVKNESVSSDTSRYSFRALKPGSLYSVVVTTVSGGISSRQVVAEGRTVPSSVSGVTVNNSGRNDYLSVSWLPAPGEVDHYVVSLSHEGKVDQFLIIAKSVSECSFSSLTPGRLYNVTVTTKSGNYASHSFTEERTKGNSADIQHSGGRSSLEGPRFEGKPIPNPLLGLDSTRTGHHHHHH

SEQ ID NO: 10 - protein, 774 aa, Artificial; identical to SEQ ID NO: 8
  human-mouse chimeric molecule

(19) United States
(12) Patent Application Publication
(10) Pub. No.: US 2009/0155180 A1
(43) Pub. Date: Jun. 18, 2009

(54) Methods and targets for identifying compounds for regulating rhinovirus infection

(76) Inventors: Mary Lynn Jump, Fairfield, OH (US); Jeffrey Warren Clymer, Mason, OH (US); Begonia Y. Ho, Cincinnati, OH (US); Amy Ann Walanski, Fairfield, OH (US); Cynthia E. Francis, Alpharetta, GA (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/072,731

(22) Filed: Feb. 28, 2008

Related U.S. Application Data
(60) Provisional application No. 60/903,989, filed on Feb. 28, 2007.

(51) Int. Cl. A61K 49/00; C12Q 1/68; G01N 33/53; C12Q 1/70; C12Q 1/02; A61K 31/34; A61K 36/00; A61K 31/12; A61K 31/192; A61K 31/47; A61K 31/075; A61P 31/12

(52) U.S. Cl. 424/9.2; 435/6; 435/7.1; 435/5; 435/29; 514/473; 424/725; 514/679; 514/570; 514/311; 514/718

(57) ABSTRACT

The present invention provides methods for identifying genes, expression regulators, receptors, protein product receptors, and proteins that may regulate rhinovirus infections. The genes identified may be used as markers for disease onset and progression and to measure efficacy of a therapeutic. The present invention also provides methods to screen agents that are capable of regulating rhinovirus infection. The present invention also provides methods of identifying therapeutic compounds that may treat various disorders by regulating the expression and activity of genes, expression regulators, receptors, protein product receptors, and proteins identified.

### CROSS REFERENCE TO RELATED APPLICATIONS

This application claims the benefit of U.S. Provisional Application No. 60/903,989, filed Feb. 28, 2007.

### REFERENCE TO ELECTRONIC SUBMISSION OF A TABLE

This Application contains data tables (designated as Table I and II in the specification) as an appendix on a compact disc as required under 37 CFR §1.52(e)(1)(iii) and 37 CFR §1.58, and is herein incorporated by reference in its entirety in accordance with 37 CFR §1.77(b)(4). A duplicate disc is also provided as required under 37 CFR §1.52(e)(4). The compact disc is identical in its content. The compact disc contains a single ASCII (.doc) file for the Tables I and II, entitled “Table I and II.doc”, using an IBM-PC machine format, is 71 kb in size, and is Windows XP compatible.

### FIELD OF THE INVENTION

The present invention relates to methods of identifying target genes, proteins, expression regulators, receptors, protein product receptors, and compounds for regulating, diagnosing, and monitoring a rhinovirus infection.

### BACKGROUND OF THE INVENTION

The symptoms of the common cold are predominantly caused by 200 different viruses with rhinoviruses accounting for approximately 30-50% of colds. They are also the most prevalent pathogen associated with acute exacerbations of asthma and chronic obstructive pulmonary disease (COPD). The mechanisms by which rhinovirus triggers or exacerbates airway diseases, however, remain to be fully elucidated.

Common cold infections are so widespread that it has been estimated that adults may suffer 2-3 colds/year and children may suffer 5-7 colds/year. In the US, 50% of visits to the doctor's office are about respiratory-based illnesses. Colds are responsible for 50% of short-term absences from work and school. The average duration of a cold is 7-10 days. Effective treatment to decrease symptom severity, shorten the duration of a cold and decrease the incidence of colds has been an elusive goal. Commercial cold treatments are effective against some cold symptoms but not others.

Rhinoviruses (RV) are small non-enveloped plus-strand RNA-containing viruses that belong to the Picornavirus family. RV can be transmitted by aerosol or direct contact. Rhinovirus infection is a major cause of the common cold and yet our mechanistic understanding of how the infection leads to illness is limited.

The primary site of inoculation is the nasal mucosa. RV enters the body through the nose by attaching to the respiratory epithelium and spreads locally, traveling to the nasal pharynx. Most strains of RV enter the epithelial cells through intercellular adhesion molecule 1 (ICAM-1), the human RV receptor. RV also uses ICAM-1 for subsequent viral uncoating during cell invasion. Once in the cell, the viral replication process begins and viral shedding occurs within 8-10 hours. RV is shed in large amounts, with as many as 1 million infectious virions present per milliliter of nasal washings. Viral shedding can occur a few days before cold symptoms are recognized by the patient, peaks on days 2-7 of the illness and may last for as many as 3-4 weeks.

The pathogenesis of the common cold is complex. It has been determined that cultured human airway epithelial cells respond to infection with human rhinovirus by generating a variety of proinflammatory and host defense molecules that could play a role in disease pathogenesis. Therefore, the consensus of the experts is that the host response, not the virus, causes most symptoms of the common cold. This relationship between inflammatory mediators and cold symptoms has been studied in some detail. The cold symptoms result from the action of multiple inflammatory pathways. A local inflammatory response to the virus in the respiratory tract can lead to nasal discharge, nasal congestion, sneezing and throat irritation. Damage to the nasal epithelium does not occur and inflammation is mediated by the production of cytokines and other mediators. The generation of this complex mixture of pro-inflammatory and anti-inflammatory cytokines can occur as early as 3-8 hours post-infection. Over time, cytokine levels increase and decrease over the course of the development of cold symptoms. Cold treatments based on a single molecule approach do not block all of these pathways, only giving partial relief. This is an area in which products can be used to influence the generation of inflammatory mediators and consequently cold symptoms.

By days 3-5 of the illness, nasal discharge can become mucopurulent from polymorphonuclear leukocytes that have migrated to the infection site in response to chemoattractants such as interleukin-8. Nasal mucocilliary transport is reduced markedly during the illness and may be impaired for weeks. Both secretory immunoglobulin A and serum antibodies are involved in resolving the illness and protecting from reinfection.

Thus, there is a continuing need to identify regulators of the colds process. However, one problem associated with identification of compounds for use in the treatment of colds has been the lack of good screening targets and of screening methods for the identification of such compounds. The rapidly advancing fields of genomics and bioinformatics now offer the potential for a much more comprehensive assessment yielding greater insight into fundamental processes associated with this illness.

### SUMMARY OF THE INVENTION

The present invention relates to a method for identifying compounds for regulating rhinovirus infection, comprising: contacting at least one compound with a target selected from the group consisting of genes identified in Table I, proteins encoded by genes of Table I, expression regulators encoded by genes of Table I, receptors of proteins encoded by genes of Table I, products of proteins encoded by genes of Table I, receptors of products of proteins of genes of Table I, and combinations thereof; determining whether said compound binds the target; and identifying those compounds that bind the target as compounds for regulating rhinovirus infection.

The present invention further relates to a method for identifying compounds for regulating rhinovirus infection, comprising: contacting at least one compound with a target selected from the group consisting of genes identified in Table I, proteins identified in Table II encoded by genes of Table I, expression regulators identified in Table II of genes of Table I, receptors of proteins identified in Table II encoded by genes of Table I, products of proteins identified in Table II encoded by genes of Table I, receptors of products of proteins identified in Table II of genes of Table I, and combinations thereof; determining whether said compound binds the target; and identifying those compounds that bind the target as compounds for regulating rhinovirus infection.

The present invention further relates to a method for identifying compounds for regulating rhinovirus infection, comprising: contacting at least one compound with rhinovirus infection model system containing a target with a target selected from the group consisting of genes identified in Table I, proteins encoded by genes of Table I, expression regulators of genes of Table I, receptors of proteins encoded by genes of Table I, products of proteins encoded by genes of Table I, receptors of products of proteins of genes of Table I, and combinations thereof; further determining whether the compound regulates rhinovirus infection in an rhinovirus infection model system; and identifying those compounds that regulate rhinovirus infection in an rhinovirus infection model system as compounds for regulating rhinovirus infection.

The present invention further relates to a method for identifying compounds for regulating rhinovirus infection, comprising: contacting at least one compound with a target selected from the group consisting of genes identified in Table I, proteins identified in Table II encoded by genes of Table I, expression regulators identified in Table II of genes of Table I, receptors of proteins identified in Table II encoded by genes of Table I, products of proteins identified in Table II encoded by genes of Table I, receptors of products of proteins identified in Table II of genes of Table I, and combinations thereof; determining whether the compound binds the target; further determining whether the compound regulates rhinovirus infection in an rhinovirus infection model system; and identifying those compounds that regulate rhinovirus infection in an rhinovirus infection model system as compounds for regulating rhinovirus infection.

The present invention further relates to a method for identifying compounds for regulating rhinovirus infection, comprising: contacting at least one compound with rhinovirus infection model system containing a target with a target selected from the group consisting of genes identified in Table I, proteins encoded by genes of Table I, expression regulators of genes of Table I, receptors of proteins encoded by genes of Table I, products of proteins encoded by genes of Table I, receptors of products of proteins of genes of Table I, and combinations thereof; further determining whether the compound regulates response to rhinovirus infection in an rhinovirus infection model system; and identifying those compounds that regulates response to rhinovirus infection in an rhinovirus infection model system as compounds for regulating rhinovirus infection.

The present invention further relates to a method for identifying compounds for regulating rhinovirus infection: contacting at least one compound with a cell population expressing a protein encoded by the genes of Table I identified in Table II; determining and comparing the level of activity of the protein in the cell population that is contacted with the compound to the level of activity of the protein in the cell population that is not contacted with the compound; and identifying those compounds that modulate the activity of the protein in the cell population that is contacted with the compound compared to the activity in the cell population that is not contacted with the compound as compounds for regulating rhinovirus infection.

The present invention further relates to a method for identifying compounds for regulating rhinovirus infection, comprising: contacting at least one compound with a cell population expressing a protein identified in Table I; determining and comparing the level of activity of the protein in the cell population that is contacted with the compound to the level of activity of the protein in the cell population that is not contacted with the compound; and identifying those compounds that modulate the activity of the protein in the cell population that is contacted with the compound compared to the activity in the cell population that is not contacted with the compound as compounds for regulating rhinovirus infection.

The present invention further relates to a method for identifying compounds for regulating a rhinovirus infection, comprising: contacting at least one compound with a cell population expressing a protein encoded by genes of Table I identified in Table II; determining and comparing the level of expression of the protein in the cell population that is contacted with the compound to the level of expression of the protein in the cell population that is not contacted with the compound; and identifying those compounds that modulate the expression of the protein in the cell population that is contacted with the compound compared to the expression of the protein in the cell population that is not contacted with the compound as compounds for regulating rhinovirus infection.

The present invention further relates to a method for identifying compounds for regulating a rhinovirus infection, comprising: contacting at least one compound with a cell population expressing a protein identified in Table I; determining and comparing the level of expression of the protein in the cell population that is contacted with the compound to the level of expression of the protein in the cell population that is not contacted with the compound; and identifying those compounds that modulate the expression of the protein in the cell population that is contacted with the compound compared to the expression of the protein in the cell population that is not contacted with the compound as compounds for regulating rhinovirus infection.

The present invention further relates to a method for identifying compounds for regulating rhinovirus infection, comprising: contacting at least one compound with a cell population expressing a gene identified in Table I; determining and comparing the level of expression of the gene in the cell population that is contacted with the compound to the level of expression of the gene in the cell population that is not contacted with the compound; and identifying those compounds that modulate the expression of the gene in the cell population that is contacted with the compound compared to the expression of the gene in the cell population that is not contacted with the compound as compounds for regulating rhinovirus infection.

The present invention further relates to a method of diagnosing a rhinovirus infection, comprising: determining in a biological sample an expression profile for one or more targets selected from the group involved in rhinovirus infection identified in Tables I and Table II in a biological sample; or measuring the level of expression or activity of one or more proteins involved in regulating rhinovirus infection identified in Table II in a biological sample; comparing levels of expression of one or more target identified in a biological sample to levels of expression of one or more targets from a control sample or database, or comparing levels of expression or activity profile of the proteins from the sample to levels of expression or activity profile of the proteins from a control sample or from a database, wherein significant deviation from control levels is indicative of symptom development in rhinovirus infection.

The present invention further relates to a method of diagnosing a rhinovirus infection, comprising: preparing a gene expression profile for one or more genes involved in rhinovirus infection identified in Table I; or measuring the level of expression or activity of one or more proteins involved in regulating rhinovirus infection identified in Table I in a biological sample; comparing levels of expression of the genes from the sample to levels of expression of the genes from a control sample or database, or comparing levels of expression or activity of the proteins from the sample to levels of expression or activity of the proteins from a control sample or from a database, wherein significant deviation from control levels is indicative of symptom development in rhinovirus infection.

The present invention further relates to a method of monitoring progression of rhinovirus infection, comprising: (a) determining a gene expression profile for one or more gene involved in regulating rhinovirus infection identified in Table I in a biological sample; or preparing a protein expression profile, or protein activity profile of one or more proteins involved in regulating rhinovirus infection identified in Table I in a biological sample from a suitable rhinovirus infection model system; (b) preparing a similar expression or activity profile as in step (a) after a suitable time after the therapeutic regimen; repeating step (b) during the course of the therapy and evaluating the data to monitor progression of rhinovirus infection.

The present invention further relates to a method of monitoring progression of rhinovirus infection, comprising: (a) preparing a gene expression profile for one or more genes involved in regulating rhinovirus infection identified in Table I in a biological sample; or preparing a protein expression profile, or protein activity profile of one or more proteins involved in regulating rhinovirus infection identified in Table I from a suitable rhinovirus infection model system; (b) administering a therapeutic regimen to the subject; (c) preparing a similar expression or activity profile as in step (a) after a suitable time after the therapeutic regimen; (d) comparing the profiles prior to the intervention with profiles after the intervention; and repeating steps (b), (c) and (d) during the course of the therapy and evaluating the data to monitor progression of rhinovirus infection.

The present invention further relates to a method of monitoring the treatment or progression of a disorder in a patient with symptom development in rhinovirus infection, comprising: (a) determining a gene expression profile for one or more genes involved in regulating rhinovirus infection identified in Table I in a biological sample; or preparing a protein expression profile, or protein activity profile of one or more proteins involved in regulating rhinovirus infection identified in Table I in a biological sample from a subject; (b) administering a therapeutic regimen to the subject; (c) preparing a similar expression or activity profile as in step (a) from a biological sample from the subject after a suitable time after the therapeutic regimen; (d) comparing the profiles prior to the therapy with profiles after the therapy; and repeating steps (b), (c) and (d) during the course of the treatment or disorder and evaluating the data to monitor efficacy of the treatment or progression of the disorder.

The present invention further relates to a method of monitoring the treatment or progression of a disorder in a patient with symptom development in rhinovirus infection, comprising: (a) preparing a gene expression profile for one or more genes involved in regulating rhinovirus infection identified in Table I; or preparing a protein expression profile, or protein activity profile of one or more proteins involved in regulating rhinovirus infection identified in Table II from a subject; (b) administering a therapeutic regimen to the subject; (c) preparing a similar expression or activity profile as in step (a) from a cell or tissue sample from the subject after a suitable time after the therapeutic regimen; (d) comparing the profiles prior to the therapy with profiles after the therapy; and repeating the steps (b), (c) and (d) during the course of the treatment or disorder and evaluating the data to monitor efficacy of the treatment or progression of the disorder.

The present invention further relates to a medicinal composition, comprising: a safe and effective amount of at least one compound identified by the method of contacting at least one compound with a target selected from the group consisting of genes identified in Table I, proteins encoded by genes of Table I, expression regulators of genes of Table I, receptors of proteins encoded by genes of Table I, products of proteins encoded by genes of Table I, receptors of products of proteins of genes of Table I, and combinations thereof; determining whether the compound binds the target; and identifying those compounds that bind the target as compounds for regulating rhinovirus infection; and a pharmaceutically acceptable carrier.

The present invention further relates to a medicinal composition, comprising: a safe and effective amount of an agonist or an antagonist of a protein involved in regulating rhinovirus infection identified in Table I; and a pharmaceutically acceptable carrier.

The present invention further relates to a method for regulating rhinovirus infection in a subject in which such regulation is desirable, comprising: identifying a subject in which regulation of rhinovirus infection is desirable; and administering to the subject a safe and effective amount of compound identified by the method of: contacting at least one compound with a target selected from the group consisting of genes identified in Table I, proteins encoded by genes of Table I, expression regulators of genes of Table I, receptors of proteins encoded by genes of Table I, products of proteins encoded by genes of Table I, receptors of products of proteins of genes of Table I, and combinations thereof; determining whether the compound binds the target; and identifying those compounds that bind the target as compounds for regulating rhinovirus infection; or by the method of: contacting at least one compound with a rhinovirus infection model system containing a target with a target selected from the group consisting of genes identified in Table I, proteins encoded by genes of Table I, expression regulators of genes of Table I, receptors of proteins encoded by genes of Table I, products of proteins encoded by genes of Table I, receptors of products of proteins of genes of Table I, and combinations thereof; further determining whether the compound regulates rhinovirus infection in a rhinovirus infection model system; and identifying those compounds that regulate rhinovirus infection in a rhinovirus infection model system as compounds for regulating rhinovirus infection.

The present invention further relates to a method for regulating rhinovirus infection in a subject in which such a regulation is desirable, comprising: identifying a subject in which regulation of rhinovirus infection is desirable; and administering to the subject a safe and effective amount of compound that is an agonist, an antagonist, and activator or inhibitor of a protein from proteins encoded by the genes identified in Table I.

The nonlimiting examples of proteins, expressions regulators, products of proteins, receptors of proteins that can be encoded by the genes identified in Table I are identified in Table II.

### DETAILED DESCRIPTION OF THE INVENTION

### Molecules of the Invention

The invention comprises of various molecules: genes that are DNA; transcripts that are RNA; nucleic acids that regulate their expression such as antisense molecules, siRNAs, micro RNAs; molecules that may be used to detect them, such as DNA or RNA probes; primers that may be used to identify and isolate related genes; and proteins and polypeptides, and compounds that inhibit or activate them.

Thus, the term molecule is used herein to describe all or some of the entities of the invention. It is to be construed in the context it is used in.

Many biological functions are accomplished by altering the expression of various genes through transcriptional (e.g. through control of initiation, provision of RNA precursors, RNA processing) or translational control. For example, fundamental biological processes such as cell cycle, cell differentiation and cell death, are often characterized by the variations in the expression levels of groups of genes and their translational products.

Changes in gene expression may also be associated with pathogenesis. For example, the lack of sufficient expression of functional tumor suppressor genes or the over expression of oncogene/proto-oncogenes could lead to tumorigenesis or hyperplastic growth of cells. Thus, changes in the expression levels of particular genes or gene families may serve as signposts for the presence and progression of various diseases.

Monitoring changes in gene expression may also provide certain advantages during drug screening. Often drugs are screened for the ability to interact with a major target without regard to other effects the drugs have on cells. Often such other effects cause toxicity in the whole mammal, which prevent the use of the potential drug.

The present inventors have examined various models of rhinovirus infection to identify the global changes in gene expression during a rhinovirus infection. These global changes in gene expression, also referred to as expression profiles, may provide novel targets for the treatment of a rhinovirus infection. They may also provide useful markers for diagnostic uses as well as markers that may be used to monitor disease states, disease progression, toxicity, drug efficacy, and drug metabolism.

The expression profiles may be used to identify genes that are differentially expressed under different conditions. In addition, the present invention may be used to identify families of genes that are differentially expressed. As used herein, “gene families” includes, but is not limited to; the specific genes identified by accession numbers herein, as well as related sequences. Related sequences may be, for example, sequences having a high degree of sequence homology with an identified sequence either at the nucleotide level or at the amino acid level. A high degree of sequence homology is seen to be at least about 65% sequence identity at the nucleotide level; preferably at least about 80%, or more preferably at least about 85%, or more preferably at least about 90%, or more preferably at least about 95%, or more preferably at least about 98% or more sequence identity with an identified sequence. With regard to amino acid identity, a high degree of homology is seen to be at least about 50% sequence identity, more preferably at least about 75%, more preferably at least about 85%, more preferably at least about 95%, or more preferably at least about 98% or more sequence identity with an identified sequence. Methods are known in the art for determining homologies and identities between various sequences, some of which are described later. In particular, related sequences include homologs and orthologs from different organisms. For example, if an identified gene were from a non-human mammal, the gene family would encompass homologous genes from other vertebrates or mammals including humans. If the identified gene were a human gene, the gene family would encompass the homologous gene from different organisms. Those skilled in the art would appreciate that a homologous gene may be of different length and may comprise regions with differing amounts of sequence identity to a specifically identified sequence.

One of skill in the art would also recognize that genes and proteins from species other than those listed in the sequence listing, particularly vertebrate species, could be useful in the present invention. Such species include, but are not limited to, rats, guinea pigs, rabbits, dogs, pigs, goats, cows, monkeys, chimpanzees, sheep, hamsters and zebrafish. One of skill in the art would further recognize that by using probes from the known species' sequences, cDNA or genomic sequences homologous to the known sequence could be obtained from the same or alternate species by known cloning methods. Such homologs and orthologs are contemplated to be useful as genes and proteins of the invention.

By “variants” are intended similar sequences. For example, conservative variants may include those sequences that, because of the degeneracy of the genetic code, encode the amino acid sequence of one of the polypeptides of the invention. Naturally occurring allelic variants, and splice variants may be identified with the use of known techniques, e.g., with polymerase chain reaction (PCR), single nucleotide polymorphism (SNP) analysis, and hybridization techniques. In order to isolate orthologs and homologs, stringent hybridization conditions are generally utilized dictated by specific sequence, sequence length, guanine+cytosine (GC) content and other parameters. Variant nucleotide sequences also include synthetically derived nucleotide sequences, e.g., derived by using site-directed mutagenesis. Variants may contain additional sequences from the genomic locus alone or in combination with other sequences.

The molecules of the invention also include truncated and/or mutated proteins wherein regions of the protein not required for ligand binding or signaling have been deleted or modified. Similarly, they may be mutated to modify their ligand binding or signaling activities. Such mutations may involve non-conservative mutations, deletions, or additions of amino acids or protein domains. Variant proteins may or may not retain biological activity. Such variants may result from, e.g., genetic polymorphism or from human manipulation.

Fragments and variants of genes and proteins of the invention are also encompassed by the present invention. By “fragment” is intended a portion of the nucleotide or protein sequence. Fragments may retain the biological activity of the native protein. Fragments of a nucleotide sequence are also useful as hybridization probes and primers or to regulate expression of a gene, e.g., antisense, siRNA, or micro RNA. A biologically active portion may be prepared by isolating a portion of a nucleotide sequence, expressing the isolated portion (e.g., by recombinant expression), and assessing the activity of the encoded protein.

Fusions of a protein or a protein fragment to a different polypeptide are also contemplated. Using known methods, one of skill in the art would be able to make fusion proteins that, while different from native form, would be useful. For example, the fusion partner may be a signal (or leader) polypeptide sequence that co-translationally or post-translationally directs transfer of the protein from its site of synthesis to another site (e.g., the yeast α-factor leader). Alternatively, it may be added to facilitate purification or identification of the protein of the invention (e.g., poly-His, Flag peptide, or fluorescent proteins).

The molecules of the invention may be prepared by various methods, including, but not limited to, cloning, PCR-based cloning, site-directed mutagenesis, mutagenesis, DNA shuffling, and nucleotide sequence alterations known in the art. See, for example, Molecular Cloning: A Laboratory Manual, 2^{nd} Edition, Sambrook, Fristch, and Maniatis (1989), Cold Spring Harbor Laboratory Press; Current Protocols in Molecular Biology, Ausubel et al., (1996) and updates, John Wiley and Sons; Methods in Molecular Biology (series), volumes 158, and 182. Humana Press; PCR Protocols: A guide to Methods and Applications, Innis, Gelfand, Sninsky, and White, 1990, Academic Press.

Libraries of recombinant polynucleotides may also be generated from a population of related sequences comprising regions that have substantial sequence identity and may be recombined in vitro or in vivo. For example, using this approach, sequence motifs encoding a domain of interest may be shuffled between a gene of the invention and other known genes to obtain a new gene coding for a protein with an altered property of interest e.g. a dominant negative mutation (Ohba et al. (1998) Mol. Cell. Biol. 18:51199-51207, Matsumoto et al. (2001) J. Biol. Chem. 276:14400-14406).

The “percent identity” or “sequence identity” may be determined by aligning two sequences or subsequences over a comparison window, wherein the portion of the sequence in the comparison window may optionally comprise additions or deletions (i.e., gaps) as compared to the reference sequence (which may comprise additions or deletions) for optimal alignment of the two sequences. The percentage is calculated by determining the number of positions at which an identical residue (e.g., nucleic acid base or amino acid) occurs in both sequences, dividing the number of matched positions by the total number of positions in the window of comparison, and multiplying the result by 100 to yield the percentage of sequence identity.

Percentage sequence identity may be calculated by the local homology algorithm of Smith & Waterman, Adv. Appl. Math. 2:482-485 (1981); or by the homology alignment algorithm of Needleman & Wunsch, J. Mol. Biol. 48:443-445 (1970); either manually or by computerized implementations of these algorithms (GAP & BESTFIT in the GCG Wisconsin Software Package, Genetics Computer Group; various BLASTs from National Center for Biotechnology Information (NCBI), NIH).

A preferred method for determining homology or sequence identity is by BLAST (Basic Local Alignment Search Tool) analysis using the algorithm employed by the programs blastp, blastn, blastx, tblastn and tblastx (Karlin et al. (1990) Proc. Natl. Acad. Sci. USA 87, 2264-2268 and Altschul, (1993) J. Mol. Evol. 36, 290-300), which are tailored for sequence similarity searching.

As described herein, these various genes and proteins, their allelic and other variants (e.g. splice variants), their homologs and orthologs from other species and various fragments and mutants may exhibit sequence variations. The length of the sequence to be compared may be less than the full-length sequence.

The term “expression regulators” as used herein, unless otherwise specified, refers to a protein, DNA or other molecule that up- or down-regulate gene expression.

The term “receptors” as used herein, unless otherwise specified, refers to a receptor of the protein encoded by genes in Table I (e.g. CCR5 is the receptor of CCL5).

The term “product of protein” as used herein, unless otherwise specified, refers to product generated or mobilized by a protein enzyme encoded by genes in Table I (e.g. PGE2 is the “product of protein” of the protein COX encoded by the gene PTGE2).

The term “receptor of product of protein” as herein, unless otherwise specified, refers to receptors of the product of protein defined above (e.g. EP2 receptor for the protein product PGE2)

As used herein, the term “mammal” means a human, dog, cat, horse, cow, sheep, pig, rabbit, guinea pig, hamster, gerbil, ferret, zoo mammals, mice, and the like.

The term “binds” as herein, unless otherwise specified, refers to interacting selectively with any protein or a complex of two or more proteins that may include other nonprotein molecules; a change in state or activity of a cell or organism as a result of the perception of a stimulus; interacting selectively with any nucleic acid; playing a role in regulating transcription; combining with an extracellular or intracellular messenger to initiate a change in cell activity; and the selective, often stoichiometric, interaction of a molecule with one or more specific sites on another molecule.

Cell lines, Vectors, Cloning, and Expression of Recombinant Molecules

Molecules of the invention may be prepared for various uses, including, but not limited to: to purify a protein or nucleic acid product, to generate antibodies, for use as reagents in screening assays, and for use as pharmaceutical compositions. Some embodiments may be carried out using an isolated gene or a protein, while other embodiments may require use of cells that express them.

Where the source of molecule is a cell line, the cells may endogenously express the molecule; may have been stimulated to increase endogenous expression; or have been genetically engineered to express the molecule. Expression of a protein of interest may be determined by, for example, detection of the polypeptide with an appropriate antibody (e.g. Western blot), use of a DNA probe to detect mRNA encoding the protein (e.g., northern blot or various PCR-based techniques), or measuring binding of an agent selective for the polypeptide of interest (e.g., a suitably-labeled selective ligand).

The present invention further provides recombinant molecules that contain a coding sequence of, or a variant form of, a molecule of the invention. In a recombinant DNA molecule, a coding DNA sequence is operably linked to other DNA sequences of interest including, but not limited to, various control sequences for integration, replication, transcription, expression, and modification.

The choice of vector and control sequences to which a gene sequence of the present invention is operably linked depends upon the functional properties desired (e.g., protein expression, the host cell to be transformed). A vector of the present invention may be capable of directing the replication or insertion into the host chromosome, and preferably expression of the gene.

Control elements that are used for regulating the expression of a gene are known in the art and include, but are not limited to, inducible or constitutive promoters, secretion signals, enhancers, termination signals, ribosome-binding sites, and other regulatory elements. Optimally, the inducible promoter is readily controlled, such as being responsive to a nutrient, or an antibiotic.

In one embodiment, the vector harboring a nucleic acid molecule may include a prokaryotic replicon, i.e., a DNA sequence having the ability to direct autonomous replication and maintenance of the recombinant DNA molecule extra-chromosomally in a prokaryotic host cell, such as a bacterial host cell. In addition, vectors that include a prokaryotic replicon may also include a gene whose expression confers a detectable characteristic (e.g., resistance to ampicillin).

Vectors may further include a prokaryotic or bacteriophage promoter capable of directing the expression (transcription and translation) of the coding gene sequences in a bacterial host cell, such as E. coli. Promoter sequences compatible with bacterial hosts may be provided in plasmid vectors containing convenient restriction sites for insertion of a DNA sequence of the present invention, e.g., pcDNA1, pcDNA3.

Expression vectors compatible with eukaryotic cells may also be used to form a recombinant molecule that contains a sequence of interest. Commercially available vectors often contain both prokaryotic and eukaryotic replicons and control sequences, for an easy switch from prokaryotic to eukaryotic cell to ES cells for generating transgenic cells or mammals (e.g., pcDNA series from Invitrogen™).

Eukaryotic cell expression vectors used to construct the recombinant molecules of the present invention may further include a selectable marker that is effective in a eukaryotic cell (e.g., neomycin resistance). Alternatively, the selectable marker may be present on a separate plasmid, the two vectors introduced by co-transfection of the host cell, and transfectants selected by culturing in the appropriate drug for the selectable marker. Vectors may also contain fusion protein, or tag sequences that facilitate purification or detection of the expressed protein.

The present invention further provides host cells transformed with a recombinant molecule of the invention. The host cell may be a prokaryote, e.g., a bacterium, or a eukaryote, e.g., yeast, insect or vertebrate cells, including, but not limited to, cells from a mouse, monkey, frog, human, rat, guinea pig, rabbit, dog, pig, goat, cow, chimpanzee, sheep, hamster or zebrafish. Commonly used eukaryotic host cell lines include, but are not limited to, CHO cells, ATCC CCL61, NIH-3T3, and BHK cells. In many instances, primary cell cultures from mammals may be preferred.

Transformation of appropriate host cells with a molecule of the present invention may be accomplished by known methods that depend on the host system employed. For transforming prokaryotic host cells, electroporation and salt treatment methods may be employed, while for transformation of eukaryotic cells, electroporation, cationic lipids, or salt treatment methods may be employed (See Sambrook et al. (1989) supra). Viral vectors, including, but not limited to, retroviral and adenoviral vectors have also been developed that facilitate transfection of primary or terminally differentiated cells. Other techniques may also be used that introduce DNA into cells e.g., liposome, gold particles, or direct injection of the DNA expression vector (as a projectile), containing the gene of interest, into human tissue.

Successfully transformed cells may be cloned to produce stable clones. Cells from these clones may be harvested, lysed and their content examined for the presence of the recombinant molecules using known methods.

### Biological Samples

As is apparent to one of ordinary skill in the art, nucleic acid samples, which may be DNA and/or RNA, used in the methods and assays of the invention may be prepared by available methods. Methods of isolating total mRNA are known. For example, methods of isolation and purification of nucleic acids are described in detail in Chapter 3 of Tijssen, (1993) Laboratory Techniques in Biochemistry and Molecular Biology: Hybridization with Nucleic Acid Probes, Elsevier Press. Such samples include RNA samples, but may also include cDNA synthesized from an mRNA sample isolated from a cell or tissue of interest. Such samples also include DNA amplified from the cDNA, and RNA transcribed from the amplified DNA.

Biological samples containing nucleic acids, or proteins may be of any biological tissue or fluid or cells from any organism as well as cells grown in vitro, such as cell lines and tissue culture cells. The sample may be a “clinical sample” which is a sample derived from a patient. Typical clinical samples include, but are not limited to, sputum, nasal lavage, blood, blood-cells (e.g., white cells), various tissues or organs or parts thereof, or fine needle biopsy samples, urine, peritoneal fluid, and pleural fluid, or cells therefrom. Biological samples may also include sections of tissues, such as frozen sections or formaldehyde-fixed sections taken for histological purposes.

### Nasal Lavage Methodology

Nasal lavage samples may be collected by instillation of 5 mL of saline solution into each nostril. This wash may be immediately expelled into a waxed paper cup, kept chilled and processed in preparation for analyses.

For evaluation of presence/absence of virus and rhinovirus, a portion of the nasal lavage sample may be mixed with 4× concentrated viral collecting broth. Approximately 2 mL of the processed sample may be placed in a screw-capped cryovial and stored frozen at −70° C. until evaluation. For evaluation of biomarker concentration, a portion of the nasal lavage sample may be mixed with 5% bovine albumin. Then one (1) mL of the processed sample may be placed in a 2-mL cryovial and stored frozen at −70° C. until evaluation.

### Nasal Scraping Methodology

Nasal scraping samples may be collected from the anterior portion of the inferior turbinate under direct visualization. They may be collected by gently scraping the surface of the turbinate five times with a disposable cytology collection curette (Rhinoprobe®, Arlington Scientific, Inc., Springville, Utah). This procedure is then repeated with a second curette.

Both curettes may be placed into an RNase-free screw-capped cryovial containing TRIzol® Reagent (Invitrogen Corp., Carlsbad, Calif.) to preserve RNA. The cryovials may be vortexed to remove the cellular material from the curettes and then stored frozen at −70° C. for assay of gene expression levels.

### Gene Chip Analysis

Total RNA Isolation may include the suspension of cells in ˜500 ul of RNA-STAT60 (Tel-Test, Friendswood, Tex.) and homogenization in a Retsch (Wunsiedel, Bavaria) MM300 Bead-Beater Mill using 5 mm stainless steel beads. Chloroform is added to the lysate and the mixture is shaken for 1-2 minutes. The aqueous phase, containing crude nucleic acids, is removed and precipitated in isopropanol. Nucleic acids are pelleted by centrifugation and the pellets are washed with 70% ethanol and then resuspended in DEPC-water. RNA is then purified using QIAgen (Hilden, Germany) RNEasy Cleanup minicolumns and the manufacturer's recommended protocol. Quantity of RNA is determined by UV spectroscopy and quality is determined using an Agilent (Palo Alto, Calif.) Bioanalyzer 2100.

GeneChip Target Synthesis and GeneChip processing may involve converting purified total RNA to cRNA GeneChip targets using the protocol provided by Affymetrix. The cRNA targets are fragmented and hybridized, washed, and scanned according to the Affymetrix Expression Analysis protocol. Complete protocols for target synthesis and GeneChip processing can be found at: www.affymetrix.com/support/download/manuals/expression s2_manual.pdf

Finally, GeneChip Analysis involving GeneChip scans may be converted to tabular data using the Affymetrix MAS5.0 algorithm, which is described in: www.affymetrix.com/Auth/support/downloads/manuals/mas_manual.zip. Once the data quality is confirmed, the data may be analyzed and visualized using a variety of commercially-available tools, including Affymetrix Data Mining Tool (DMT), Spotfire (Sommerville, Mass.), and Omniviz (Maynard, Mass.).

### Isolation of Other Related Nucleic Acid Molecules

As described above, the identification of the human nucleic acid molecules of Table I and/or Table II allows a skilled artisan to isolate nucleic acid molecules that encode other members of the gene family in addition to the sequences herein described. Further, the presently disclosed nucleic acid molecules allow a skilled artisan to isolate nucleic acid molecules that encode other members of the gene families.

A skilled artisan may use the proteins of Table II or fragments thereof to generate antibody probes to screen expression libraries prepared from appropriate cells. In one embodiment, the fragments may contain amino acid insertions and substitutions. Polyclonal antiserum from mammals such as rabbits immunized with the purified protein, or monoclonal antibodies may be used to probe a mammalian cDNA or genomic expression library, such as lambda gt11 library, to obtain the appropriate coding sequence for other members of the protein family. The cloned cDNA sequence may be expressed as a fusion protein, expressed using its own control sequences, or expressed by constructs using control sequences appropriate to the particular host used for expression of a protein.

Alternatively, a portion of coding sequences herein described may be synthesized and used as a probe to retrieve DNA encoding a member of the protein family from any organism. Oligomers, e.g., containing 18-20 nucleotides, may be prepared and used to screen genomic DNA or cDNA libraries to obtain hybridization under stringent conditions or conditions of sufficient stringency to eliminate an undue level of false positives.

Additionally, pairs of oligonucleotide primers may be prepared for use in a polymerase chain reaction (PCR) to clone a nucleic acid molecule. Various PCR formats are known in the art and may be adapted for use in isolating other nucleic acid molecules.

### Selection of Test Compounds

Compounds that may be screened in accordance with the assays of the invention include, but are not limited to, libraries of known compounds, including natural products, such as plant or mammal extracts. Also included are synthetic chemicals, biologically active materials, e.g., proteins, nucleic acids, and peptides, including, but not limited to, members of random peptide libraries and combinatorial chemistry derived molecular libraries made of D- or L-configuration amino acids, and phosphopeptides, antibodies (including, but not limited to, polyclonal, monoclonal, chimeric, human, anti-idiotypic or single chain antibodies, and Fab, F(ab′)₂ and Fab expression library fragments, and epitope-binding fragments thereof); and other organic and inorganic molecules.

In addition to the more traditional sources of test compounds, computer modeling and searching technologies permit the rational selection of test compounds by utilizing structural information from the ligand binding sites of proteins of the present invention. Such rational selection of test compounds may decrease the number of test compounds that must be screened in order to identify a therapeutic compound. Knowledge of the protein sequences of the present invention may allow for generation of models of their binding sites that may be used to screen for potential ligands. This process may be accomplished by methods known in the art. A preferred approach involves generating a sequence alignment of the protein sequence to a template (derived from the crystal structures or NMR-based model of a similar protein(s)), conversion of the amino acid structures and refining the model by molecular mechanics and visual examination. If a strong sequence alignment cannot be obtained then a model may also be generated by building models of the hydrophobic helices. Mutational data that point towards contact residues may also be used to position the helices relative to each other so that these contacts are achieved. During this process, docking of the known ligands into the binding site cavity within the helices may also be used to help position the helices by developing interactions that would stabilize the binding of the ligand. The model may be completed by refinement using molecular mechanics and loop building using standard homology modeling techniques. General information regarding modeling may be found in Schoneberg, T. et. al., Molecular and Cellular Endocrinology, 151:181-193 (1999), Flower, D., Biochim Biophys Acta, 1422, 207-234 (1999), and Sexton, P. M., Curr. Opin. Drug Discovery and Development, 2, 440-448 (1999).

Once the model is completed, it may be used in conjunction with one of several computer programs to narrow the number of compounds to be screened, e.g., the DOCK program (UCSF Molecular Design Institute, 533 Parnassus Ave, U-64, Box 0446, San Francisco, Calif. 94143-0446) or FLEXX (Tripos Inc., 1699 South Hanley Rd., St. Louis, Mo.). One may also screen databases of commercial and/or proprietary compounds for steric fit and rough electrostatic complementarity to the binding site.

### Screening Assays to Identify Compounds

The finding that the genes of the present invention may play a role in regulating, monitoring and/or treating a rhinovirus infection enables various methods of screening one or more compounds to identify compounds that may be used for prophylactic or therapeutic treatment of a rhinovirus infection.

When selecting compounds useful for prevention, monitoring or treatment, it may be preferable that the compounds be selective for protein expressions regulators, products of proteins, and receptors of proteins of the present invention. For initial screening, it may be preferred that the in vitro screening be carried out using a protein of the invention with an amino acid sequence that is, e.g., at least about 80% identical, preferably at least about 90% identical, and more preferably identical to the sequence of a protein described in Table II. Preferably, the test compounds may be screened against a vertebrate protein, more preferably a human protein. For screening compounds it may be preferable to use the protein from the species in which treatment is contemplated.

The methods of the present invention may be amenable to high throughput applications; however, use of as few as one compound in the method is encompassed by the term “screening”. This in vitro screening provides a means by which to select a range of compounds, i.e., the compounds, which merit further investigation. For example, compounds that activate a protein of the invention at concentrations of less than 200 nM might be further tested in a mammal model, whereas those above that threshold may not be further tested.

The assay systems described below may be formulated into kits comprising a protein of the invention or cells expressing a protein of the invention, which may be packaged in a variety of containers, e.g., vials, tubes, microtitre plates, bottles and the like. Other reagents may be included with the kit, e.g., positive and negative control samples, and buffers.

In one embodiment, the invention provides a method to identify compounds that bind to a protein of the invention. Methods to determine binding of a compound to a protein are known in the art. The assays include incubating a protein of the invention with a labeled compound, known to bind to the protein, in the presence or absence of a test compound and determining the amount of bound labeled compound. The source of a protein of the invention may either be cells expressing the protein or some form of isolated protein. The labeled compound may be a known ligand or a ligand analog labeled such that it may be measured, preferably quantitatively (e.g., labeled with ¹²⁵I, ³⁵S-methionine, or a fluorescent tag, or peptide or a fluorescent protein fusion). Such methods of labeling are known in the art. Test compounds that bind to a protein of the invention may reduce ligand bound to the protein, thereby reducing the signal level compared to control samples. Variations of this technique have been described Keen, M., Radioligand Binding Methods for Membrane Preparations and Intact cells in Receptor Signal Transduction Protocols, R. A. J. Challis, (ed), Humana Press Inc., Totoway N.J. (1997).

In another embodiment, the invention provides methods for screening test compounds to identify compounds that activate a protein of the invention. The assays are cell-based; however, cell-free assays are known which are able to differentiate agonist and antagonist binding. Cell-based assays include contacting cells that express a protein of the invention with a test compound or a control substance and measuring activation of the protein by measuring the expression or activity of components of the affected signal transduction pathways. For example, after suitable incubation with a test compound, lysates of the cells may be prepared and assayed for transcription, translation, or modification of a protein, e.g., phosphorylation, or glycosylation, or induction of second messengers like cAMP. In addition, many high-throughput assays are available that measure the response without the need of lysing the cells, e.g. calcium imaging.

In one embodiment, cAMP induction may be measured with the use of recombinant constructs containing the cAMP responsive element linked to any of a variety of reporter genes. Such reporter genes include, but are not limited to, chloramphenicol acetyltransferase (CAT), luciferase, glucuronide synthetase, growth hormone, fluorescent proteins, or alkaline phosphatase. Following exposure of the cells to a test compound, the level of reporter gene expression may be quantified to determine the test compound's ability to increase cAMP levels and thus determine a test compound's ability to activate a protein of the invention.

In another embodiment, specific phospho-tyrosine or phospho-serine antibodies may be utilized to measure the level of phosphorylation of a signaling protein after the exposure to a test compound, whereby a significant deviation in phosphorylation levels compared to control samples would indicate activation of a protein of the invention. In some instances, a protein's (for example receptor) responses subside, or become desensitized, after prolonged exposure to an agonist. In many cases, the protein of interest may be an enzyme and thus the effect of the binding of the test compounds could be measured in terms of changes in the enzymatic activity. Similarly, changes in intracellular calcium concentration [Ca²⁺] are generally indicative of activation of many signaling cascades.

### Cell-Based Receptor Binding Assays

Cell-based receptor binding assays are commonly used in the pharmaceutical and biotechnology communities as valuable tools to assess the potential biological activities of novel compounds. In fact, this high-throughput screening (HTS) methodology has become the main source of new lead compounds for drug development. Drug discovery and basic research programs require more rapid and reliable procedures to process and screen large numbers of unknown compounds for activity. Several specialized detection technologies have been developed to facilitate the cost- and time-efficient screening of millions of compounds.

One of the most frequently used assay techniques may be scintillation proximity assay (SPA). This may be used to determine the affinity of various drugs for a receptor as well as the binding site density of receptor families and their subtypes in different tissues or samples. Inhibitors may decrease the specific chemiluminescence or radioactive intensity by competing with binding sites of the receptors. These studies may help to determine whether a drug will have therapeutic or adverse effects at different subtypes.

The general assay procedure involves adding cells or cell membranes with desired target receptors to assay plates. A blocker to minimize non-specific binding may be added and incubated for 30 minutes at RT (room temperature). Test compounds, reagents, labelled ligand, together with reading buffer may be added and incubated for a determined period of time. Readings of intensity may be taken as frequently as needed. Cells not expressing the receptor will display no specific binding. Competition binding curves may produce a rank order of potency for tested compounds.

### NF-κB Activation Assays

The transcription of many pro-inflammatory agents (e.g., cytokines, chemokines and cyclooxygenase) are regulated by the transcriptional factor NF-κB. The findings of the present inventors that both NF-κB and many chemokines and cytokines are upregulated after rhinovirus (RV) infection indicate that inhibition of NF-κB would be a key intervention point for symptom relief.

Nuclear factor-κB (NF-κB) is a key nuclear transcription factor that regulates the expression of a large number of genes critical for inflammation, including cytokine and chemokine transcription. Upon activation, NF-κB translocates from the cytoplasm to the nucleus and activates its promoter for transcription. Results from the literature and the present inventors' laboratory both support the transcription of a large number of genes after rhinovirus infection, indicating that NF-κB is a potential key intervention point. Therefore, an assay for monitoring the activation and translocation of NF-κB would be useful in assessing the anti-inflammatory potential of technologies.

Cellomics, Inc (Pittsburgh, Pa.) has developed an antibody-based assay that reveals the subcellular localization of NF-κB, thus allowing the quantification of translocation of NF-κB from the cytoplasm to the nucleus. Because NF-κB must be in the nucleus to induce gene expression, its translocation is a definitive measure of its activation and marks an earlier event than reporter gene expression. This assay is an example of a 96-well medium throughput technology that can detect NF-κB translocation in several cell types. This cell-based assay has the potential of predicting respiratory benefits.

Assays may be performed in standard, high-density microplates, where measurements of the rate and extent of NF-κB translocation are made in intact cells which provides more biological representative information. Cellomics' NF-κB activation kit (Cat. No. K01-001-1) may combine fluorescent reagents and protocols for optimized sample preparation and assays, and requires no cell lysis, purification or filtration steps. After fixation, the plates are stable for extended periods, when stored light-protected at 4° C.

One may create a fully automated screen to identify compounds that inhibit or activate NF-□B on a cell-by-cell basis. Prepared cells can be analyzed using standard fluorescence microscopy or using Cellomics' fully automated HCS Reader with the Cytoplasm to Nucleus Translocation Bioapplication, affording automated plate handling, focusing, image acquisition, analysis, quantification, and data storage.

### COX Inhibitor Screening Assay

Cyclooxygenase (COX, also called Prostaglandin H Synthase or PGHS) enzymes contain both cyclooxygenase and peroxidase activities. COX catalyzes the first step in the biosynthesis of prostaglandins (PGs), thromboxanes, and prostacyclins; the conversion of arachidonic acid to PGH2. It is now well established that there are two distinct isoforms of COX. Cyclooxygenase-1 (COX-1) is constitutively expressed in a variety of cell types and is involved in normal cellular homeostasis. A variety of mitogenic stimuli such as phorbol esters, lipopolysaccharides, and cytokines lead to the induced expression of a second isoform of COX, cyclooxygenase-2 (COX-2). COX-2 is responsible for the biosynthesis of PGs under acute inflammatory conditions. This inducible COX-2 is believed to be the target enzyme for the anti-inflammatory activity of nonsteroidal anti-inflammatory drugs.

An example of a COX Inhibitor Screening Assay (Cat. No. 560101 manufactured by Cayman Chemical Company, Ann Harbor, Mich.) directly measures PGF2 produced by SnCl2 reduction of COX-derived PGH2. The prostanoid product may be quantified via enzyme immunoassay (EIA) using a broadly specific antibody that binds to all the major prostaglandin compounds. Thus, the COX assay is more accurate and reliable than an assay based on peroxidase inhibition. The Cayman COX Inhibitor Screening Assay includes both ovine COX-1 and human recombinant COX-2 enzymes in order to screen isozyme-specific inhibitors. This assay may be an excellent tool which can be used for general inhibitor screening, or to eliminate false positive leads generated by less specific methods.

### Prostaglandin E2 Assays

Cycloxygenases can participate in the production of prostaglandins which can be mediators of inflammation and pain. COX2 (Cyclooxygenase-2) is a protein (encoded by the gene PTGS2) induced by viral infection and PGE2 (prostaglandin E2) is the product that can result in symptoms like malaise, headache, sore throat. A compound that suppresses PGE2 production or COX activity can relieve symptoms of viral infections.

The production of prostaglandins begins with the liberation of arachidonic acid from membrane phospholipids by phospholipase A2 in response to inflammatory stimuli. The cyclooxygenases enzymes COX-1 and COX-2 then convert arachidonic acid to PGH2 (Prostaglandin H2). COX-1 is expressed constitutively and acts to maintain homeostatic function such as mucus secretion, whereas COX-2 is induced in response to an inflammatory stimuli. Further downstream, cell-specific prostaglandin synthases convert PGH2 into a series of prostaglandins including PGI2, PGF2, PGD2 and PGE2. PGE2, a primary product of arachidonic acid metabolism, is produced by several cell types including macrophages, fibroblasts and some malignant cells. It exerts its actions through 4 receptors: EP1, EP2, EP3 and EP4. Its production is a commonly used method for the detection of COX-1 and COX-2 modulation and prostaglandin synthases.

There are several standard methods available for quantifying PGE2. The HTRF® PGE2 assay (developed by Cisbio International, Cat. No. 62P2APEB) is an example of a highly sensitive method for quantifying PGE2. Its principle is based on HTRF technology (Homogeneous Time-Resolved Fluorescence). It can be performed either in cell supernatants or directly in the presence of whole cells. This method is a competitive immunoassay in which native PGE2 produced by cells, and d2-labelled PGE2 compete for binding to MAb anti-PGE2 labeled with cryptate. The HTRF signal is inversely proportional to the concentration of PGE2 in the calibrator or in the sample. The incubation time and temperature following addition of the HTRF detection reagents has little effect on the assay results providing another level of assay flexibility.

Briefly, consecutive dilutions (within the 0-5000 pg/ml range) of samples may be prepared with the diluent. The reagents are dispensed (as outlined in the protocol) into a 384-well low volume plate (20 ul). Negative and positive controls are included. The plate is covered with a plate sealer and incubated for 5 hours at room temperature or overnight at 4° C. Free PGE2 from the sample competes with XL665 labeled PGE2 for binding to the Cryptate conjugated anti-PGE2 antibody. Then the plate is read on a compatible HTRF reader.

### Screening for Compounds Using Cell Culture, Tissue, and Mammal Models of Rhinovirus

Compounds selected from one or more test compounds by an in vitro assay, as described above, may be further tested for their ability to regulate rhinovirus. Such models include both in vitro cell culture models and in vivo mammal models. Such additional levels of screening are useful to further narrow the range of candidate compounds that merit additional investigation, e.g., clinical trials. Such model systems may include, but are not limited to bronchial epithelial cell prostaglandin and chemokine release assay, PBMC proliferation/survival assays, PBMC chemotaxis assays, chemokine receptor binding assays, rhinovirus tittering in RV-infected bronchial epithelial cells, and human RV-induced cold model.

### Chemotaxis Assay

Multiple chemokines in Table I are induced upon RV infection (e.g., IP10, MCP1). Chemokines are small proteins that are released by infected cells and act on receptors on other immune cells (e.g., lymphocytes) and induce chemotaxis, thus starting the inflammatory process. Therefore, viral infection can be controlled by actives that 1) down-regulate the chemokines; or 2) block the chemokine receptors. Chemokine receptor antagonists can be identified by chemotaxis assay.

The purpose of a chemotaxis assay is to determine whether a protein or small molecule of interest has chemotactic activity on a specific cell type. Chemotaxis is the ability of a protein to direct the migration of a specific cell. This assay is based on the premise of creating a gradient of the chemotactic agent and allowing cells to migrate through a membrane towards the chemotactic agent. If the agent is not chemotactic for the cell, then the majority of the cells will remain on the membrane. If the agent is chemotactic, then the cells will migrate through the membrane and settle on the bottom of the well of the chemotaxis plate.

This assay may use multi-well chambers (e.g. NeuroProbe), where 24, 96, 384 samples of leukocytes or other migratory cells are evaluated in parallel. The advantage is that several parallels are assayed in identical conditions. The multi-well chambers are separated by a filter containing pores of uniform size. Size of the leukocytes to be investigated determines the pore size of the filter. It is essential to choose a diameter which allows an active transmigration.

A solution containing a chemokine or chemotactic factor is placed in the bottom chamber and a cell suspension of leukocytes is placed in the upper chamber. The cells can migrate through the pores, across the thickness of the filter, and toward the source of chemoattractant (the lower chamber). Cells that migrated across the filter and attached to the underside are counted. Data is often expressed in terms of Migration Index: the number of cells that migrated in response to agonist relative to the number of cells that migrated randomly, that is, to buffer only. For detection of cells general staining techniques (e.g. trypan blue) or special probes (e.g. mt-dehydrogenase detection with MTT assay) are used. Labeled (e.g. fluorochromes like Cell Tracker Green) cells are also used.

### Multiplex Assay Analysis of Inflammatory Mediators

Multiplex assays have become highly useful tools for measuring the levels and/or activities of multiple proteins in a single sample. They are quantitative, plate-based antibody arrays based on traditional ELISA (Enzyme-Linked ImmunoSorbent Assay) technique and piezoelectric printing technology. They can be optimized for the quantitative measurement of multiple analytes (proteins) in serum; EDTA, heparin, and sodium citrate plasma; culture supernatants; and other sample types.

Each well of the microplate provided is pre-spotted with antibodies that capture specific analytes in standards and samples added to the plate. After non-bound proteins are washed away, the biotinylated detecting antibodies are added and bind to a second site on the target proteins. Each antibody spot may capture a specific cytokine, chemokine or other biomarker detected with a biotinylated antibody cocktail followed by addition of streptavidin-horseradish peroxidase (SA-HRP) and SuperSignal ELISA Chemiluminescent Substrate. Excess detecting antibody may be removed and SA-HRP or SA-DyLight 800 Fluor may be added. The enzyme-substrate (HRP-SuperSignal) reaction produces a luminescent signal that may be detected with the SearchLight Plus CCD Imaging System. For infrared arrays, signal from the DyLight 800 Fluor may be measured with the Odyssey® or Aerius® Infrared Imaging Systems from LI-COR Biosciences. The amount of signal produced in each spot is proportional to the amount of each specific protein in the original standard or sample.

The light produced from the HRP-catalyzed oxidation of the substrate may be measured by imaging the plate with a cooled CCD camera. Standard curves are generated using a mixture of the recombinant array proteins. Protein concentrations in a sample may be quantified by comparing the intensity of the spots to the corresponding standard curve.

### Transgenic Mammals and Gene Therapy

Mammals of many species, preferably vertebrates, including, but not limited to, mice, rats, rabbits, guinea pigs, pigs, goats, dogs, frogs, and non-human primates may be used to generate transgenic mammals expressing the proteins of the invention. Several techniques are known in the art and may be used to introduce transgenes into mammals to produce the founder lines of transgenic mammals. Such techniques include, but are not limited to, pronuclear microinjection, retrovirus-mediated gene transfer into germ lines, gene targeting in embryonic stem cells, electroporation of embryos and sperm-mediated gene transfer.

The overall activity of a protein of the invention may be increased by over-expressing the gene for that protein. Over-expression will increase the total cellular protein activity, and thereby the function. The gene or genes of interest are inserted into a vector suitable for expression in the subject. These vectors include, but are not limited to, adenoviruses, adenovirus associated viruses, retroviruses and herpes virus vectors. Other techniques may also be used that introduce DNA into cells e.g., liposome, gold particles, or direct injection of the DNA expression vector (as a projectile), containing the gene of interest, into human tissue.

### Treatment of Rhinovirus Infection

The genes, proteins, expression regulators, products of proteins, and receptors of the present invention (targets), and one or more compounds, including but not limited to at least one compound, at least two compounds, at least three compounds that activate or inhibit the targets may be used in a method for the treatment of a rhinovirus infection. The term “regulate” includes, but is not limited to, up-regulate or down-regulate, to fix, to bring order or uniformity, to govern, or to direct by various means. In one aspect, a compound may be used in a method for the treatment of a “rhinovirus infection”. Non-limiting examples of rhinovirus infection and disorders associated with rhinovirus infection that may be treated by the present invention are herein described below.

Targets and compounds of present invention may be used to treat, monitor or diagnose upper respiratory tract infections (URIs), including and not limited to colds and flus. This includes and is not limited to rhinoviruses, parainfluenza viruses, coronaviruses, adenoviruses, myxoviruses, echoviruses, respiratory syncytial viruses, coxsackieviruses, and influenza viruses which account for most URIs.

### Pharmaceutical Formulations and Methods for Use

Compounds identified by screening methods described herein may be administered to mammals to treat or to prevent diseases or disorders that are regulated by genes, proteins, expression regulators, protein products, and receptors (targets), of the present invention. The term “treatment” is used herein to mean that administration of a compound of the present invention mitigates a disease or a disorder in a host. Thus, the term “treatment” includes, preventing a disorder from occurring in a host, particularly when the host is predisposed to acquiring the disease, but has not yet been diagnosed with the disease; inhibiting the disorder; and/or alleviating or reversing the disorder. Insofar as the methods of the present invention are directed to preventing disorders, it is understood that the term “prevent” does not require that the disease state be completely thwarted. (See Webster's Ninth Collegiate Dictionary.) Rather, as used herein, the term preventing refers to the ability of the skilled artisan to identify a population that is susceptible to disorders, such that administration of the compounds of the present invention may occur prior to onset of a disease. The term does not imply that the disease state be completely avoided. The compounds identified by the screening methods of the present invention may be administered in conjunction with other compounds.

Safety and therapeutic efficacy of compounds identified may be determined by standard procedures using in vitro or in vivo technologies. Compounds that exhibit large therapeutic indices may be preferred, although compounds with lower therapeutic indices may be useful if the level of side effects is acceptable. The data obtained from the in vitro and in vivo toxicological and pharmacological techniques may be used to formulate ranges of doses.

Effectiveness of a compound may further be assessed either in mammal models or in clinical trials of patients with rhinovirus infections.

As used herein, “pharmaceutically acceptable carrier” is intended to include all solvents, dispersion media, coatings, antibacterial and antifungal agents, isotonic and absorption delaying agents, and the like, compatible with pharmaceutical administration. The use of such media and agents for pharmaceutically active substances is known in the art. Except insofar as any conventional media or agent is incompatible with the active compound, such media may be used in the compositions of the invention. Supplementary active compounds may also be incorporated into the compositions. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application may include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH may be adjusted with acids or bases, such as hydrochloric acid or sodium hydroxide. The parenteral preparation may be enclosed in ampoules, disposable syringes or multiple dose vials made of glass or plastic.

Pharmaceutical compositions suitable for injectable use include sterile aqueous solutions (where water-soluble), or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, polyol (for example, glycerol, propylene glycol, and liquid polyethylene glycol, and the like), and suitable mixtures thereof. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions may be brought about by including in the composition an agent that delays absorption, for example, aluminum monostearate and gelatin.

Sterile injectable solutions may be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They may be enclosed in gelatin capsules or compressed into tablets. For oral administration, the agent may be contained in enteric forms to survive the stomach or further coated or mixed to be released in a particular region of the GI tract by known methods. For the purpose of oral therapeutic administration, the active compound may be incorporated with excipients and used in the form of tablets, troches, or capsules. Oral compositions may also be prepared using a fluid carrier for use as a mouthwash, wherein the compound in the fluid carrier is applied orally and swished and expectorated or swallowed. Pharmaceutically compatible binding agents, and/or adjuvant materials may be included as part of the composition. The tablets, pills, capsules, troches and the like may contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration may also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration may be accomplished using nasal sprays or suppositories. For transdermal administration, the active compounds are formulated into ointments, salves, gels, or creams as generally known in the art.

The compounds may also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid. Methods for preparation of such formulations will be apparent to those skilled in the art. The materials may also be obtained commercially from Alza Corporation and Nova Pharmaceuticals, Inc. Liposomal suspensions (including liposomes targeted to infected cells with monoclonal antibodies to viral antigens) may also be used as pharmaceutically acceptable carriers. These may be prepared according to methods known to those skilled in the art, for example, as described in U.S. Pat. No. 4,522,811.

It is especially advantageous to formulate oral or parenteral compositions in dosage unit form for ease of administration and uniformity of dosage. “Dosage unit form” as used herein refers to physically discrete units suited as unitary dosages for the subject to be treated, each unit containing a predetermined quantity of active compound calculated to produce the desired therapeutic effect in association with the required pharmaceutical carrier. The specification for the dosage unit forms are dictated by and are directly dependent on the unique characteristics of the active compound and the particular therapeutic effect to be achieved, and the limitations inherent in the art of compounding such an active compound for the treatment of individuals.

### Diagnostic Uses

As described above, the genes and gene expression information provided in Table I may be used as diagnostic markers for the prediction or identification of the disease state of a sample tissue. For instance, a tissue sample may be assayed by any of the methods described above, and the expression levels for a gene or member of a gene family from Table I may be compared to the expression levels found in normal subject. The expression level may also be compared to the expression levels observed in sample tissues exhibiting a similar disease state, which may aid in its diagnosis. The comparison of expression data, as well as available sequences or other information may be done by a researcher or diagnostician or may be done with the aid of a computer and databases as described above. Such methods may be used to diagnose or identify conditions characterized by abnormal expression of the genes that are described in Table I.

The methods of the present invention may be particularly useful in diagnosing or monitoring effectiveness of treatment regimen. Compounds that modulate the expression of one or more genes or gene families or proteins or expressions regulators or products of proteins or receptors of proteins identified in Table I and/or II and/or modulate the activity of one or more of the proteins or expressions regulators or products of proteins or receptors of proteins encoded by one or more of the genes or members of a gene family identified in Table I will be useful in diagnosis, monitoring, and evaluation of patient responses to treatment regimen.

### EXAMPLES

### Example A

An in vitro cell line of BEAS-2B cells can be infected with rhinovirus RV-16. The cells are then exposed to various compounds and extracts and subsequently levels of respiratory biomarker proteins can be assayed. Extracts and compounds are identified as regulating the respiratory biomarker proteins by monitoring the levels of the respiratory biomarker proteins after exposure of the infected cells to the extracts and compounds and comparing to the levels of the respiratory biomarker proteins in infected cells that have not been exposed to extracts and compounds.

In the example, the test ingredients are extracts of the herb andrographis paniculata, or its principal component, andrographolide. The test ingredients are tested at a level of 5 μM andrographolide content. The respiratory biomarker protein is IP-10 (CXCL10), a chemotactic agent.

| Test Ingredient | IP-10 |
|---|---|
| (5 μM andrographolide | (CXCL10) |
| content) | pg/ml |
| Control | 61.99 |
| Andrographolide | 18.07 |
| Andrographis A | 0.46 |
| Andrographis B | 2.34 |

Andrographis A is sourced from Sabinsa, Piscataway, NJ.
Andrographis B is sourced from GNC, Pittsburgh, PA.

A substantial reduction in the chemotactic protein level can be seen for the test ingredients compared to the control leg.

### Example B

The effect of test compounds on the course of rhinoviral infections in naturally-induced colds in humans can be assessed by monitoring respiratory protein biomarker levels. Nasal lavage fluid is collected from subjects exhibiting the first signs of a cold. The subjects are then given treatments and nasal lavage samples are taken on the following day.

The treatment consists of andrographis paniculata extract standardized to 20 mg total andrographolides, 28.8 mg eleutherococcus senticosus extract and 650 mg curcumin (turmeric extract). This combination is dosed three times daily. The respiratory biomarker protein is IP-10 (CXCL10), a chemotactic agent. The levels are assayed on the day following treatment with a statistical correction for the baseline values prior to treatment.

| | IP-10 |
|---|---|
| | (CXCL10) |
| Test Ingredient | pg/ml |
| Control | 8183 |
| Andrographis, | 3584 |
| Eleutherococcus, | |
| Curcumin Combination | |

Andrographis paniculata and Eleutherococcus senticosus are available from the Swedish Herbal Institute, Göteborg, Sweden.
Curcumin is available from Sabinsa, Piscataway, NJ.

A substantial reduction in the chemotactic protein level is seen for the test ingredients compared to the control leg.

### Example C

The effect of test compounds on the course of rhinoviral infections in naturally-induced colds in humans can be assessed by monitoring respiratory protein biomarker levels. Nasal lavage fluid is collected from subjects exhibiting the first signs of a cold. The subjects are then given treatments and nasal lavage samples are taken on the following day.

The treatment consists of 400 mg ibuprofen and 4 mg chlorpheniramine maleate. This combination is dosed three times daily. The respiratory biomarker protein is MCP1 (CCL2), a chemotactic agent. The levels are assayed on the day following treatment with a statistical correction for the baseline values prior to treatment.

| | MCP1 (CCL2) |
|---|---|
| Test Ingredient | pg/ml |
| Control | 271 |
| Ibuprofen and | 163 |
| Chlorpheniramine Maleate | |
| Combination. | |

Ibuprofen is available from Wyeth Consumer Healthcare, Wilmington DE.
Chlorpheniramine Maleate is available from Schering Plough, Kenilworth NJ.

A substantial reduction in the chemotactic protein level is seen for the test ingredients compared to the control leg.

### Examples D, E & F

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified concentrations are weight-weight percents, unless otherwise specified.

Turmeric extract may be obtained from Sabinsa Corporation, Piscataway, N.J. Eleutherococcus and Andrographis extracts may be obtained from Dansk Droge, Denmark.

### Example D

| Component | Amount per Capsule | |
|---|---|---|
| Andrographis paniculata extract | 51.0 | mg* |
| Turmeric extract | 166.7 | mg** |
| Eleutherococcus Senticosus extract | 7.2 | mg*** |
| Piperine | 1.2 | mg |
| Microcrystalline Cellulose, Avicel PH 200 | 171.9 | mg |
| Magnesium Stearate | 2.0 | mg |

*51.0 mg Andrographis paniculata containing 5 mg andrographilides.
**166.7 mg Turmeric extract containing 158.3 mg curcuminoids.
***7.2 mg Eleutherococcus senticosus extract, equivalent to 120 mg Eleutherococcus senticosus root.

The andrographis, turmeric, eleutherococcus, piperine and cellulose powders are mixed together. The magnesium stearate is then added and the entire blend is mixed. The resulting powder blend is dispensed into capsules containing 400 mg each. Dosage is four capsules taken three times daily.

### Example E

| Component | Amount per Capsule | |
|---|---|---|
| Andrographis paniculata extract | 102.0 | mg* |
| Turmeric extract | 333.3 | mg** |
| Eleutherococcus senticosus extract | 14.4 | mg*** |
| Piperine | 2.4 | mg |
| Microcrystalline Cellulose, Avicel PH 200 | 144.9 | mg |
| Magnesium Stearate | 3.0 | mg |

*102 mg Andrographis paniculata containing 10 mg andrographilides.
**333.3 mg Turmeric extract containing 316.7 mg curcuminoids.
***14.4 mg Eleutherococcus senticosus extract, equivalent to 240 mg Eleutherococcus senticosus root.

The andrographis, turmeric, eleutherococcus, piperine and cellulose powders are mixed together. The magnesium stearate is then added and the entire blend is mixed. The resulting powder blend is dispensed into capsules containing 600 mg each. Dosage is two capsules taken three times daily.

### Example F

| Component | Amount per Tablet | |
|---|---|---|
| Andrographis paniculata extract | 102.0 | mg* |
| Turmeric extract | 333.3 | mg** |
| Eleutherococcus senticosus extract | 14.4 | mg*** |
| Piperine | 2.4 | mg |
| Povidone | 18.0 | mg |
| Croscarmellose, sodium | 12.0 | mg |
| Microcrystalline Cellulose, Avicel PH 200 | 114.9 | mg |
| Magnesium Stearate | 3.0 | mg |

*102 mg Andrographis paniculata containing 10 mg andrographilides.
**333.3 mg Turmeric extract containing 316.7 mg curcuminoids.
***14.4 mg Eleutherococcus senticosus extract, equivalent to 240 mg Eleutherococcus senticosus root.

The andrographis, turmeric, eleutherococcus, piperine, povidone, cellulose and half the croscarmellose sodium are mixed together with a small amount of water until granulation occurs. The granulation is oven-dried to remove the water, and the blend is milled. The remaining half of the croscarmellose sodium and the magnesium stearate is then added and the entire blend is mixed. The resulting powder blend is compressed into tablets containing 600 mg each. The tablets may be optionally coated with sugar or film coating. Dosage is two capsules taken three times daily.

### Example G

Because multiple chemokines may be upregulated after rhinovirus infection, a method to block chemotaxis is by using broad-spectrum chemokine receptor antagonists. PBMC's are typically a mixture of monocytes and lymphocytes, that is, blood leukocytes from which granulocytes have been separated and removed. PBMC's can be labeled with a fluorescent dye such as Cell Tracker Green, available from Lonza Group Ltd, Basel, Switzerland, and the inhibition of migration in response to a chemokine can be monitored. Chemotactic migration may be induced by SDF1a (Stromal-Derived Factor-1 alpha) available from US Biological, Swampscott, Mass. SDF1a may induce chemotactic migration by binding to a chemotactic receptor such as CXCR4 and others that may occur on the PBMC's. The inhibition of chemotactic migration may be observed upon application of a potential chemotactic inhibitor, such as vMIP-II (viral Macrophage Inflammatory Protein-II) available from Sigma-Aldrich, St. Louis, Mo. vMIP-II can bind to chemotactic receptors such as CCR2, CCR5 and others that may occur on the PBMC's. A chemotactic inhibitor may show partial or complete inhibition of chemotaxis, and may show a dose dependence.

| vMIP-II | % Inhibition of SDF1a- |
|---|---|
| (concentration μg/mL) | induced Chemotaxis |
| 0.01564 | 50% |
| 0.22 | 100% |

### Example H

Test compounds such as ethoxyquin, eugenol or dihydroeugenol, available from Sigma-Aldrich, St. Louis, Mo., can be assayed for inhibition of cyclooxygenase activity using purified enzymes. Test compounds may be assayed for inhibition of prostaglandin production via contacting them individually with cells that have been infected with rhinovirus. An assay for prostaglandin is available from Cisbio International, Bedford Mass. One cell line suitable for infection by rhinovirus is A549 (ATCC designation CCL-185), a human epithelial lung carcinoma available from ATCC, Manassas, Va. The test results may be reported as the IC50 (Inhibitory Concentration 50%), the concentration at which the PGE2 formation or COX-1 or COX-2 activity is at one-half its maximal value. A COX assay is available from Cayman Chemical, Ann Arbor, Mich.

| | IC50 (uM) | | |
|---|---|---|---|
| | PGE2 | COX-1 | COX-2 |
| Compound | formation | activity | activity |
| ETHOXYQUIN | 0.03 | 459 | 54 |
| EUGENOL | 0.42 | 42 | 15 |
| DIHYDROEUGENOL | 0.38 | 82 | 75 |

### Example I

Test compounds such as curcumin (available from Sigma-Aldrich, St. Louis Mo.) and Ro1069920 (available from CalBiochem, EMD Biosciences, Darmstadt Germany) can be assayed for inhibition of NF-kB activity by measuring the decrease in translocation of NF-kB using the NF-kB Activation HitKit® HCS Reagent Kit (available from Cellomics, Pittsburgh, Pa.). Test compounds may be assayed for inhibition of NF-kB translocation via contacting them individually with cells that have been infected with rhinovirus or activated using IL1β. Two cell lines suitable for infection by rhinovirus are A549 (a human epithelial lung carcinoma, ATCC CCL-185), and BEAS-2B (human bronchial epithelial cell line, ATCC CRL-9609). In this example, both cell types were pre-treated with IL1b (0.05 ng/ml for A549 cells and 0.5 ng/ml for BEAS-2B cells) for 30 min to stimulate the NF-kB translocation to the nucleus before addition of test inhibitors. After test inhibitor addition, the cells were further incubated for another 30 min. Cells were fixed and assayed using the Cellomics NFKB Activation HitKit® HCS Reagent Kit. The test results may be reported as the IC50 (Inhibitory Concentration 50%), the concentration at which the translocation of NF-□B is at one-half its maximal value.

IC50 (uM) of
IL1b-induced NFkB
nuclear translocation

| Compound | A549 | BEAS-2B |
|---|---|---|
| Ro1069920 | 6.7 | 0.8 |
| Curcumin | 37.5 | 5.9 |

### Example J

Components from an extract of green tea (camellia sinensis) such as epigallocatechin and epigallocatecfhine gallate may be placed in proximity with ICAM-1 (human rhinovirus receptor encoded by a gene of Table I). The extent of binding of the components on expression of ICAM-1 may be determined by a standard competitive binding assay. Those components that substantially bind ICAM-1 may be identified as compounds involved in regulating rhinovirus infection by inhibition through effects on viral binding and uptake.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited herein are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

TABLE I
List of Top Genes Expressed in 48 hr Nasal Samples from Subjects Infected with RV16

| Transcript_ID | Title | Acronym |
|---|---|---|
| 202869_at | 2′,5′-oligoadenylate synthetase 1, 40/46 kDa | OAS1 |
| 205552_s_at | 2′,5′-oligoadenylate synthetase 1, 40/46 kDa | OAS1 |
| 204972_at | 2′-5′-oligoadenylate synthetase 2, 69/71 kDa | OAS2 |
| 206553_at | 2′-5′-oligoadenylate synthetase 2, 69/71 kDa | OAS2 |
| 228607_at | 2′-5′-oligoadenylate synthetase 2, 69/71 kDa | OAS2 |
| 218400_at | 2′-5′-oligoadenylate synthetase 3, 100 kDa | OAS3 |
| 232666_at | 2′-5′-oligoadenylate synthetase 3, 100 kDa | OAS3 |
| 205660_at | 2′-5′-oligoadenylate synthetase-like | OASL |
| 210797_s_at | 2′-5′-oligoadenylate synthetase-like | OASL |
| 219684_at | 28 kD interferon responsive protein | IFRG28 |
| 204607_at | 3-hydroxy-3-methylglutaryl-Coenzyme A synthase 2 | HMGCS2 |
| | (mitochondrial) | |
| 1555785_a_at | 5′-3′exoribonuclease 1 | XRN1 |
| 233632_s_at | 5′-3′exoribonuclease 1 | XRN1 |
| 223298_s_at | 5′-nucleotidase, cytosolic III | NT5C3 |
| 222162_s_at | a disintegrin-like and metalloprotease (reprolysin type) with | ADAMTS1 |
| | thrombospondin type 1 motif, 1 | |
| 237281_at | A kinase (PRKA) anchor protein 14 | AKAP14 |
| 237282_s_at | A kinase (PRKA) anchor protein 14 | AKAP14 |
| 206513_at | absent in melanoma 2 | AIM2 |
| 1557418_at | Acyl-CoA synthetase long-chain family member 4 | ACSL4 |
| 201786_s_at | adenosine deaminase, RNA-specific | ADAR |
| 213217_at | adenylate cyclase 2 (brain) | ADCY2 |
| 225342_at | Adenylate kinase 3 | AK3L1 |
| 1553734_at | adenylate kinase 7 | AK7 |
| 209869_at | adrenergic, alpha-2A-, receptor | ADRA2A |
| 206170_at | adrenergic, beta-2-, receptor, surface | ADRB2 |
| 202912_at | adrenomedullin | ADM |
| 206262_at | alcohol dehydrogenase 1C (class I), gamma polypeptide | ADH1C |
| 207544_s_at | alcohol dehydrogenase 6 (class V) | ADH6 |
| 214261_s_at | alcohol dehydrogenase 6 (class V) | ADH6 |
| 210505_at | alcohol dehydrogenase 7 (class IV), mu or sigma polypeptide | ADH7 |
| 205640_at | aldehyde dehydrogenase 3 family, member B1 | ALDH3B1 |
| 211004_s_at | aldehyde dehydrogenase 3 family, member B1 | ALDH3B1 |
| 203609_s_at | aldehyde dehydrogenase 5 family, member A1 (succinate- | ALDH5A1 |
| | semialdehyde dehydrogenase) | |
| 209901_x_at | allograft inflammatory factor 1 | AIF1 |
| 213095_x_at | allograft inflammatory factor 1 | AIF1 |
| 215051_x_at | allograft inflammatory factor 1 | AIF1 |
| 1552698_at | alpha tubulin-like | MGC16703 |
| 205156_s_at | amiloride-sensitive cation channel 2, neuronal | ACCN2 |
| 1555284_at | amyotrophic lateral sclerosis 2 (juvenile) | ALS2 |
| 211110_s_at | androgen receptor (dihydrotestosterone receptor; testicular | AR |
| | feminization; spinal and bulbar muscular atrophy; Kennedy | |
| | disease) | |
| 219962_at | angiotensin I converting enzyme (peptidyl-dipeptidase A) 2 | ACE2 |
| 210486_at | ankyrin repeat and MYND domain containing 1 | ANKMY1 |
| 238439_at | ankyrin repeat domain 22 | ANKRD22 |
| 239196_at | ankyrin repeat domain 22 | ANKRD22 |
| 211712_s_at | annexin A9 | ANXA9 |
| 210873_x_at | apolipoprotein B mRNA editing enzyme, catalytic | APOBEC3A |
| | polypeptide-like 3A | |
| 209546_s_at | apolipoprotein L, 1 | APOL1 |
| 221653_x_at | apolipoprotein L, 2 | APOL2 |
| 221087_s_at | apolipoprotein L, 3 | APOL3 |
| 1555600_s_at | apolipoprotein L, 4 | APOLA |
| 223801_s_at | apolipoprotein L, 4 | APOL4 |
| 219716_at | apolipoprotein L, 6 | APOL6 |
| 241869_at | apolipoprotein L, 6 | APOL6 |
| 221241_s_at | apoptosis regulator BCL-G | BCLG |
| 204446_s_at | arachidonate 5-lipoxygenase | ALOX5 |
| 214366_s_at | arachidonate 5-lipoxygenase | ALOX5 |
| 204445_s_at | arachidonate 5-lipoxygenase | ALOX5 |
| 213952_s_at | arachidonate 5-lipoxygenase | ALOX5 |
| 238878_at | aristaless related homeobox | ARX |
| 223794_at | armadillo repeat containing 4 | ARMC4 |
| 205969_at | arylacetamide deacetylase (esterase) | AADAC |
| 215407_s_at | astrotactin 2 | ASTN2 |
| 213138_at | AT rich interactive domain 5A (MRF1-like) | ARID5A |
| 232265_at | ataxin 7-like 1 | ATXN7L1 |
| 237400_at | ATP synthase, H+ transporting, mitochondrial F0 complex, | ATP5S |
| | subunit s (factor B) | |
| 239859_x_at | ATP synthase, H+ transporting, mitochondrial F0 complex, | ATP5S |
| | subunits (factor B) | ATP5S |
| 214255_at | ATPase, Class V, type 10A | ATP10A |
| 207583_at | ATP-binding cassette, sub-family D (ALD), member 2 | ABCD2 |
| 206076_at | B7 gene | B7 |
| 205662_at | B9 protein | EPPB9 |
| 210534_s_at | B9 protein | EPPB9 |
| 210538_s_at | baculoviral IAP repeat-containing 3 | BIRC3 |
| 221530_s_at | basic helix-loop-helix domain containing, class B, 3 | BHLHB3 |
| 220766_at | B-cell translocation gene 4 | BTG4 |
| 203728_at | BCL2-antagonist/killer 1 | BAK1 |
| 221241_s_at | BCL2-like 14 (apoptosis facilitator) | BCL2L14 |
| 220087_at | beta-carotene 15,15′-monooxygenase 1 | BCMO1 |
| 201641_at | bone marrow stromal cell antigen 2 | BST2 |
| 218876_at | brain specific protein | CGI-38 |
| 206382_s_at | brain-derived neurotrophic factor | BDNF |
| 202946_s_at | BTB (POZ) domain containing 3 | BTBD3 |
| 1554712_a_at | BXMAS2-10 | BXMAS2-10 |
| 233662_at | cadherin-like 26 | CDH26 |
| 209530_at | calcium channel, voltage-dependent, beta 3 subunit | CACNB3 |
| 214475_x_at | calpain 3, (p94) | CAPN3 |
| 208063_s_at | calpain 9 | CAPN9 |
| 210641_at | calpain 9 | CAPN9 |
| 229228_at | cAMP responsive element binding protein 5 | CREB5 |
| 220168_at | cancer susceptibility candidate 1 | CASC1 |
| 234732_s_at | CAP-binding protein complex interacting protein 1 | FLJ23588 |
| 219634_at | carbohydrate (chondroitin 4) sulfotransferase 11 | CHST11 |
| 223737_x_at | carbohydrate (N-acetylgalactosamine 4-0) sulfotransferase 9 | CHST9 |
| 224400_s_at | carbohydrate (N-acetylgalactosamine 4-0) sulfotransferase 9 | CHST9 |
| 219182_at | carbohydrate (N-acetylglucosamine 6-O) sulfotransferase 5 | CHST5 |
| 205379_at | carbonyl reductase 3 | CBR3 |
| 209616_s_at | carboxylesterase 1 (monocyte/macrophage serine esterase 1) | CES1 |
| 206576_s_at | carcinoembryonic antigen-related cell adhesion molecule 1 | CEACAM1 |
| | (biliary glycoprotein) | |
| 209498_at | carcinoembryonic antigen-related cell adhesion molecule 1 | CEACAM1 |
| | (biliary glycoprotein) | |
| 211883_x_at | carcinoembryonic antigen-related cell adhesion molecule 1 | CEACAM1 |
| | (biliary glycoprotein) | |
| 211889_x_at | carcinoembryonic antigen-related cell adhesion molecule 1 | CEACAM1 |
| | (biliary glycoprotein) | |
| 210563_x_at | CASP8 and FADD-like apoptosis regulator | CFLAR |
| 209939_x_at | CASP8 and FADD-like apoptosis regulator | CELAR |
| 211862_x_at | CASP8 and FADD-like apoptosis regulator | CFLAR |
| 211317_s_at | CASP8 and FADD-like apoptosis regulator | CFLAR |
| 239629_at | CASP8 and FADD-like apoptosis regulator | CFLAR |
| 210564_x_at | CASP8 and FADD-like apoptosis regulator | CFLAR |
| 209508_x_at | CASP8 and FADD-like apoptosis regulator | CFLAR |
| 211367_s_at | caspase 1, apoptosis-related cysteine protease (interleukin 1, | CASP1 |
| | beta, convertase) | |
| 211368_s_at | caspase 1, apoptosis-related cysteine protease (interleukin 1, | CASP1 |
| | beta, convertase) | |
| 205467_at | caspase 10, apoptosis-related cysteine protease | CASP10 |
| 213596_at | caspase 4, apoptosis-related cysteine protease | CASP4 |
| 209310_s_at | caspase 4, apoptosis-related cysteine protease | CASP4 |
| 207500_at | caspase 5, apoptosis-related cysteine protease | CASP5 |
| 207181_s_at | caspase 7, apoptosis-related cysteine protease | CASP7 |
| 207686_s_at | caspase 8, apoptosis-related cysteine protease | CASP8 |
| 201432_at | catalase | CAT |
| 238363_at | catalase | CAT |
| 1565633_at | Catenin (cadherin-associated protein), delta 1 | CTNND1 |
| 203645_s_at | CD163 antigen | CD163 |
| 215049_x_at | CD163 antigen | CD163 |
| 223834_at | CD274 antigen | CD274 |
| 227458_at | CD274 antigen | CD274 |
| 228766_at | CD36 antigen (collagen type I receptor, thrombospondin | CD36 |
| | receptor) | |
| 211075_s_at | CD47 antigen (Rh-related antigen, integrin-associated signal | CD47 |
| | transducer) | |
| 213857_s_at | CD47 antigen (Rh-related antigen, integrin-associated signal | CD47 |
| | transducer) | |
| 203507_at | CD68 antigen | CD68 |
| 209795_at | CD69 antigen (p60, early T-cell activation antigen) | CD69 |
| 237009_at | CD69 antigen (p60, early T-cell activation antigen) | CD69 |
| 214049_x_at | CD7 antigen (p41) | CD7 |
| 1554519_at | CD80 antigen (CD28 antigen ligand 1, B7-1 antigen) | CD80 |
| 204440_at | CD83 antigen (activated B lymphocytes, immunoglobulin | CD83 |
| | superfamily) | |
| 211192_s_at | CD84 antigen (leukocyte antigen) | CD84 |
| 210895_s_at | CD86 antigen (CD28 antigen ligand 2, B7-2 antigen) | CD86 |
| 205288_at | CDC14 cell division cycle 14 homolog A (S. cerevisiae) | CDC14A |
| 210742_at | CDC14 cell division cycle 14 homolog A (S. cerevisiae) | CDC14A |
| 240735_at | CDC42 binding protein kinase alpha (DMPK-like) | CDC42BPA |
| 204693_at | CDC42 effector protein (Rho GTPase binding) 1 | CDC42EP1 |
| 1569004_at | CDNA clone IMAGE: 30349460, partial cds | |
| 1570165_at | CDNA clone IMAGE: 3895112, containing frame-shift errors | |
| 1559817_at | CDNA clone IMAGE: 3961179, partial cds | |
| 231046_at | CDNA clone IMAGE: 4329532, partial cds | |
| 238910_at | CDNA clone IMAGE: 4779711, partial cds | |
| 1559103_s_at | CDNA clone IMAGE: 4791593, partial cds | |
| 228108_at | CDNA clone IMAGE: 5263177, partial cds | |
| 235532_at | CDNA clone IMAGE: 5302913, partial cds | |
| 236522_at | CDNA FLJ25684 fis, clone TST04185 | |
| 213429_at | CDNA FLJ26539 fis, clone KDN09310 | |
| 225996_at | CDNA FLJ36725 fis, clone UTERU2012230 | |
| 1557383_a_at | CDNA FLJ38112 fis, clone D3OST2002272 | |
| 1556938_a_at | CDNA FLJ38433 fis, clone FEBRA2014578 | |
| 227061_at | CDNA FLJ44429 fis, clone UTERU2015653 | |
| 229190_at | CDNA FLJ90295 fis, clone NT2RP2000240. | |
| 214567_s_at | chemokine (C motif) ligand 1 | XCL1 |
| 206366_x_at | chemokine (C motif) ligand 2 | XCL2 |
| 32128_at | chemokine (C-C motif) ligand 18 (pulmonary and activation- | CCCL18 |
| | regulated) | |
| 210072_at | chemokine (C-C motif) ligand 19 | CCL19 |
| 216598_s_at | chemokine (C-C motif) ligand 2 | CCL2 |
| 205476_at | chemokine (C-C motif) ligand 20 | CCL20 |
| 205114_s_at | chemokine (C-C motif) ligand 3 | CCL3L1 |
| 1405_i_at | chemokine (C-C motif) ligand 5 | CCL5 |
| 1555759_a_at | chemokine (C-C motif) ligand 5 | CCL5 |
| 204655_at | chemokine (C-C motif) ligand 5 | CCL5 |
| 214038_at | chemokine (C-C motif) ligand 8 | CCL8 |
| 205098_at | chemokine (C-C motif) receptor 1 | CCR1 |
| 205099_s_at | chemokine (C-C motif) receptor 1 | CCR1 |
| 206991_s_at | chemokine (C-C motif) receptor 5 | CCR5 |
| 211434_s_at | chemokine (C-C motif) receptor-like 2 | CCRL2 |
| 203687_at | chemokine (C—X3—C motif) ligand 1 | CX3CL1 |
| 823_at | chemokine (C—X3—C motif) ligand 1 | CX3CL1 |
| 204470_at | chemokine (C—X—C motif) ligand 1 (melanoma growth | CXCL1 |
| | stimulating activity, alpha) | |
| 204533_at | chemokine (C—X—C motif) ligand 10 | CXCL10 |
| 210163_at | chemokine (C—X—C motif) ligand 11 | CXCL11 |
| 211122_s_at | chemokine (C—X—C motif) ligand 11 | CXCL11 |
| 205242_at | chemokine (C—X—C motif) ligand 13 (B-cell chemoattractant) | CXCL13 |
| 222484_s_at | chemokine (C—X—C motif) ligand 14 | CXCL14 |
| 209774_x_at | chemokine (C—X—C motif) ligand 2 | CXCL2 |
| 207850_at | chemokine (C—X—C motif) ligand 3 | CXCL3 |
| 215101_s_at | chemokine (C—X—C motif) ligand 5 | CXCL5 |
| 203915_at | chemokine (C—X—C motif) ligand 9 | CXCL9 |
| 1555705_a_at | chemokine-like factor super family 3 | CKLFSF3 |
| 224998_at | chemokine-like factor super family 4 | CKLFS4 |
| 225009_at | chemokine-like factor super family 4 | CKLFS4 |
| 209395_at | chitinase 3-like 1 (cartilage glycoprotein-39) | CHI3L1 |
| 209396_s_at | chitinase 3-like 1 (cartilage glycoprotein-39) | CHI3L1 |
| 213415_at | chloride intracellular channel 2 | CLIC2 |
| 221881_s_at | chloride intracellular channel 4 | CLIC4 |
| 206869_at | chondroadherin | CHAD |
| 211571_s_at | chondroitin sulfate proteoglycan 2 (versican) | CSPG2 |
| 207571_x_at | chromosome 1 open reading frame 38 | C1ORF38 |
| 238453_at | chromosome 10 open reading frame 13 | C10ORF13 |
| 241902_at | chromosome 10 open reading frame 48 | C10ORF48 |
| 220344_at | chromosome 11 open reading frame 16 | C11ORF16 |
| 215692_s_at | chromosome 11 open reading frame 8 | C11ORF8 |
| 237974_at | chromosome 14 open reading frame 29 | C14ORF29 |
| 1552950_at | chromosome 15 open reading frame 26 | C15ORF26 |
| 230811_at | chromosome 16 open reading frame 55 | C16ORF55 |
| 225929_s_at | chromosome 17 open reading frame 27 | C17ORF27 |
| 225931_s_at | chromosome 17 open reading frame 27 | C17ORF27 |
| 230000_at | chromosome 17 open reading frame 27 | C17ORF27 |
| 229542_at | chromosome 20 open reading frame 85 | C20ORF85 |
| 1558333_at | chromosome 22 open reading frame 15 | C22ORF15 |
| 1558334_a_at | chromosome 22 open reading frame 15 | C22ORF15 |
| 240232_at | Chromosome 3 open reading frame 1 | C3ORF1 |
| 229152_at | chromosome 4 open reading frame 7 | C4ORF7 |
| 220751_s_at | chromosome 5 open reading frame 4 | C5ORF4 |
| 1559051_s_at | chromosome 6 open reading frame 150 | C6ORF150 |
| 233438_at | Chromosome 6 open reading frame 162 | C6ORF162 |
| 230695_s_at | chromosome 6 open reading frame 206 | C6ORF206 |
| 231070_at | chromosome 6 open reading frame 71 | C6ORF71 |
| 226603_at | chromosome 7 open reading frame 6 | SAMD9L |
| 230036_at | chromosome 7 open reading frame 6 | SAMD9L |
| 235643_at | chromosome 7 open reading frame 6 | SAMD9L |
| 243271_at | Chromosome 7 open reading frame 6 | SAMD9L |
| 218541_s_at | chromosome 8 open reading frame 4 | C8ORF4 |
| 221946_at | chromosome 9 open reading frame 116 | C9ORF116 |
| 59437_at | chromosome 9 open reading frame 116 | C9ORF116 |
| 1557014_a_at | chromosome 9 open reading frame 122 | C9ORF122 |
| 229976_at | chromosome 9 open reading frame 18 | C9ORF18 |
| 229012_at | chromosome 9 open reading frame 24 | C9ORF24 |
| 1553433_at | chromosome 9 open reading frame 93 | C9ORF93 |
| 1553905_at | chromosome X open reading frame 22 | CXORF22 |
| 231389_at | chromosome X open reading frame 41 | CXORF41 |
| 228739_at | Cilia-associated protein (CYS1) | |
| 218223_s_at | CK2 interacting protein 1; HQ0024c protein | CKIP-1 |
| 218182_s_at | claudin 1 | CLDN1 |
| 222549_at | claudin 1 | CLDN1 |
| 1556687_a_at | claudin 10 | CLDN10 |
| 214598_at | claudin 8 | CLDN8 |
| 243585_at | Clone DNA57844 ELIP488 (UNQ488) mRNA, complete cds | |
| 209716_at | colony stimulating factor 1 (macrophage) | CSF1 |
| 207442_at | colony stimulating factor 3 (granulocyte) | CSF3 |
| 1555229_a_at | complement component 1, s subcomponent | C1S |
| 208747_s_at | complement component 1, s subcomponent | C1S |
| 209906_at | complement component 3a receptor 1 | C3AR1 |
| 208451_s_at | complement component 4A | C4B |
| 227209_at | contactin 1 | CNTN1 |
| 229831_at | contactin 3 (plasmacytoma associated) | CNTN3 |
| 244632_at | contactin 5 | CNTN5 |
| 1556209_at | C-type lectin domain family 2, member B | CLEC2B |
| 209732_at | C-type lectin domain family 2, member B | CLEC2B |
| 1555214_a_at | C-type lectin domain family 7, member A | CLEC7A |
| 202284_s_at | cyclin-dependent kinase inhibitor 1A (p21, Cip1) | CDKN1A |
| 210140_at | cystatin F (leukocystatin) | CST7 |
| 205081_at | cysteine-rich protein 1 (intestinal) | CRIP1 |
| 230866_at | cysteinyl leukotriene receptor 1 | CYSLTR1 |
| 231747_at | cysteinyl leukotriene receptor 1 | CYSLTR1 |
| 203922_s_at | cytochrome b-245, beta polypeptide (chronic granulomatous | CYBB |
| | disease) | |
| 203923_s_at | cytochrome b-245, beta polypeptide (chronic granulomatous | CYBB |
| | disease) | |
| 1553434_at | cytochrome P450 4Z2 pseudogene | CYP4Z2P |
| 206504_at | cytochrome P450, family 24, subfamily A, polypeptide 1 | CYP24A1 |
| 206424_at | cytochrome P450, family 26, subfamily A, polypeptide 1 | CYP26A1 |
| 220432_s_at | cytochrome P450, family 39, subfamily A, polypeptide 1 | CYP39A1 |
| 223961_s_at | cytokine inducible SH2-containing protein | CISH |
| 231794_at | cytotoxic T-lymphocyte-associated protein 4 | CTLA4 |
| 236341_at | cytotoxic T-lymphocyte-associated protein 4 | CTLA4 |
| 218943_s_at | DEAD (Asp-Glu-Ala-Asp) box polypeptide 58 | DDX58 |
| 222793_at | DEAD (Asp-Glu-Ala-Asp) box polypeptide 58 | DDX58 |
| 242961_x_at | DEAD (Asp-Glu-Ala-Asp) box polypeptide 58 | DDX58 |
| 213420_at | DEAH (Asp-Glu-Ala-Asp/His) box polypeptide 57 | DHX57 |
| 210397_at | defensin, beta 1 | DEFB1 |
| 225415_at | deltex 3-like (Drosophila) | DTX3L |
| 231552_at | DKFZP434C212 protein | GAPVD1 |
| 202887_s_at | DNA-damage-inducible transcript 4 | DDIT4 |
| 1560020_at | DnaJ (Hsp40) homolog, subfamily C, member 13 | DNAJC13 |
| 207311_at | double C2-like domains, beta | DOC2B |
| 1552708_a_at | dual specificity phosphatase 19 | DUSP19 |
| 204794_at | dual specificity phosphatase 2 | DUSP2 |
| 209457_at | dual specificity phosphatase 5 | DUSP5 |
| 208891_at | dual specificity phosphatase 6 | DUSP6 |
| 208892_s_at | dual specificity phosphatase 6 | DUSP6 |
| 208893_s_at | dual specificity phosphatase 6 | DUSP6 |
| 1565337_at | dynein, axonemal, heavy polypeptide 6 | DNAH6 |
| 240857_at | dynein, axonemal, heavy polypeptide 9 | DNAH9 |
| 220636_at | dynein, axonemal, intermediate polypeptide 2 | DNAI2 |
| 227081_at | dynein, axonemal, light intermediate polypeptide 1 | DNALI1 |
| 1565149_at | dynein, cytoplasmic, heavy polypeptide 2 | DNCH2 |
| 235273_at | dyslexia susceptibility 1 candidate 1 | DYX1C1 |
| 1562921_at | E1A binding protein p300 | EP300 |
| 220624_s_at | E74-like factor 5 (ets domain transcription factor) | ELF5 |
| 205249_at | early growth response 2 (Krox-20 homolog, Drosophila) | EGR2 |
| 209392_at | ectonucleotide pyrophosphatase/phosphodiesterase 2 | ENPP2 |
| | (autotaxin) | |
| 210839_s_at | ectonucleotide pyrophosphatase/phosphodiesterase 2 | ENPP2 |
| | (autotaxin) | |
| 201842_s_at | EGF-containing fibulin-like extracellular matrix protein 1 | EFEMP1 |
| 207111_at | egf-like module containing, mucin-like, hormone receptor- | EMR1 |
| | like 1 | |
| 207610_s_at | egf-like module containing, mucin-like, hormone receptor- | EMR2 |
| | like 2 | |
| 244660_at | ELAV (embryonic lethal, abnormal vision, Drosophila)-like 1 | ELAVL1 |
| | (Hu antigen R) | |
| 227180_at | ELOVL family member 7, elongation of long chain fatty | ELOVL7 |
| | acids (yeast) | |
| 204858_s_at | endothelial cell growth factor 1 (platelet-derived) | ECGF1 |
| 217497_at | endothelial cell growth factor 1 (platelet-derived) | ECGF1 |
| 206758_at | endothelin 2 | EDN2 |
| 204464_s_at | endothelin receptor type A | EDNRA |
| 203249_at | enhancer of zeste homolog 1 (Drosophila) | EZH1 |
| 201313_at | enolase 2 (gamma, neuronal) | ENO2 |
| 227609_at | epithelial stromal interaction 1 (breast) | EPSTI1 |
| 239979_at | Epithelial stromal interaction 1 (breast) | EPSTI1 |
| 235276_at | epithelial stromal interaction 1 (breast) | EPSTI1 |
| 222646_s_at | ERO1-like (S. cerevisiae) | ERO1L |
| 206710_s_at | erythrocyte membrane protein band 4.1-like 3 | EPB41L3 |
| 212681_at | erythrocyte membrane protein band 4.1-like 3 | EPB41L3 |
| 221680_s_at | ets variant gene 7 (TEL2 oncogene) | ETV7 |
| 224225_s_at | ets variant gene 7 (TEL2 oncogene) | ETV7 |
| 204211_x_at | eukaryotic translation initiation factor 2-alpha kinase 2 | EIF2AK2 |
| 223533_at | factor for adipocyte differentiation 158 | LRRC8C |
| 1554547_at | family with sequence similarity 13, member C1 | FAM13C1 |
| 226804_at | family with sequence similarity 20, member A | FAM20A |
| 241981_at | family with sequence similarity 20, member A | FAM20A |
| 242945_at | family with sequence similarity 20, member A | FAM20A |
| 243221_at | Family with sequence similarity 20, member A | FAM20A |
| 244457_at | Family with sequence similarity 20, member C | FAM20C |
| 221766_s_at | family with sequence similarity 46, member A | FAM46A |
| 224973_at | family with sequence similarity 46, member A | FAM46A |
| 204780_s_at | Fas (TNF receptor superfamily, member 6) | FAS |
| 204781_s_at | Fas (TNF receptor superfamily, member 6) | FAS |
| 215719_x_at | Fas (TNF receptor superfamily, member 6) | FAS |
| 216252_x_at | Fas (TNF receptor superfamily, member 6) | FAS |
| 210865_at | Fas ligand (TNF superfamily, member 6) | FASLG |
| 1554899_s_at | Fc fragment of IgE, high affinity I, receptor for; gamma | FCER1G |
| | polypeptide | |
| 204232_at | Fc fragment of IgE, high affinity I, receptor for; gamma | FCER1G |
| | polypeptide | |
| 216950_s_at | Fc fragment of IgG, high affinity Ia, receptor (CD64) | FCGR1A |
| 203561_at | Fc fragment of IgG, low affinity IIa, receptor (CD32) | FCGR2A |
| 210889_s_at | Fc fragment of IgG, low affinity IIb, receptor (CD32) | FCGR2B |
| 211395_x_at | Fc fragment of IgG, low affinity IIc, receptor for (CD32) | FCGR2C |
| 230645_at | FERM domain containing 3 | FRMD3 |
| 235846_at | Fibrinogen silencer binding protein | RAD54B |
| 222693_at | fibronectin type III domain containing 3B | FNDC3B |
| 205237_at | ficolin (collagen/fibrinogen domain containing) 1 | FCN1 |
| 1570515_a_at | filamin A interacting protein 1 | FILIP1 |
| 215300_s_at | flavin containing monooxygenase 5 | FMO5 |
| 231985_at | flavoprotein oxidoreductase MICAL3 | mical3 |
| 239697_x_at | FLJ42117 protein | FLJ42117 |
| 230757_at | FLJ44796 protein | FLJ44796 |
| 230956_at | FLJ45803 protein | FLJ45803 |
| 1568606_at | FLJ46266 protein | FLJ46266 |
| 226847_at | follistatin | FST |
| 204420_at | FOS-like antigen 1 | FOSL1 |
| 230741_at | Full length insert cDNA clone YX74D05 | |
| 1556190_s_at | Full length insert cDNA clone ZC64C06 | |
| 237690_at | G protein-coupled receptor 115 | GPR115 |
| 210473_s_at | G protein-coupled receptor 125 | GPR125 |
| 209631_s_at | G protein-coupled receptor 37 (endothelin receptor type B- | GPR37 |
| | like) | |
| 223767_at | G protein-coupled receptor 84 | GPR84 |
| 223278_at | gap junction protein, beta 2, 26 kDa (connexin 26) | GJB2 |
| 213685_at | Gene from PAC 886K2, chromosome 1 | |
| 222102_at | glutathione S-transferase A3 | GSTA3 |
| 204550_x_at | glutathione S-transferase M1 | GSTM1 |
| 204418_x_at | glutathione S-transferase M2 (muscle) | GSTM2 |
| 204149_s_at | glutathione S-transferase M4 | GSTM4 |
| 227163_at | glutathione S-transferase omega 2 | GSTO2 |
| 203815_at | glutathione S-transferase theta 1 | GSTT1 |
| 205164_at | glycine C-acetyltransferase (2-amino-3-ketobutyrate | GCAT |
| | coenzyme A ligase) | |
| 205495_s_at | granulysin | GNLY |
| 205488_at | granzyme A (granzyme 1, cytotoxic T-lymphocyte-associated | GZMA |
| | serine esterase 3) | |
| 210164_at | granzyme B (granzyme 2, cytotoxic T-lymphocyte-associated | GZMB |
| | serine esterase 1) | |
| 210321_at | granzyme H (cathepsin G-like 2, protein h-CCPX) | GZMH |
| 204224_s_at | GTP cyclohydrolase 1 (dopa-responsive dystonia) | GCH1 |
| 219243_at | GTPase, IMAP family member 4 | GIMAP4 |
| 219777_at | GTPase, IMAP family member 6 | GIMAP6 |
| 204115_at | guanine nucleotide binding protein (G protein), gamma 11 | GNG11 |
| 204187_at | guanosine monophosphate reductase | GMPR |
| 202269_x_at | guanylate binding protein 1, interferon-inducible, 67 kDa | GBP1 |
| 202270_at | guanylate binding protein 1, interferon-inducible, 67 kDa | GBP1 |
| 231577_s_at | guanylate binding protein 1, interferon-inducible, 67 kDa | GBP1 |
| 231578_at | guanylate binding protein 1, interferon-inducible, 67 kDa | GBP1 |
| 202748_at | guanylate binding protein 2, interferon-inducible | GBP2 |
| 242907_at | guanylate binding protein 2, interferon-inducible | GBP2 |
| 223434_at | guanylate binding protein 3 | GBP3 |
| 235175_at | guanylate binding protein 4 | GBP4 |
| 235574_at | guanylate binding protein 4 | GBP4 |
| 229625_at | Guanylate binding protein 5 | GBP5 |
| 238581_at | Guanylate binding protein 5 | GBP5 |
| 218839_at | hairy/enhancer-of-split related with YRPW motif 1 | HEY1 |
| 44783_s_at | hairy/enhancer-of-split related with YRPW motif 1 | HEY1 |
| 219863_at | hect domain and RLD 5 | HERC5 |
| 219352_at | hect domain and RLD 6 | HERC6 |
| 213069_at | HEG homolog 1 (zebrafish) | HEG1 |
| 1552787_at | helicase (DNA) B | HELB |
| 1552788_a_at | helicase (DNA) B | HELB |
| 1552623_at | hematopoietic SH2 domain containing | HSH2D |
| 203821_at | heparin-binding EGF-like growth factor | HBEGF |
| 206149_at | hepatocellular carcinoma antigen gene 520 | LOC63928 |
| 220812_s_at | HERV-H LTR-associating 2 | HHLA2 |
| 211267_at | homeo box (expressed in ES cells) 1 | HESX1 |
| 238704_at | Homo sapiens, clone IMAGE: 3866695, mRNA | |
| 238887_at | Homo sapiens, clone IMAGE: 3901628, mRNA | |
| 1570298_at | Homo sapiens, clone IMAGE: 4042783, mRNA | |
| 1559777_at | Homo sapiens, clone IMAGE: 4133286, mRNA | |
| 1569675_at | Homo sapiens, clone IMAGE: 4694422, mRNA | |
| 1557118_a_at | Homo sapiens, clone IMAGE: 4812643, mRNA, partial cds | |
| 1558605_at | Homo sapiens, clone IMAGE: 4819775, mRNA | |
| 239343_at | Homo sapiens, clone IMAGE: 4821804, mRNA, partial cds | |
| 240888_at | Homo sapiens, clone IMAGE: 4838406, mRNA | |
| 1561368_at | Homo sapiens, clone IMAGE: 5194369, mRNA | |
| 229072_at | Homo sapiens, clone IMAGE: 5259272, mRNA | |
| 228740_at | Homo sapiens, clone IMAGE: 5276765, mRNA | |
| 227917_at | Homo sapiens, clone IMAGE: 5285282, mRNA | |
| 1561045_a_at | Homo sapiens, clone IMAGE: 5548255, mRNA | |
| 1559534_at | Homo sapiens, clone IMAGE: 5743779, mRNA | |
| 1559535_s_at | Homo sapiens, clone IMAGE: 5743779, mRNA | |
| 232790_at | Homo sapiens, clone IMAGE: 6058191, mRNA | |
| 1561355_at | Homo sapiens, Similar to carnitine deficiency-associated gene | |
| | expressed in ventricle 1, clone IMAGE: 4837775, mRNA | |
| 205221_at | homogentisate 1,2-dioxygenase (homogentisate oxidase) | HGD |
| 219865_at | HSPC157 protein | HSPC157 |
| 227262_at | hyaluronan and proteoglycan link protein 3 | HAPLN3 |
| 230372_at | hyaluronan synthase 2 | HAS2 |
| 223541_at | hyaluronan synthase 3 | HAS3 |
| 242733_at | Hydroxyprostaglandin dehydrogenase 15-(NAD) | HPGD |
| 205404_at | hydroxysteroid (11-beta) dehydrogenase 1 | HSD11B1 |
| 204130_at | hydroxysteroid (11-beta) dehydrogenase 2 | HSD11B2 |
| 244395_at | hypothetical gene supported by AK123449; BX641014 | LOC401155 |
| 244761_at | Hypothetical gene supported by AK126569 | FLJ44606 |
| 229291_at | Hypothetical gene supported by BC053344 | LOC440600 |
| 236909_at | hypothetical LOC129881 | LOC129881 |
| 229930_at | hypothetical LOC150371 | LOC150371 |
| 235606_at | hypothetical LOC344595 | LOC344595 |
| 229107_at | hypothetical LOC388227 | LOC388227 |
| 236674_at | hypothetical LOC388780 | LOC388780 |
| 1557647_a_at | Hypothetical LOC400125 | LOC400125 |
| 222347_at | Hypothetical LOC401131 | LOC401131 |
| 222089_s_at | hypothetical protein AF447587 | LOC146562 |
| 1552639_s_at | hypothetical protein BC009980 | MGC16635 |
| 236285_at | Hypothetical protein BC009980 | MGC16635 |
| 228439_at | hypothetical protein BC012330 | MGC20410 |
| 1552269_at | hypothetical protein BC014608 | LOC128153 |
| 227966_s_at | hypothetical protein BC016861 | LOC90557 |
| 233326_at | hypothetical protein DKFZp434A128 | DKFZP434A128 |
| 219876_s_at | hypothetical protein DKFZp434M0331 | DKFZP434M0331 |
| 213657_s_at | Hypothetical protein DKFZp547K1113 | DKFZP547K1113 |
| 37590_g_at | Hypothetical protein DKFZp547K1113 | DKFZP547K1113 |
| 1556158_at | hypothetical protein DKFZp666G057 | DKFZP666G057 |
| 226018_at | hypothetical protein Ells1 | ELLS1 |
| 218824_at | hypothetical protein FLJ10781 | FLJ10781 |
| 218999_at | hypothetical protein FLJ11000 | FLJ11000 |
| 243465_at | Hypothetical protein FLJ11000 | FLJ11000 |
| 218627_at | hypothetical protein FLJ11259 | FLJ11259 |
| 1555491_a_at | hypothetical protein FLJ11286 | FLJ11286 |
| 53720_at | hypothetical protein FLJ11286 | FLJ11286 |
| 220156_at | hypothetical protein FLJ11767 | EFCAB1 |
| 220361_at | hypothetical protein FLJ12476 | FLJ12476 |
| 219381_at | hypothetical protein FLJ13231 | FLJ13231 |
| 221908_at | Hypothetical protein FLJ14627 | FLJ14627 |
| 221909_at | hypothetical protein FLJ14627 | FLJ14627 |
| 218986_s_at | hypothetical protein FLJ20035 | FLJ20035 |
| 218532_s_at | hypothetical protein FLJ20152 | FLJ20152 |
| 236276_at | Hypothetical protein FLJ20366 | FLJ20366 |
| 218802_at | hypothetical protein FLJ20647 | FLJ20647 |
| 219895_at | hypothetical protein FLJ20716 | FLJ20716 |
| 1554919_s_at | hypothetical protein FLJ21062 | FLJ21062 |
| 219455_at | hypothetical protein FLJ21062 | FLJ21062 |
| 219334_s_at | hypothetical protein FLJ22833 | FLJ22833 |
| 1554140_at | hypothetical protein FLJ23129 | WDR78 |
| 1554141_s_at | hypothetical protein FLJ23129 | WDR78 |
| 220389_at | hypothetical protein FLJ23514 | FLJ23514 |
| 215341_at | hypothetical protein FLJ23529 | FLJ23529 |
| 237220_at | Hypothetical protein FLJ23834 | FLJ23834 |
| 228152_s_at | hypothetical protein FLJ31033 | FLJ31033 |
| 230339_at | Hypothetical protein FLJ32745 | FLJ32745 |
| 230047_at | hypothetical protein FLJ32810 | FLJ32810 |
| 233157_x_at | hypothetical protein FLJ32926 | FLJ32926 |
| 230158_at | hypothetical protein FLJ32949 | DPY19L2 |
| 1558899_s_at | hypothetical protein FLJ35946 | FLJ35946 |
| 236418_at | hypothetical protein FLJ36119 | TTLL5 |
| 215143_at | Hypothetical protein FLJ36166 | FLJ36166 |
| 1553314_a_at | hypothetical protein FLJ37300 | FLJ37300 |
| 1553362_at | hypothetical protein FLJ37357 | FLJ37357 |
| 228903_at | hypothetical protein FLJ37464 | FLJ37464 |
| 242470_at | hypothetical protein FLJ38944 | FLJ38944 |
| 1552389_at | hypothetical protein FLJ39553 | FLJ39553 |
| 1552390_a_at | hypothetical protein FLJ39553 | FLJ39553 |
| 231081_at | hypothetical protein FLJ40298 | FLJ40298 |
| 228100_at | hypothetical protein LOC128344 | C1ORF88 |
| 226702_at | hypothetical protein LOC129607 | LOC129607 |
| 239722_at | hypothetical protein LOC134121 | LOC134121 |
| 1557636_a_at | hypothetical protein LOC136288 | LOC136288 |
| 228863_at | Hypothetical protein LOC144997 | PCDH17 |
| 239593_at | hypothetical protein LOC155006 | LOC155006 |
| 241416_at | Hypothetical protein LOC155036 | LOC155036 |
| 1556357_s_at | hypothetical protein LOC157697 | LOC157697 |
| 238625_at | hypothetical protein LOC199920 | C1ORF168 |
| 213248_at | hypothetical protein LOC221362 | LOC221362 |
| 1557417_s_at | hypothetical protein LOC222967 | LOC222967 |
| 242601_at | hypothetical protein LOC253012 | LOC253012 |
| 236076_at | hypothetical protein LOC257396 | LOC257396 |
| 213148_at | hypothetical protein LOC257407 | LOC257407 |
| 1556062_at | hypothetical protein LOC283012 | LOC283012 |
| 1561096_at | hypothetical protein LOC285419 | LOC285419 |
| 1562209_at | hypothetical protein LOC285429 | LOC285429 |
| 232504_at | hypothetical protein LOC285628 | LOC285628 |
| 1557107_at | hypothetical protein LOC286002 | LOC286002 |
| 232921 _at | hypothetical protein LOC286025 | LOC286025 |
| 225033_at | hypothetical protein LOC286167 | LOC286167 |
| 206721_at | hypothetical protein LOC57821 | C1ORF114 |
| 227910_at | hypothetical protein LOC63929 | DNAJB7 |
| 232611_at | hypothetical protein LOC92497 | LOC92497 |
| 212281_s_at | hypothetical protein MAC30 | MAC30 |
| 224495_at | hypothetical protein MGC10744 | MGC10744 |
| 224463_s_at | hypothetical protein MGC13040 | MGC13040 |
| 1563863_x_at | hypothetical protein MGC17403 | MGC17403 |
| 1553055_a_at | hypothetical protein MGC19764 | MGC19764 |
| 235498_at | hypothetical protein MGC22773 | LRRC44 |
| 228532_at | hypothetical protein MGC24133 | C1ORF162 |
| 236085_at | hypothetical protein MGC26610 | CAPSL |
| 1561200_at | hypothetical protein MGC26733 | MGC26733 |
| 228606_at | hypothetical protein MGC33212 | MGC33212 |
| 1555007_s_at | hypothetical protein MGC33630 | WDR66 |
| 229302_at | hypothetical protein MGC33926 | MGC33926 |
| 1553729_s_at | hypothetical protein MGC35140 | LRRC43 |
| 231549_at | hypothetical protein MGC35194 | C1ORF158 |
| 238008_at | hypothetical protein MGC35308 | MGC35308 |
| 237020_at | hypothetical protein MGC39581 | MGC39581 |
| 243832_at | Hypothetical protein MGC5391 | SFT2D3 |
| 221477_s_at | hypothetical protein MGC5618 | MGC5618 |
| 235743_at | Hypothetical protein MGC61716 | MTERFD2 |
| 213038_at | IBR domain containing 3 | IBRDC3 |
| 36564_at | IBR domain containing 3 | IBRDC3 |
| 230670_at | immunoglobulin superfamily, member 10 | IGSF10 |
| 210029_at | indoleamine-pyrrole 2,3 dioxygenase | INDO |
| 215177_s_at | integrin, alpha 6 | ITGA6 |
| 1555349_a_at | integrin, beta 2 (antigen CD18 (p95), lymphocyte function- | ITGB2 |
| | associated antigen 1; macrophage antigen 1 (mac-1) beta | |
| | subunit) | |
| 208083_s_at | integrin, beta 6 | ITGB6 |
| 226535_at | integrin, beta 6 | ITGB6 |
| 208084_at | integrin, beta 6 | ITGB6 |
| 202637_s_at | intercellular adhesion molecule 1 (CD54), human rhinovirus | ICAM1 |
| | receptor | |
| 202638_s_at | intercellular adhesion molecule 1 (CD54), human rhinovirus | ICAM1 |
| | receptor | |
| 215485_s_at | intercellular adhesion molecule 1 (CD54), human rhinovirus | ICAM1 |
| | receptor | |
| 201601_x_at | interferon induced transmembrane protein 1 (9-27) | IFITM1 |
| 214022_s_at | interferon induced transmembrane protein 1 (9-27) | IFITM1 |
| 201315_x_at | interferon induced transmembrane protein 2 (1-8D) | IFITM2 |
| 212203_x_at | interferon induced transmembrane protein 3 (1-8U) | IFITM3 |
| 1555464_at | interferon induced with helicase C domain 1 | IFIH1 |
| 216020_at | Interferon induced with helicase C domain 1 | IFIH1 |
| 219209_at | interferon induced with helicase C domain 1 | IFIH1 |
| 202531_at | interferon regulatory factor 1 | IRF1 |
| 208436_s_at | interferon regulatory factor 7 | IRF7 |
| 204057_at | interferon regulatory factor 8 | IRF8 |
| 204698_at | interferon stimulated gene 20 kDa | ISG20 |
| 33304_at | interferon stimulated gene 20 kDa | ISG20 |
| 205483_s_at | interferon, alpha-inducible protein (clone IFI-15K) | G1P2 |
| 204415_at | interferon, alpha-inducible protein (clone IFI-6-16) | G1P3 |
| 210354_at | interferon, gamma | IFNG |
| 209417_s_at | interferon-induced protein 35 | IFI35 |
| 214059_at | Interferon-induced protein 44 | IFI44 |
| 214453_s_at | interferon-induced protein 44 | IFI44 |
| 204439_at | interferon-induced protein 44-like | IFI44L |
| 203153_at | interferon-induced protein with tetratricopeptide repeats 1 | IFIT1 |
| 217502_at | interferon-induced protein with tetratricopeptide repeats 2 | IFIT2 |
| 226757_at | interferon-induced protein with tetratricopeptide repeats 2 | IFIT2 |
| 204747_at | interferon-induced protein with tetratricopeptide repeats 3 | IFIT3 |
| 229450_at | interferon-induced protein with tetratricopeptide repeats 3 | IFIT3 |
| 203595_s_at | interferon-induced protein with tetratricopeptide repeats 5 | IFIT5 |
| 203596_s_at | interferon-induced protein with tetratricopeptide repeats 5 | IFIT5 |
| 212657_s_at | interleukin 1 receptor antagonist | IL1RN |
| 39402_at | interleukin 1, beta | IL1B |
| 207433_at | interleukin 10 | IL10 |
| 207375_s_at | interleukin 15 receptor, alpha | IL15RA |
| 1555016_at | interleukin 16 (lymphocyte chemoattractant factor) | IL16 |
| 209827_s_at | interleukin 16 (lymphocyte chemoattractant factor) | IL16 |
| 222868_s_at | interleukin 18 binding protein | IL18BP |
| 220745_at | interleukin 19 | IL19 |
| 1552609_s_at | interleukin 28A (interferon, lambda 2) | IL28A |
| 203828_s_at | interleukin 32 | IL32 |
| 230966_at | interleukin 4 induced 1 | IL4I1 |
| 210744_s_at | interleukin 5 receptor, alpha | IL5RA |
| 205207_at | interleukin 6 (interferon, beta 2) | IL6 |
| 206693_at | interleukin 7 | IL7 |
| 241808_at | Interleukin 7 | IL7 |
| 226218_at | Interleukin 7 receptor | IL7R |
| 202859_x_at | interleukin 8 | IL8 |
| 211506_s_at | interleukin 8 | IL8 |
| 233290_at | Interleukin-1 receptor-associated kinase 1 binding protein 1 | IRAK1BP1 |
| 1554739_at | intracisternal A particle-promoted polypeptide | IPP |
| 236235_at | Itchy homolog E3 ubiquitin protein ligase (mouse) | ITCH |
| 205841_at | Janus kinase 2 (a protein tyrosine kinase) | JAK2 |
| 205842_s_at | Janus kinase 2 (a protein tyrosine kinase) | JAK2 |
| 227677_at | Janus kinase 3 (a protein tyrosine kinase, leukocyte) | JAK3 |
| 229294_at | junctophilin 3 | JPH3 |
| 205157_s_at | keratin 17 | KRT17 |
| 209125_at | keratin 6A | KRT6C |
| 204166_at | KIAA0963 | KIAA0963 |
| 212942_s_at | KIAA1199 | KIAA1199 |
| 1562648_at | KIAA1212 | KIAA1212 |
| 234936_s_at | KIAA1345 protein | KIAA1345 |
| 225076_s_at | KIAA1404 protein | KIAA1404 |
| 1569503_at | KIAA1414 protein | KIAA1414 |
| 227409_at | KIAA1443 | KIAA1443 |
| 222139_at | KIAA1466 gene | KIAA1466 |
| 241347_at | KIAA1618 | KIAA1618 |
| 225193_at | KIAA1967 | KI1AA1967 |
| 203934_at | kinase insert domain receptor (a type III receptor tyrosine | KDR |
| | kinase) | |
| 223778_at | kinesin family member 9 | KIF9 |
| 228429_x_at | kinesin family member 9 | KIF9 |
| 231319_x_at | kinesin family member 9 | KIF9 |
| 204385_at | kynureninase (L-kynurenine hydrolase) | KYNU |
| 210663_s_at | kynureninase (L-kynurenine hydrolase) | KYNU |
| 217388_s_at | kynureninase (L-kynurenine hydrolase) | KYNU |
| 205306_x_at | kynurenine 3-monooxygenase (kynurenine 3-hydroxylase) | KMO |
| 203276_at | lamin B1 | LMNB1 |
| 217933_s_at | leucine aminopeptidase 3 | LAP3 |
| 236917_at | leucine rich repeat containing 34 | LRRC34 |
| 236918_s_at | leucine rich repeat containing 34 | LRRC34 |
| 220003_at | leucine rich repeat containing 36 | LRRC36 |
| 205266_at | leukemia inhibitory factor (cholinergic differentiation factor) | LIF |
| 205876_at | leukemia inhibitory factor receptor | LIFR |
| 225571_at | Leukemia inhibitory factor receptor | LIFR |
| 227771_at | Leukemia inhibitory factor receptor | LIFR |
| 225575_at | leukemia inhibitory factor receptor | lifr |
| 210660_at | leukocyte imunoglobulin-like receptor, subfamily A (with | LILRA1 |
| | TM domain), member 1 | |
| 211100_x_at | leukocyte immunoglobulin-like receptor, subfamily A (with | LILRA2 |
| | TM domain), member 2 | |
| 207104_x_at | leukocyte immunoglobulin-like receptor, subfamily B (with | LILRB1 |
| | TM and ITIM domains), member 1 | |
| 229937_x_at | Leukocyte immunoglobulin-like receptor, subfamily B (with | LILRB1 |
| | TM and ITIM domains), member 1 | |
| 207697_x_at | leukocyte immunoglobulin-like receptor, subfamily B (with | LILRB2 |
| | TM and ITIM domains), member 2 | |
| 210225_x_at | leukocyte immunoglobulin-like receptor, subfamily B (with | LILRB2 |
| | TM and ITIM domains), member 2 | |
| 210784_x_at | leukocyte immunoglobulin-like receptor, subfamily B (with | LILRB2 |
| | TM and ITIM domains), member 2 | |
| 211135_x_at | leukocyte immunoglobulin-like receptor, subfamily B (with | LILRB2 |
| | TM and ITIM domains), member 2 | |
| 208594_x_at | leukocyte immunoglobulin-like receptor, subfamily B (with | LILRB3 |
| | TM and ITIM domains), member 6 | |
| 215838_at | leukocyte immunoglobulin-like receptor, subfamily B (with | LILRA5 |
| | TM and ITIM domains), member 7 | |
| 210644_s_at | leukocyte-associated Ig-like receptor 1 | LAIR1 |
| 224806_at | LOC440448 | LOC440448 |
| 230552_at | LOC440523 | LOC440523 |
| 239279_at | LOC440702 | LOC440702 |
| 237585_at | LOC441054 | LOC441054 |
| 236045_x_at | LOC441801 | LOC441801 |
| 202067_s_at | low density lipoprotein receptor (familial | LDLR |
| | hypercholesterolemia) | |
| 217173_s_at | low density lipoprotein receptor (familial | LDLR |
| | hypercholesterolemia) | |
| 202068_s_at | low density lipoprotein receptor (familial | LDLR |
| | hypercholesterolemia) | |
| 235126_at | LQK1 hypothetical protein short isoform (LQK1) mRNA, | |
| | complete cds, alternatively spliced | |
| 220532_s_at | LR8 protein | LR8 |
| 207797_s_at | LRP2 binding protein | LRP2BP |
| 206486_at | lymphocyte-activation gene 3 | LAG3 |
| 205569_at | lysosomal-associated membrane protein 3 | LAMP3 |
| 209728_at | major histocompatibility complex, class II, DR beta 4 | HLA-DRB4 |
| 204475_at | matrix metalloproteinase 1 (interstitial collagenase) | MMP1 |
| 204580_at | matrix metalloproteinase 12 (macrophage elastase) | MMP12 |
| 202827_s_at | matrix metalloproteinase 14 (membrane-inserted) | MMP14 |
| 217279_x_at | matrix metalloproteinase 14 (membrane-inserted) | MMP14 |
| 203936_s_at | matrix metalloproteinase 9 (gelatinase B, 92 kDa gelatinase, | MMP9 |
| | 92 kDa type IV collagenase) | |
| 218810_at | MCP-1 treatment-induced protein | ZC3H12A |
| 1552594_at | MDAC1 | MDAC1 |
| 205655_at | Mdm4, transformed 3T3 cell double minute 4, p53 binding | MDM4 |
| | protein (mouse) | |
| 241876_at | Mdm4, transformed 3T3 cell double minute 4, p53 binding | MDM4 |
| | protein (mouse) | |
| 219703_at | meiosis-specific nuclear structural protein 1 | MNS1 |
| 221369_at | melatonin receptor 1A | MTNR1A |
| 219574_at | membrane-associated ring finger (C3HC4) 1 | 1-Mar |
| 229383_at | Membrane-associated ring finger (C3HC4) 1 | 1-Mar |
| 219607_s_at | membrane-spanning 4-domains, subfamily A, member 4 | MS4A4A |
| 212185_x_at | metallothionein 2A | MT2A |
| 201761_at | methylenetetrahydrofolate dehydrogenase (NADP+ | MTHFD2 |
| | dependent) 2, methenyltetrahydrofolate cyclohydrolase | |
| 205101_at | MHC class II transactivator | MHC2TA |
| 226084_at | microtubule-associated protein 1B | MAP1B |
| 228943_at | microtubule-associated protein 6 | MAP6 |
| 1552573_s_at | mirror-image polydactyly 1 | MIPOL1 |
| 222528_s_at | mitochondrial solute carrier protein | SLC25A37 |
| 238025_at | mixed lineage kinase domain-like | MLKL |
| 204041_at | monoamine oxidase B | MAOB |
| 215731_s_at | M-phase phosphoprotein 9 | MPHOSPH9 |
| 1561272_at | MRNA; cDNA DKFZp313J0134 (from clone | |
| | DKFZp313J0134) | |
| 225812_at | MRNA; cDNA DKFZp586F0922 (from clone | |
| | DKFZp586F0922) | |
| 1568698_at | MRNA; cDNA DKFZp686G0585 (from clone | |
| | DKFZp686G0585) | |
| 238484_s_at | MRNA; clone CD 43T7 | |
| 238752_at | MRS2-like, magnesium homeostasis factor (S. cerevisiae) | MRS2L |
| 222712_s_at | mucin 13, epithelial transmembrane | MUC13 |
| 218687_s_at | mucin 13, epithelial transmembrane | MUC13 |
| 227241_at | mucin 15 | MUC15 |
| 235740_at | Multiple C2-domains with two transmembrane regions 1 | MCTP1 |
| 213306_at | multiple PDZ domain protein | MPDZ |
| 212913_at | mutS homolog 5 (E. coli) | MSH5 |
| 200798_x_at | myeloid cell leukemia sequence 1 (BCL2-related) | MCL1 |
| 200796_s_at | myeloid cell leukemia sequence 1 (BCL2-related) | MCL1 |
| 200797_s_at | myeloid cell leukemia sequence 1 (BCL2-related) | MCL1 |
| 202086_at | myxovirus (influenza virus) resistance 1, interferon-inducible | MX1 |
| | protein p78 (mouse) | |
| 204994_at | myxovirus (influenza virus) resistance 2 (mouse) | MX2 |
| 206418_at | NADPH oxidase 1 | NOX1 |
| 213915_at | natural killer cell group 7 sequence | NKG7 |
| 1557071_s_at | NEDD8 ultimate buster-1 | NYREN18 |
| 238844_s_at | nephronophthisis 1 (juvenile) | NPHP1 |
| 1552309_a_at | nexilin (F actin binding protein) | NEXN |
| 226103_at | nexilin (F actin binding protein) | NEXN |
| 211086_x_at | NIMA (never in mitosis gene a)-related kinase 1 | NEK1 |
| 210037_s_at | nitric oxide synthase 2A (inducible, hepatocytes) | NOS2A |
| 200632_s_at | N-myc downstream regulated gene 1 | NDRG1 |
| 206197_at | non-metastatic cells 5, protein expressed in (nucleoside- | NME5 |
| | diphosphate kinase) | |
| 210218_s_at | nuclear antigen Sp100 | SP100 |
| 209636_at | nuclear factor of kappa light polypeptide gene enhancer in B- | NFKB2 |
| | cells 2 (p49/p100) | |
| 201502_s_at | nuclear factor of kappa light polypeptide gene enhancer in B- | NFKBIA |
| | cells inhibitor, alpha | |
| 223217_s_at | nuclear factor of kappa light polypeptide gene enhancer in B- | NFKBIZ |
| | cells inhibitor, zeta | |
| 223218_s_at | nuclear factor of kappa light polypeptide gene enhancer in B- | NFKBIZ |
| | cells inhibitor, zeta | |
| 241031_at | Nuclear localized factor 1 | NLF1 |
| 225344_at | nuclear receptor coactivator 7 | NCOA7 |
| 205729_at | oncostatin M receptor | OSMR |
| 226621_at | Oncostatin M receptor | OSMR |
| 210415_s_at | outer dense fiber of sperm tails 2 | ODF2 |
| 238575_at | oxysterol binding protein-like 6 | OSBPL6 |
| 209230_s_at | p8 protein (candidate of metastasis 1) | P8 |
| 222725_s_at | palmdelphin | PALMD |
| 1556773_at | Parathyroid hormone-like hormone | PTHLH |
| 206300_s_at | parathyroid hormone-like hormone | PTHLH |
| 214204_at | PARK2 co-regulated | PACRG |
| 234927_s_at | PDZ domain containing, X chromosome | FLJ21687 |
| 236548_at | PDZ domain protein GIPC2 | GIPC2 |
| 1553589_a_at | PDZK1 interacting protein 1 | PDZK1IP1 |
| 219630_at | PDZK1 interacting protein 1 | PDZK1IP1 |
| 1553681_a_at | perforin 1 (pore forming protein) | PRF1 |
| 214617_at | perforin 1 (pore forming protein) | PRF1 |
| 224210_s_at | peroxisomal membrane protein 4, 24 kDa | PXMP4 |
| 228230_at | peroxisomal proliferator-activated receptor A interacting | PRIC285 |
| | complex 285 | |
| 232517_s_at | peroxisomal proliferator-activated receptor A interacting | PRIC285 |
| | complex 285 | |
| 219195_at | peroxisome proliferative activated receptor, gamma, | PPARGC1A |
| | coactivator 1, alpha | |
| 204285_s_at | phorbol-12-myristate-13-acetate-induced protein 1 | PMAIP1 |
| 204286_s_at | phorbol-12-myristate-13-acetate-induced protein 1 | PMAIP1 |
| 232553_at | phosphate cytidylyltransferase 1, choline, beta isoform | PCYT1B |
| 239808_at | Phosphatidylinositol transfer protein, cytoplasmic 1 | PITPNC1 |
| 1558680_s_at | phosphodiesterase 1A, calmodulin-dependent | PDE1A |
| 231213_at | phosphodiesterase 1A, calmodulin-dependent | PDE1A |
| 226459_at | phosphoinositide-3-kinase adaptor protein 1 | PIK3AP1 |
| 202430_s_at | phospholipid scramblase 1 | PLSCR1 |
| 202446_s_at | phospholipid scramblase 1 | PLSCR1 |
| 241916_at | Phospholipid scramblase 1 | PLSCR1 |
| 218901_at | phospholipid scramblase 4 | PLSCR4 |
| 1558534_at | PI-3-kinase-related kinase SMG-1-like | DKFZP547E087 |
| 211924_s_at | plasminogen activator, urokinase receptor | PLAUR |
| 203470_s_at | pleckstrin | PLEK |
| 203471_s_at | pleckstrin | PLEK |
| 218613_at | pleckstrin and Sec7 domain containing 3 | PSD3 |
| 1557363_a_at | pleckstrin homology domain interacting protein | PHIP |
| 224701_at | poly (ADP-ribose) polymerase family, member 14 | PARP14 |
| 235157_at | Poly (ADP-ribose) polymerase family, member 14 | PARP14 |
| 223220_s_at | poly (ADP-ribose) polymerase family, member 9 | PARP9 |
| 227807_at | Poly (ADP-ribose) polymerase family, member 9 | PARP9 |
| 225291_at | polyribonucleotide nucleotidyltransferase 1 | PNPT1 |
| 1555167_s_at | pre-B-cell colony enhancing factor 1 | PBEF1 |
| 207838_x_at | pre-B-cell leukemia transcription factor interacting protein 1 | PBXIP1 |
| 235229_at | PREDICTED: Homo sapiens similar to Olfactory receptor 2I2 | |
| | (LOC442197), mRNA | |
| 238531_x_at | PREDICTED: Homo sapiens similar to Olfactory receptor 2I2 | |
| | (LOC442197), mRNA | |
| 238629_x_at | PREDICTED: Homo sapiens similar to Olfactory receptor 2I2 | |
| | (LOC442197), mRNA | |
| 231077_at | PREDICTED: Homo sapiens similar to RIKEN cDNA | |
| | 1700009P17 (LOC257177), mRNA | |
| 230044_at | Preproneuropeptide B | NPB |
| 227458_at | programmed cell death 1 ligand 1 | PDCD111 |
| 223834_at | programmed cell death 1 ligand 1 | PDCD111 |
| 206503_x_at | promyelocytic leukemia | PML |
| 209640_at | promyelocytic leukemia | PML |
| 210362_x_at | promyelocytic leukemia | PML |
| 211012_s_at | promyelocytic leukemia | PML |
| 211013_x_at | promyelocytic leukemia | PML |
| 211588_s_at | promyelocytic leukemia | PML |
| 235508_at | Promyelocytic leukemia | PML |
| 213652_at | Proprotein convertase subtilisin/kexin type 5 | PCSK5 |
| 204748_at | prostaglandin-endoperoxide synthase 2 (prostaglandin G/H | PTGS2 |
| | synthase and cyclooxygenase) | |
| 218083_at | prostaglandin E synthase 2 | PTGES2 |
| 1555097_a_at | prostaglandin F receptor (FP) | PTGFR |
| 207177_at | prostaglandin F receptor (FP) | PTGFR |
| 222277_at | prostate collagen triple helix | PCOTH |
| 226279_at | protease, serine, 23 | PRSS23 |
| 229441_at | Protease, serine, 23 | PRSS23 |
| 204211_x_at | protein kinase, interferon-inducible double stranded RNA | PRKR |
| | dependent | |
| 237105_at | protein kinase, interferon-inducible double stranded RNA | PRKRA |
| | dependent activator | |
| 228620_at | protein kinase, interferon-inducible double stranded RNA | PRKRA |
| | dependent activator | |
| 218273_s_at | protein phosphatase 2C, magnesium-dependent, catalytic | PPM2C |
| | subunit | |
| 222572_at | protein phosphatase 2C, magnesium-dependent, catalytic | PPM2C |
| | subunit | |
| 207808_s_at | protein S (alpha) | PROS1 |
| 1569552_at | protein tyrosine phosphatase, non-receptor type 18 (brain- | PTPN18 |
| | derived) | |
| 208300_at | protein tyrosine phosphatase, receptor type, H | PTPRH |
| 203030_s_at | protein tyrosine phosphatase, receptor type, N polypeptide 2 | PTPRN2 |
| 209323_at | protein-kinase, interferon-inducible double stranded RNA | PRKRIR |
| | dependent inhibitor, repressor of (P58 repressor) | |
| 227289_at | protocadherin 17 | PCDH17 |
| 205656_at | protocadherin 17 | PCDH17 |
| 227282_at | protocadherin 19 | PCDH19 |
| 238117_at | protoporphyrinogen oxidase | PPOX |
| 204788_s_at | protoporphyrinogen oxidase | ppox |
| 220005_at | purinergic receptor P2Y, G-protein coupled, 13 | P2RY13 |
| 206637_at | purinergic receptor P2Y, G-protein coupled, 14 | P2RY14 |
| 1563104_at | RAB11 family interacting protein 3 (class II) | RAB11FIP3 |
| 205925_s_at | RAB3B, member RAS oncogene family | RAB3B |
| 227123_at | RAB3B, member RAS oncogene family | RAB3B |
| 239202_at | RAB3B, member RAS oncogene family | RAB3B |
| 213797_at | radical S-adenosyl methionine domain containing 2 | RSAD2 |
| 242625_at | radical S-adenosyl methionine domain containing 2 | RSAD2 |
| 226436_at | Ras association (RalGDS/AF-6) domain family 4 | RASSF4 |
| 209545_s_at | receptor-interacting serine-threonine kinase 2 | RIPK2 |
| 1555804_a_at | regulated in COPD kinase | YSK4 |
| 202988_s_at | regulator of G-protein signalling 1 | RGS1 |
| 223691_at | regulator of G-protein signalling 22 | RGS22 |
| 220105_at | rhabdoid tumor deletion region gene 1 | RTDR1 |
| 206526_at | RIB43A domain with coiled-coils 2 | RIBC2 |
| 206111_at | ribonuclease, RNase A family, 2 (liver, eosinophil-derived | RNASE2 |
| | neurotoxin) | |
| 242442_x_at | RNA (guanine-9-) methyltransferase domain containing 2 | RG9MTD2 |
| 238763_at | RNA binding motif protein 20 | RBM20 |
| 235004_at | RNA binding motif protein 24 | RBM24 |
| 223609_at | ropporin 1-like | ROPN1L |
| 220330_s_at | SAM domain, SH3 domain and nuclear localisation signals, 1 | SAMSN1 |
| 213435_at | SATB family member 2 | SATB2 |
| 223843_at | scavenger receptor class A, member 3 | SCARA3 |
| 213456_at | sclerostin domain containing 1 | SOSTDC1 |
| 205241_at | SCO cytochrome oxidase deficient homolog 2 (yeast) | SCO2 |
| 216346_at | SEC14-like 3 (S. cerevisiae) | SEC14L3 |
| 240699_at | SEC14-like 3 (S. cerevisiae) | SEC14L3 |
| 213716_s_at | secreted and transmembrane 1 | SECTM1 |
| 209875_s_at | secreted phosphoprotein 1 (osteopontin, bone sialoprotein I, | SPP1 |
| | early T-lymphocyte activation 1) | |
| 204563_at | selectin L (lymphocyte adhesion molecule 1) | SELL |
| 228869_at | Selectin ligand interactor cytoplasmic-1 | SLIC1 |
| 231669_at | Selenoprotein P, plasma, 1 | SEPP1 |
| 217977_at | selenoprotein X, 1 | SEPX1 |
| 215028_at | sema domain, transmembrane domain (TM), and cytoplasmic | SEMA6A |
| | domain, (semaphorin) 6A | |
| 225660_at | sema domain, transmembrane domain (TM), and cytoplasmic | SEMA6A |
| | domain, (semaphorin) 6A | |
| 223567_at | sema domain, transmembrane domain (TM), and cytoplasmic | SEMA6B |
| | domain, (semaphorin) 6B | |
| 202376_at | serine (or cysteine) proteinase inhibitor, clade A (alpha-1 | SERPINA3 |
| | antiproteinase, antitrypsin), member 3 | |
| 212268_at | serine (or cysteine) proteinase inhibitor, clade B (ovalbumin), | SERPINB1 |
| | member 1 | |
| 239213_at | Serine (or cysteine) proteinase inhibitor, clade B (ovalbumin), | SERPINB1 |
| | member 1 | |
| 1552463_at | serine (or cysteine) proteinase inhibitor, clade B (ovalbumin), | SERPINB11 |
| | member 11 | |
| 1563357_at | Serine (or cysteine) proteinase inhibitor, clade B (ovalbumin), | SERPINB9 |
| | member 9 | |
| 209723_at | serine (or cysteine) proteinase inhibitor, clade B (ovalbumin), | SERPINB9 |
| | member 9 | |
| 242814_at | serine (or cysteine) proteinase inhibitor, clade B (ovalbumin), | SERPINB9 |
| | member 9 | |
| 200986_at | serine (or cysteine) proteinase inhibitor, clade G (C1 | SERPING1 |
| | inhibitor), member 1, (angioedema, hereditary) | |
| 206319_s_at | serine protease inhibitor-like, with Kunitz and WAP domains | SPINLW1 |
| | 1 (eppin) | |
| 228035_at | serine/threonine kinase 33 | STK33 |
| 208607_s_at | serum amyloid A1 | SAA1 |
| 214456_x_at | serum amyloid A1 | SAA1 |
| 207096_at | serum amyloid A4, constitutive | SAA4 |
| 222717_at | serum deprivation response (phosphatidylserine binding | SDPR |
| | protein) | |
| 44673_at | sialoadhesin | SN |
| 208322_s_at | sialyltransferase 4A (beta-galactoside alpha-2,3- | SIAT4A |
| | sialyltransferase) | |
| 209969_s_at | signal transducer and activator of transcription 1, 91 kDa | STAT1 |
| 232375_at | Signal transducer and activator of transcription 1, 91 kDa | STAT1 |
| 205170_at | signal transducer and activator of transcription 2, 113 kDa | STAT2 |
| 217199_s_at | signal transducer and activator of transcription 2, 113 kDa | STAT2 |
| 225636_at | signal transducer and activator of transcription 2, 113 kDa | STAT2 |
| 206181_at | signaling lymphocytic activation molecule family member 1 | SLAMF1 |
| 1559760_at | Similar to ankyrin repeat domain 20A | LOC442146 |
| 226612_at | similar to CG4502-PA | FLJ25076 |
| 231044_at | similar to CG5435-PA | LOC127003 |
| 1560118_at | Similar to cysteine and histidine-rich domain (CHORD)- | LOC388943 |
| | containing, zinc-binding protein 1 | |
| 1554609_at | similar to Cytochrome c, somatic | MGC12965 |
| 230314_at | Similar to hypothetical protein 628 | LOC440424 |
| 239150_at | similar to hypothetical protein A430083B19 | LOC132203 |
| 231923_at | Similar to hypothetical protein LOC231503 | LOC441027 |
| 230033_at | similar to hypothetical testis protein from macaque | LOC352909 |
| 241912_at | Similar to Hypothetical zinc finger protein KIAA1956 | LOC400721 |
| 240287_at | similar to immune-responsive gene 1 | LOC341720 |
| 1570541_s_at | Similar to Interferon-induced guanylate-binding protein 1 | LOC400759 |
| | (GTP-binding protein 1) (Guanine nucleotide-binding protein | |
| | 1) (HuGBP-1) | |
| 216565_x_at | similar to Interferon-induced transmembrane protein 3 | LOC391020 |
| | (Interferon-inducible protein 1-8U) | |
| 236666_s_at | Similar to leucine rich repeat containing 10 | LOC390205 |
| 227522_at | similar to mouse 2310016A09Rik gene | LOC134147 |
| 230615_at | similar to Numb-interacting homolog gene | LOC405753 |
| 237291_at | similar to RIKEN cDNA 2010316F05 | LOC344405 |
| 227628_at | similar to RIKEN cDNA 2310016C16 | LOC493869 |
| 242555_at | similar to RIKEN cDNA 4921524J17 | LOC388272 |
| 222068_s_at | similar to RIKEN cDNA 4930457P18 | LRRC50 |
| 228362_s_at | Similar to RIKEN cDNA A630077B13 gene; RIKEN cDNA | LOC441168 |
| | 2810048G17 | |
| 229390_at | similar to RIKEN cDNA A630077B13 gene; RIKEN cDNA | LOC441168 |
| | 2810048G17 | |
| 229391_s_at | similar to RIKEN cDNA A630077B13 gene; RIKEN cDNA | LOC441168 |
| | 2810048G17 | |
| 230591_at | Similar to Serine/threonine-protein kinase PLK1 (Polo-like | LOC441777 |
| | kinase 1) (PLK-1) (Serine-threonine protein kinase 13) | |
| | (STPK13) | |
| 1559681_a_at | Similar to tripartite motif-containing 16; estrogen-responsive | LOC147166 |
| | B box protein | |
| 244551_at | Similar to zinc finger protein 92 (HTF12) | LOC442699 |
| 219159_s_at | SLAM family member 7 | SLAMF7 |
| 222838_at | SLAM family member 7 | SLAMF7 |
| 234306_s_at | SLAM family member 7 | SLAMF7 |
| 219386_s_at | SLAM family member 8 | SLAMF8 |
| 232547_at | SNAP25-interacting protein | SNIP |
| 241436_at | sodium channel, nonvoltage-gated 1, gamma | SCNN1G |
| 243713_at | Solute carrier family 1 (neuronal/epithelial high affinity | SLC1A1 |
| | glutamate transporter, system Xag), member 1 | |
| 219593_at | solute carrier family 15, member 3 | SLC15A3 |
| 1557918_s_at | solute carrier family 16 (monocarboxylic acid transporters), | SLC16A1 |
| | member 1 | |
| 202236_s_at | solute carrier family 16 (monocarboxylic acid transporters), | SLC16A1 |
| | member 1 | |
| 209900_s_at | solute carrier family 16 (monocarboxylic acid transporters), | SLC16A1 |
| | member 1 | |
| 202497_x_at | solute carrier family 2 (facilitated glucose transporter), | SLC2A3 |
| | member 3 | |
| 216236_s_at | solute carrier family 2 (facilitated glucose transporter), | SLC2A14 |
| | member 3 | |
| 1554161_at | solute carrier family 25, member 27 | SLC25A27 |
| 1560705_at | Solute carrier family 25, member 28 | SLC25A28 |
| 221432_s_at | solute carrier family 25, member 28 | SLC25A28 |
| 223192_at | solute carrier family 25, member 28 | SLC25A28 |
| 206529_x_at | solute carrier family 26, member 4 | SLC26A4 |
| 232277_at | Solute carrier family 28 (sodium-coupled nucleoside | SLC28A3 |
| | transporter), member 3 | |
| 204204_at | solute carrier family 31 (copper transporters), member 2 | SLC31A2 |
| 206628_at | solute carrier family 5 (sodium/glucose cotransporter), | SLC5A1 |
| | member 1 | |
| 210854_x_at | solute carrier family 6 (neurotransmitter transporter, creatine), | SLC6A8 |
| | member 8 | |
| 213843_x_at | solute carrier family 6 (neurotransmitter transporter, creatine), | SLC6A8 |
| | member 8 | |
| 237058_x_at | solute carrier family 6 (neurotransmitter transporter, GABA), | SLC6A13 |
| | member 13 | |
| 219614_s_at | solute carrier family 6 (proline IMINO transporter), member | SLC6A20 |
| | 20 | |
| 225516_at | solute carrier family 7 (cationic amino acid transporter, y+ | SLC7A2 |
| | system), member 2 | |
| 1561615_s_at | solute carrier family 8 (sodium/calcium exchanger), member 1 | SLC8A1 |
| 1554988_at | solute carrier family 9, isoform 11 | SLC9A11 |
| 218404_at | sorting nexin 10 | SNX10 |
| 208012_x_at | SP110 nuclear body protein | SP110 |
| 208392_x_at | SP110 nuclear body protein | SP110 |
| 209761_s_at | SP110 nuclear body protein | SP110 |
| 209762_x_at | SP110 nuclear body protein | SP110 |
| 223980_s_at | SP110 nuclear body protein | SP110 |
| 210033_s_at | sperm associated antigen 6 | SPAG6 |
| 206815_at | sperm associated antigen 8 | SPAG8 |
| 205406_s_at | sperm autoantigenic protein 17 | SPA17 |
| 233251_at | Spermatid perinuclear RNA binding protein | STRBP |
| 233252_s_at | spermatid perinuclear RNA binding protein | STRBP |
| 244439_at | sprouty-related, EVH1 domain containing 1 | SPRED1 |
| 204595_s_at | stanniocalcin 1 | STC1 |
| 204596_s_at | stanniocalcin 1 | STC1 |
| 204597_x_at | stanniocalcin 1 | STC1 |
| 230746_s_at | Stanniocalcin 1 | STC1 |
| 213820_s_at | START domain containing 5 | STARD5 |
| 1554923_at | sterile alpha motif domain containing 6 | SAMD6 |
| 219691_at | sterile alpha motif domain containing 9 | SAMD9 |
| 228531_at | sterile alpha motif domain containing 9 | SAMD9 |
| 218800_at | steroid 5 alpha-reductase 2-like | SRD5A2L |
| 225241_at | steroid sensitive gene 1 | URB |
| 225242_s_at | steroid sensitive gene 1 | URB |
| 243864_at | steroid sensitive gene 1 | URB |
| 203767_s_at | steroid sulfatase (microsomal), arylsulfatase C, isozyme S | STS |
| 203770_s_at | steroid sulfatase (microsomal), arylsulfatase C, isozyme S | STS |
| 243543_at | Sterol-C4-methyl oxidase-like | SC4MOL |
| 1553794_at | stomatin (EPB72)-like 3 | STOML3 |
| 1553202_at | storkhead box 1 | STOX1 |
| 229378_at | storkhead box 1 | STOX1 |
| 223939_at | succinate receptor 1 | SUCNR1 |
| 1553030_a_at | sulfite oxidase | SUOX |
| 219934_s_at | sulfotransferase family 1E, estrogen-preferring, member 1 | SULT1E1 |
| 222940_at | sulfotransferase family 1E, estrogen-preferring, member 1 | SULT1E1 |
| 215078_at | superoxide dismutase 2, mitochondrial | SOD2 |
| 215223_s_at | superoxide dismutase 2, mitochondrial | SOD2 |
| 216841_s_at | superoxide dismutase 2, mitochondrial | SOD2 |
| 221477_s_at | superoxide dismutase 2, mitochondrial | SOD2 |
| 209999_x_at | suppressor of cytokine signaling 1 | SOCS1 |
| 210001_s_at | suppressor of cytokine signaling 1 | SOCS1 |
| 213337_s_at | suppressor of cytokine signaling 1 | SOCS1 |
| 203372_s_at | suppressor of cytokine signaling 2 | SOCS2 |
| 203373_at | suppressor of cytokine signaling 2 | SOCS2 |
| 206359_at | suppressor of cytokine signaling 3 | SOCS3 |
| 206360_s_at | suppressor of cytokine signaling 3 | SOCS3 |
| 227697_at | suppressor of cytokine signaling 3 | SOCS3 |
| 210190_at | syntaxin 11 | STX11 |
| 1569566_at | TBC1 (tre-2/USP6, BUB2, cdc16) domain family, member 1 | TBC1D1 |
| 204526_s_at | TBC1 domain family, member 8 (with GRAM domain) | TBC1D8 |
| 1556318_s_at | TBP-interacting protein | CAND1 |
| 1552542_s_at | T-cell activation GTPase activating protein | TAGAP |
| 229723_at | T-cell activation GTPase activating protein | TAGAP |
| 234050_at | T-cell activation GTPase activating protein | TAGAP |
| 242388_x_at | T-cell activation GTPase activating protein | TAGAP |
| 201645_at | tenascin C (hexabrachion) | TNC |
| 216005_at | Tenascin C (hexabrachion) | TNC |
| 218864_at | tensin | TNS1 |
| 1566606_a_at | Testis expressed gene 9 | TEX9 |
| 237057_at | Testis specific, 10 | TSGA10 |
| 203824_at | tetraspanin 8 | TSPAN8 |
| 244571_s_at | Tetratricopeptide repeat domain 12 | TTC12 |
| 244190_at | THAP domain containing 5 | THAP5 |
| 201666_at | tissue inhibitor of metalloproteinase 1 (erythroid potentiating | TIMP1 |
| | activity, collagenase inhibitor) | |
| 220655_at | TNFAIP3 interacting protein 3 | TNIP3 |
| 204924_at | toll-like receptor 2 | TLR2 |
| 1552798_a_at | toll-like receptor 4 | TLR4 |
| 224341_x_at | toll-like receptor 4 | TLR4 |
| 229560_at | toll-like receptor 8 | TLR8 |
| 209593_s_at | torsin family 1, member B (torsin B) | TOR1B |
| 236833_at | torsin family 2, member A | TTC16 |
| 226117_at | TRAF-interacting protein with a forkhead-associated domain | TIFA |
| 228941_at | Transcribed locus | |
| 229278_at | Transcribed locus | |
| 229869_at | Transcribed locus | |
| 230406_at | Transcribed locus | |
| 231181_at | Transcribed locus | |
| 235670_at | Transcribed locus | |
| 236198_at | Transcribed locus | |
| 236203_at | Transcribed locus | |
| 236256_at | Transcribed locus | |
| 237573_at | Transcribed locus | |
| 238392_at | Transcribed locus | |
| 239582_at | Transcribed locus | |
| 240013_at | Transcribed locus | |
| 240183_at | Transcribed locus | |
| 240422_at | Transcribed locus | |
| 241371_at | Transcribed locus | |
| 241853_at | Transcribed locus | |
| 243063_at | Transcribed locus | |
| 243379_at | Transcribed locus | |
| 243754_at | Transcribed locus | |
| 244116_at | Transcribed locus | |
| 244313_at | Transcribed locus | |
| 235892_at | Transcribed locus, moderately similar to XP_510261.1 similar | |
| | to Gamma-tubulin complex component 5 (GCP-5) [Pan | |
| | troglodytes] | |
| 229641_at | Transcribed locus, moderately similar to XP_517655.1 similar | |
| | to KIAA0825 protein [Pan troglodytes] | |
| 230269_at | Transcribed locus, strongly similar to NP_001186.1 beaded | |
| | filament structural protein 1, filensin [Homo sapiens] | |
| 235428_at | Transcribed locus, strongly similar to XP_511361.1 similar to | |
| | ribosomal protein L23a; 60S ribosomal protein L23a; cDNA | |
| | sequence BC029892 [Pan troglodytes] | |
| 229843_at | Transcribed locus, strongly similar to XP_519844.1 similar to | |
| | CGI-90 protein [Pan troglodytes] | |
| 240182_at | Transcribed locus, strongly similar to XP_531023.1 | |
| | LOC463393 [Pan troglodytes] | |
| 230927_at | Transcribed locus, weakly similar to NP_694983.1 DHHC- | |
| | containing protein 20 [Homo sapiens] | |
| 235949_at | Transcribed locus, weakly similar to NP_775735.11(3)mbt- | |
| | like 4 (Drosophila) [Homo sapiens] | |
| 238725_at | Transcribed locus, weakly similar to XP_496299.1 | |
| | hypothetical protein LOC148206 [Homo sapiens] | |
| 235247_at | Transcription factor CP2-like 3 | TFCP2L3 |
| 232116_at | transcription factor CP2-like 4 | TFCP2L4 |
| 206715_at | transcription factor EC | TFEC |
| 232383_at | transcription factor EC | TFEC |
| 201042_at | transglutaminase 2 (C polypeptide, protein-glutamine- | TGM2 |
| | gamma-glutamyltransferase) | |
| 211573_x_at | transglutaminase 2 (C polypeptide, protein-glutamine- | TGM2 |
| | gamma-glutamyltransferase) | |
| 1554485_s_at | transmembrane protein 37 | TMEM37 |
| 227190_at | transmembrane protein 37 | TMEM37 |
| 217875_s_at | transmembrane, prostate androgen induced RNA | TMEPAI |
| 202307_s_at | transporter 1, ATP-binding cassette, sub-family B | TAP1 |
| | (MDR/TAP) | |
| 204770_at | transporter 2, ATP-binding cassette, sub-family B | TAP2 |
| | (MDR/TAP) | |
| 225973_at | transporter 2, ATP-binding cassette, sub-family B | TAP2 |
| | (MDR/TAP) | |
| 202478_at | tribbles homolog 2 (Drosophila) | TRIB2 |
| 202479_s_at | tribbles homolog 2 (Drosophila) | TRIB2 |
| 36742_at | tripartite motif-containing 15 | TRIM15 |
| 213293_s_at | tripartite motif-containing 22 | TRIM22 |
| 213884_s_at | tripartite motif-containing 3 | TRIM3 |
| 208170_s_at | tripartite motif-containing 31 | TRIM31 |
| 215444_s_at | tripartite motif-containing 31 | TRIM31 |
| 210705_s_at | tripartite motif-containing 5 | TRIM5 |
| 200628_s_at | tryptophanyl-tRNA synthetase | WARS |
| 200629_at | tryptophanyl-tRNA synthetase | WARS |
| 228882_at | tubby homolog (mouse) | TUB |
| 207490_at | tubulin, alpha 4 | TUBA4 |
| 223501_at | tumor necrosis factor (ligand) superfamily, member 13b | TNFSF13B |
| 223502_s_at | tumor necrosis factor (ligand) superfamily, member 13b | TNFSF13B |
| 1552648_a_at | tumor necrosis factor receptor superfamily, member 10a | TNFRSF10A |
| 231775_at | tumor necrosis factor receptor superfamily, member 10a | TNFRSF10A |
| 218368_s_at | tumor necrosis factor receptor superfamily, member 12A | TNFRSF12A |
| 203508_at | tumor necrosis factor receptor superfamily, member 1B | TNFRSF1B |
| 207536_s_at | tumor necrosis factor receptor superfamily, member 9 | TNFRSF9 |
| 202510_s_at | tumor necrosis factor, alpha-induced protein 2 | TNFAIP2 |
| 206026_s_at | tumor necrosis factor, alpha-induced protein 6 | TNFAIP6 |
| 220804_s_at | tumor protein p73 | TP73 |
| 232770_at | tumor suppressor candidate 3 | TUSC3 |
| 205890_s_at | ubiquitin D | UBD |
| 219211_at | ubiquitin specific protease 18 | USP18 |
| 207213_s_at | ubiquitin specific protease 2 | USP2 |
| 1562738_a_at | Ubiquitin specific protease 3 | USP3 |
| 237247_at | ubiquitin specific protease 51 | USP51 |
| 201649_at | ubiquitin-conjugating enzyme E2L 6 | UBE2L6 |
| 238657_at | UBX domain containing 3 | UBXD3 |
| 203868_s_at | vascular cell adhesion molecule 1 | VCAM1 |
| 204929_s_at | vesicle-associated membrane protein 5 (myobrevin) | VAMP5 |
| 203798_s_at | visinin-like 1 | VSNL1 |
| 1566324_a_at | v-maf musculoaponeurotic fibrosarcoma oncogene homolog | MAF |
| | (avian) | |
| 218559_s_at | v-maf musculoaponeurotic fibrosarcoma oncogene homolog | MAFB |
| | B (avian) | |
| 222670_s_at | v-maf musculoaponeurotic fibrosarcoma oncogene homolog | MAFB |
| | B (avian) | |
| 36711_at | v-maf musculoaponeurotic fibrosarcoma oncogene homolog F | MAFF |
| | (avian) | |
| 205205_at | v-rel reticuloendotheliosis viral oncogene homolog B, nuclear | RELB |
| | factor of kappa light polypeptide gene enhancer in B-cells 3 | |
| | (avian) | |
| 1557132_at | WD repeat domain 17 | WDR17 |
| 225898_at | WD repeat domain 54 | WDR54 |
| 204712_at | WNT inhibitory factor 1 | WIF1 |
| 210301_at | xanthine dehydrogenase | XDH |
| 241994_at | Xanthine dehydrogenase | XDH |
| 206133_at | XIAP associated factor-1 | BIRC4BP |
| 228617_at | XIAP associated factor-1 | BIRC4BP |
| 242234_at | XIAP associated factor-1 | BIRC4BP |
| 241588_at | YTH domain containing 2 | YTHDC2 |
| 242020_s_at | Z-DNA binding protein 1 | ZBP1 |
| 220104_at | zinc finger antiviral protein | ZAP |
| 213051_at | zinc finger antiviral protein | ZAP |
| 225634_at | zinc finger antiviral protein | ZAP |
| 218543_s_at | zinc finger CCCH type domain containing 1 | PARP12 |
| 220104_at | zinc finger CCCH type, antiviral 1 | ZC3HAV1 |
| 203603_s_at | zinc finger homeobox 1b | ZFHX1B |
| 229848_at | zinc finger protein 10 (KOX 1) | ZNF10 |
| 235366_at | zinc finger protein 10 (KOX 1) | ZNF10 |
| 229848_at | zinc finger protein 10 (KOX 1) | ZNF10 |
| 1567031_at | zinc finger protein 160 | ZNF160 |
| 220497_at | zinc finger protein 214 | ZNF214 |
| 226754_at | zinc finger protein 251 | ZNF251 |
| 1565614_at | Zinc finger protein 337 | ZNF337 |
| 201531_at | zinc finger protein 36, C3H type, homolog (mouse) | ZFP36 |
| 238454_at | zinc finger protein 540 | ZNF540 |
| 1562282_at | zinc finger protein 568 | ZNF568 |
| 155369_s_at | zinc finger protein 569 | ZNF569 |
| 228093_at | Zinc finger protein 599 | ZNF599 |
| 222816_s_at | zinc finger, CCHC domain containing 2 | ZCCHC2 |
| 1552557_a_at | zinc finger, DHHC domain containing 15 | ZDHHC15 |
| 205714_s_at | zinc finger, MYND domain containing 10 | ZMYND10 |
| 216663_s_at | zinc finger, MYND domain containing 10 | ZMYND10 |
| 1553454_at | | |
| 1556003_a_at | | |
| 1556216_s_at | | |
| 1557012_a_at | | |
| 1557236_at | | |
| 1557437_a_at | | |
| 1557617_at | | |
| 1560422_at | | |
| 1560751_at | | |
| 1561882_at | | |
| 1562472_at | | |
| 1563075_s_at | | |
| 1564656_at | | |
| 201422_at | | |
| 205442_at | | |
| 206048_at | | |
| 214084_x_at | | |
| 214511_x_at | | |
| 214712_at | | |
| 216834_at | | |
| 221159_at | | |
| 227361_at | | |
| 227783_at | | |
| 229437_at | | |
| 229543_at | | |
| 230230_at | | |
| 230776_at | | |
| 234517_at | | |
| 235276_at | | |
| 235456_at | | |
| 235539_at | | |
| 235681_at | | |
| 236915_at | | |
| 237448_at | | |
| 238491_at | | |
| 238720_at | | |
| 239302_s_at | | |
| 239896_at | | |
| 241710_at | | |
| 241857_at | | |
| 242007_at | | |
| 242620_at | | |
| 243803_at | | |
| 244045_at | | |
| 244383_at | | |

TABLE II
List of Examples for Categories of Proteins, Receptors, Enzymes, Products of
Proteins, Receptors of Products of Proteins and Expression Regulators

| Functional Categories | Examples |
|---|---|
| Antioxidative Proteins | nitric oxide synthases, ubiquitin, PARK2, catalases, |
| | protoporphyrinogen oxidase, sulfite oxidase, superoxide dismutase 2, |
| | glutathione S-transferase, superoxide dismutase 2, SOD, glutathione |
| | peroxidase |
| Antiviral | 2′,5′-oligoadenylate synthetases, viperin, phospholipid scramblase 1, |
| | adenosine deaminase, ACE2, granzymes A, B and H, GBP 1-5, |
| | interferon stimulated gene, leukemia inhibitory factor receptor, |
| | leukemia inhibitory factor, interferon-inducible double stranded RNA |
| | dependent (PRKRs) protein kinases, zinc finger antiviral proteins, zinc |
| | finger protein 10, DDX58 |
| Apoptosis | BCL-2, BCL-G, calpains, CASP1-10, Fas, Fas ligand, PMAIP1, |
| | BCL2-antagonist/killer 1, CASP8 and FADD-like apoptosis regulator, |
| | MCL1, programmed cell death ligands |
| Cell Adhesion | carbohydrate sulfotransferases, CEACAM1, catenins, c-type lectins, |
| | contactins, ficolin, integrins, ICAM1, tenascins, tetraspanins, |
| | sialoadhesins, selectins, epithelial stromal interaction 1 (EPSTI1), |
| | hyaluronan synthase 2, protocadherin 17, secreted phosphoprotein 1, |
| | lymphocyte adhesion molecule 1, TIMP1, VCAM1 |
| Cell surface molecules (Clusters | CD163, CD274, CD36, CD47, CD68, CD69, CD7, CD80, CD83, |
| of differentiation) | CD84, CD86 |
| Cellular Lysis | granulysin, granzymes A, B & H, SLAM family members (SLAMF7 |
| | &SLAMF8), syntaxins, perforins |
| Chemotaxis | CCL2, CCL3L1, CCL5, CCL8, CCL18, CCL19, XCL1, XCL2, |
| | CCL20, CXCL1, CX3CL1, CXCL2, CXCL3, CXCL5, CXCL9, |
| | CXCL10, CXCL11, CXCL13, CXCL14, MCP-1, chemokine-like |
| | factor super families 3 & 4, IP-10, |
| Chemotaxis Receptors | CCR1, CCR2, CCR3, CCR5, CCRL2, CXCR3, CXCR4, CX3CR1, |
| | XCR1 |
| Connective Tissue | Fibronectin, collagen |
| Cytokine Receptors | leukemia inhibitory factor receptor, oncostatin M receptors |
| Cytokines | Tumor necrosis factor (TNF), IL1, IL12, Type I Interferons I (IFN-a, |
| | IFN-b), IL10, IL6, IL15, IL18, Interferon-g (IFN-g)), IL19, IL-4, IL5, |
| | Transforming growth factor-b (TGF-b), Lymphotoxin (LT), IL13, |
| | CSFs, IL-28A, IL32, IL5R, IL7, IL1ra, IL-8, cystatin, defensins, |
| | SOCS1-3, TAGAP |
| Cytoskeleton and Mobility | calpains, cyclin-dependent kinase inhibitor, autotaxins, dyneins, |
| | filamins, keratins, tubulins, stomatins, tensins, tetraspanins, lamins, |
| | microtubule-associated proteins, nexillins, palmdelphins, plasminogen |
| | activators, |
| DNA replication | DNA helicase B |
| Endothelial Cell Mitogens | PD-ECGF1 |
| Extracellular Matrix | EFEMP1, hyaluronan synthase, HAPLN3, TIMP1, matrix |
| | metalloproteinases (MMPs), |
| G Protein-coupled Receptors | alpha- & beta-adrenergic receptors, succinate receptors, purinergic |
| | receptors, endothelin receptor type A, prostaglandin F receptors |
| Gap Junctions | connexins, junctophilins, claudins, cadherin |
| Immunoglobulin Receptors | Leukocyte-associated Ig-like receptors |
| Immunoglobulins | Fc Fragments of IgE and IgG |
| Inflammatory Proteins | arachidonate 5-lipoxygenase, COX, LOX, MMPs, TACE, ICE, |
| | hyaluronidase, inducible nitric oxid synthases, prostaglandins, |
| | leukotrienes |
| Interferon-induced Proteins | Interferon induced transmembrane proteins (IFITM1-IFITM3), |
| | interferon induced protein with tetratricopeptide repeats (IFIT1-IFIT5), |
| | IFI35, IFI44, IFI44L, MX1, MX2, GBP1-GBP5, IFIH-1 |
| Lipid-binding Proteins | apolipoproteins 1-6, serum amyloids, LRP2 binding protein |
| Mucin-like Hormone Receptors | EMR1, EMR2 |
| Mucins | MUC13, MUC15, sialyltransferase 4A |
| RIG-like Receptors | IFIH-1, DDX58 |
| RNA Metabolism | exoribonucleases, ribonucleases |
| Signal Transduction | JAKs, STAT1, STAT2, NFkB, phosphodiesterases, adenylate |
| | cyclases, dual specificity phosphatases, stomatin, serine threonine |
| | kinases, RIPK2, tyrosine phosphatases, Janus kinases, RGS1, RGS22, |
| | phosphodiesterases, guanylate binding proteins (GBPs), GTPases |
| TNF Receptors | tumor necrosis factor receptor superfamily |
| Toll-like Receptors | TLR2, TLR4, TLR8 |
| Transcription Factors | CREB, E1A binding protein, ETS domain transcription factor, |
| | FOSL1, EIF2AK2, interferon regulatory factors (IRF1, IRF7, IRF8), |
| | TBP-interacting protein, TIFA, Transcription factor EC, Transcription |
| | factor CP2-like |
| Transporters and Channels | CLIC2, CLIC4, sodium channels, ankyrins, calcium channel beta-3 |
| | subunit, ATP binding cassettes, ATPase, solute carrier family |
| | proteins, TAP2, TAP1 |
| Vascular Homeostasis | endothelin, endothelin receptor type A |
| Viral Receptors | ICAM1 (human RV receptor) |

What is claimed is:

1. A method for identifying compounds for regulating rhinovirus infection, comprising:
a. contacting at least one compound with a target selected from the group consisting of genes identified in Table I, proteins encoded by genes of Table I, expression regulators of genes of Table I, receptors of proteins encoded by genes of Table I, products of proteins encoded by genes of Table I, receptors of products of proteins of genes of Table I, and combinations thereof;
b. determining whether said compound binds the target; and
c. identifying those compounds that bind the target as compounds for regulating rhinovirus infection.

2. The method of claim 1, comprising at least two compounds.

3. A method for identifying compounds for regulating rhinovirus infection, comprising:
a. contacting at least one compound with a rhinovirus infection model system containing a target selected from the group consisting of genes identified in Table I, proteins encoded by genes of Table I, expression regulators of genes of Table I, receptors of proteins encoded by genes of Table I, products of proteins encoded by genes of Table I, receptors of products of proteins of genes of Table I, and combinations thereof;
b. further determining whether the compound regulates rhinovirus infection in a rhinovirus infection model system; and
c. identifying those compounds that regulate rhinovirus infection in a rhinovirus infection model system as compounds for regulating rhinovirus infection.

4. The method of claim 3, comprising at least two compounds.

5. The method of claim 1, further comprising: administering the compound identified in step (c) of claim 1 to a mammal, and determining whether the compound regulates rhinovirus infection in the mammal, wherein compounds that regulate rhinovirus infection in the mammal are identified as compounds for regulating rhinovirus infection in vivo.

6. The method of claim 1, further comprising: administering the compound identified in step (c) of claim 1 to a mammal, and determining whether the compound regulates response to rhinovirus infection in the mammal, wherein compounds that regulate response to rhinovirus infection in the mammal are identified as compounds for regulating rhinovirus infection in vivo.

7. The method of claim 3, further comprising: administering the compound identified in step (c) of claim 3 to a mammal, and determining whether the compound regulates rhinovirus infection in the mammal, wherein compounds that regulate rhinovirus infection in the mammal are identified as compounds for regulating rhinovirus infection in vivo.

8. A method for identifying compounds for regulating rhinovirus infection, comprising:
a. contacting at least one compound with a rhinovirus infection model system containing a target selected from the group consisting of genes identified in Table I, proteins encoded by genes of Table I, expression regulators of genes of Table I, receptors of proteins encoded by genes of Table I, products of proteins encoded by genes of Table I, receptors of products of proteins of genes of Table I, and combinations thereof;
b. further determining whether the compound regulates response to rhinovirus infection in a rhinovirus infection model system; and
c. identifying those compounds that regulate response to rhinovirus infection in a rhinovirus infection model system as compounds for regulating rhinovirus infection.

9. The method of claim 8, comprising at least two compounds.

10. The method of claim 8, further comprising: administering the compound identified in step (c) of claim 8 to a mammal, and determining whether the compound regulates response to rhinovirus infection in the mammal, wherein compounds that regulate response to rhinovirus infection in the mammal are identified as compounds for regulating rhinovirus infection in vivo.

11. A method for identifying compounds for regulating rhinovirus infection:
a. contacting at least one compound with a cell population expressing a protein encoded by the genes of Table I and identified in Table II
b. determining and comparing the level of activity of the protein in the cell population that is contacted with the compound to the level of activity of the protein in the cell population that is not contacted with the compound; and
c. identifying those compounds that modulate the activity of the protein in the cell population that is contacted with the compound compared to the activity in the cell population that is not contacted with the compound as compounds for regulating rhinovirus infection.

12. The method of claim 11, further comprising:
d. further determining whether the compound identified in step (c) of claim 11 regulates rhinovirus infection in a rhinovirus infection model system; and
e. identifying those compounds that regulate rhinovirus infection in a rhinovirus infection model system as compounds for regulating rhinovirus infection.

13. The method of claim 11, further comprising: administering the compound identified in step (c) of claim 11 to a mammal, and determining whether the compound regulates rhinovirus infection in the mammal, wherein compounds that regulate rhinovirus infection in the mammal are identified as compounds for regulating rhinovirus infection.

14. The method of claim 12, further comprising: administering the compound identified in step (e) of claim 12 to a mammal, and determining whether the compound regulates rhinovirus infection in the mammal, wherein compounds that regulate rhinovirus infection in the mammal are identified as compounds for regulating rhinovirus infection.

15. A method for identifying compounds for regulating a rhinovirus infection, comprising:
a. contacting at least one compound with a cell population expressing a protein encoded by genes of Table I and identified in Table II;
b. determining and comparing the level of expression of the protein in the cell population that is contacted with the compound to the level of expression of the protein in the cell population that is not contacted with the compound; and
c. identifying those compounds that modulate the expression of the protein in the cell population that is contacted with the compound compared to the expression of the protein in the cell population that is not contacted with the compound as compounds for regulating rhinovirus infection.

16. The method of claim 15, further comprising:
d. determining whether the compound identified in step (c) of claim 15 regulates rhinovirus infection in a rhinovirus infection model system; and
e. identifying those compounds that regulate rhinovirus infection in a rhinovirus infection model system as compounds for regulating rhinovirus infection.

17. The method of claim 15, further comprising: administering the compound identified in step (c) of claim 15 to a mammal, and determining whether the compound regulates rhinovirus infection in the mammal, wherein compounds that regulate rhinovirus infection in the mammal are identified as compounds for regulating rhinovirus infection.

18. The method of claim 16, further comprising: administering the compound identified in step (e) of claim 16 to a mammal, and determining whether the compound regulates rhinovirus infection in the mammal, wherein compounds that regulate rhinovirus infection in the mammal are identified as compounds for regulating rhinovirus infection.

19. A method for identifying compounds for regulating rhinovirus infection, comprising:
a. contacting at least one compound with a cell population expressing a gene identified in Table I;
b. determining and comparing the level of expression of the gene in the cell population that is contacted with the compound to the level of expression of the gene in the cell population that is not contacted with the compound; and
c. identifying those compounds that modulate the expression of the gene in the cell population that is contacted with the compound compared to the expression of the gene in the cell population that is not contacted with the compound as compounds for regulating rhinovirus infection.

20. The method of claim 19, further comprising:
d. determining whether the compound identified in step (c) of claim 19 regulates rhinovirus infection in a rhinovirus infection model system; and
e. identifying those compounds that regulate rhinovirus infection in a rhinovirus infection model system as compounds for regulating rhinovirus infection.

21. The method of claim 19, further comprising: administering the compound identified in step (c) of claim 19 to a mammal, and determining whether the compound regulates rhinovirus infection in the mammal, wherein compounds that regulate rhinovirus infection in the mammal are identified as compounds for regulating rhinovirus infection.

22. The method of claim 20, further comprising: administering the compound identified in step (e) of claim 20 to a mammal, and determining whether the compound regulates rhinovirus infection in the mammal, wherein compounds that regulate rhinovirus infection in the mammal are identified as compounds for regulating rhinovirus infection.

23. A method of diagnosing a rhinovirus infection, comprising:
a. determining in a biological sample an expression profile for one or more targets selected from the group involved in rhinovirus infection identified in Tables I and Table II in a biological sample; or measuring the level of expression or activity of one or more proteins involved in regulating rhinovirus infection identified in Table II in a biological sample;
b. comparing levels of expression of one or more targets identified in a biological sample to levels of expression of one or more targets from a control sample or database, or comparing levels of expression or activity profile of the proteins from the sample to levels of expression or activity profile of the proteins from a control sample or from a database, wherein significant deviation from control levels is indicative of symptom development in rhinovirus infection.

24. A method of monitoring progression of rhinovirus infection, comprising:
a. determining a gene expression profile for one or more gene involved in regulating rhinovirus infection identified in Table I in a biological sample; or preparing a protein expression profile, or protein activity profile of one or more proteins involved in regulating rhinovirus infection identified in Table I in a biological sample from a suitable rhinovirus infection model system;
b. preparing a similar expression or activity profile as in step (a) after a suitable time after the therapeutic regimen;
c. repeating step (b) during the course of the therapy and evaluating the data to monitor progression of rhinovirus infection.

25. A method of monitoring the treatment or progression of a disorder in a patient with symptom development in rhinovirus infection, comprising:
a. determining a gene expression profile for one or more genes involved in regulating rhinovirus infection identified in Table I in a biological sample; or preparing a protein expression profile, or protein activity profile of one or more proteins involved in regulating rhinovirus infection identified in Table I in a biological sample from a subject;
b. administering a therapeutic regimen to the subject;
c. preparing a similar expression or activity profile as in step (a) from a biological sample from the subject after a suitable time after the therapeutic regimen;
d. comparing the profiles prior to the therapy with profiles after the therapy; and
e. repeating the steps (b), (c) and (d) during the course of the treatment or disorder and evaluating the data to monitor efficacy of the treatment or progression of the disorder.

26. A medicinal composition, comprising:
a. a safe and effective amount of at least one compound identified by the method of claim 1; and
b. a pharmaceutically acceptable carrier.

27. A medicinal composition comprising a safe and effective amount of at least one compound identified by the method of claim 1.

28. A medicinal composition comprising a safe and effective amount of at least one compound identified by the method of claim 3.

29. A medicinal composition comprising a safe and effective amount of at least one compound identified by the method of claim 8.

30. A medicinal composition comprising a safe and effective amount of at least one compound identified by the method of claim 11.

31. A medicinal composition comprising a safe and effective amount of at least one compound identified by the method of claim 15.

32. A medicinal composition comprising a safe and effective amount of at least one compound identified by the method of claim 19.

33. A method for regulating rhinovirus infection in a subject in which such regulation is desirable, comprising:
a. identifying a subject in which regulation of rhinovirus infection is desirable; and
b. administering to the subject a safe and effective amount of at least one compound identified by the method of claim 1.

34. The method of claim 33, wherein the desired regulation of rhinovirus infection is a decrease in rhinovirus infection in the subject.

35. A method for regulating rhinovirus infection in a subject in which such regulation is desirable, comprising:
a. identifying a subject in which regulation of rhinovirus infection is desirable; and
b. administering to the subject a safe and effective amount of at least one compound identified by the method of claim 3.

36. The method of claim 35, wherein the desired regulation of rhinovirus infection is a decrease in rhinovirus infection in the subject.
